SEQUENCE LISTING (36 sequences)

SEQ ID NO: 1 - protein, 1532 aa, Homo sapiens
MGHSKQIRILLLNEMEKLEKTLFRLEQGYELQFRLGPTLQGKAVTVYTNYPFPGETFNREKFRSLDWENPTEREDDSDKYCKLNLQQSGSFQYYFLQGNEKSGGGYIVVDPILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVSRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLASRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPDDMVYCGIYDNALDNDNYNLAKGFNYHQGPEWLWPIGYFLRAKLYFSRLMGPETTAKTIVLVKNVLSRHYVHLERSPWKGLPELTNENAQYCPFSCETQAWSIATILETLYDL

SEQ ID NO: 2 - protein, 1515 aa, Homo sapiens
MSLLTCAFYLGYELQFRLGPTLQGKAVTVYTNYPFPGETFNREKFRSLDWENPTEREDDSDKYCKLNLQQSGSFQYYFLQGNEKSGGGYIVVDPILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVSRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLASRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPDDMVYCGIYDNALDNDNYNLAKGFNYHQGPEWLWPIGYFLRAKLYFSRLMGPETTAKTIVLVKNVLSRHYVHLERSPWKGLPELTNENAQYCPFSCETQAWSIATILETLYDL

SEQ ID NO: 3 - protein, 1516 aa, Homo sapiens
MAPILSINLFIGYELQFRLGPTLQGKAVTVYTNYPFPGETFNREKFRSLDWENPTEREDDSDKYCKLNLQQSGSFQYYFLQGNEKSGGGYIVVDPILRVGADNHVLPLDCVTLQTFLAKCLGPFDEWESRLRVAKESGYNMIHFTPLQTLGLSRSCYSLANQLELNPDFSRPNRKYTWNDVGQLVEKLKKEWNVICITDVVYNHTAANSKWIQEHPECAYNLVNSPHLKPAWVLDRALWRFSCDVAEGKYKEKGIPALIENDHHMNSIRKIIWEDIFPKLKLWEFFQVDVNKAVEQFRRLLTQENRRVTKSDPNQHLTIIQDPEYRRFGCTVDMNIALTTFIPHDKGPAAIEECCNWFHKRMEELNSEKHRLINYHQEQAVNCLLGNVFYERLAGHGPKLGPVTRKHPLVTRYFTFPFEEIDFSMEESMIHLPNKACFLMAHNGWVMGDDPLRNFAEPGSEVYLRRELICWGDSVKLRYGNKPEDCPYLWAHMKKYTEITATYFQGVRLDNCHSTPLHVAEYMLDAARNLQPNLYVVAELFTGSEDLDNVFVTRLGISSLIREAMSAYNSHEEGRLVYRYGGEPVGSFVQPCLRPLMPAIAHALFMDITHDNECPIVHRSAYDALPSTTIVSMACCASGSTRGYDELVPHQISVVSEERFYTKWNPEALPSNTGEVNFQSGIIAARCAISKLHQELGAKGFIQVYVDQVDEDIVAVTRHSPSIHQSVVAVSRTAFRNPKTSFYSKEVPQMCIPGKIEEVVLEARTIERNTKPYRKDENSINGTPDITVEIREHIQLNESKIVKQAGVATKGPNEYIQEIEFENLSPGSVIIFRVSLDPHAQVAVGILRNHLTQFSPHFKSGSLAVDNADPILKIPFASLASRLTLAELNQILYRCESEEKEDGGGCYDIPNWSALKYAGLQGLMSVLAEIRPKNDLGHPFCNNLRSGDWMIDYVSNRLISRSGTIAEVGKWLQAMFFYLKQIPRYLIPCYFDAILIGAYTTLLDTAWKQMSSFVQNGSTFVKHLSLGSVQLCGVGKFPSLPILSPALMDVPYRLNEITKEKEQCCVSLAAGLPHFSSGIFRCWGRDTFIALRGILLITGRYVEARNIILAFAGTLRHGLIPNLLGEGIYARYNCRDAVWWWLQCIQDYCKMVPNGLDILKCPVSRMYPTDDSAPLPAGTLDQPLFEVIQEAMQKHMQGIQFRERNAGPQIDRNMKDEGFNITAGVDEETGFVYGGNRFNCGTWMDKMGESDRARNRGIPATPRDGSAVEIVGLSKSAVRWLLELSKKNIFPYHEVTVKRHGKAIKVSYDEWNRKIQDNFEKLFHVSEDPSDLNEKHPNLVHKRGIYKDSYGASSPWCDYQLRPNFTIAMVVAPELFTTEKAWKALEIAEKKLLGPLGMKTLDPDDMVYCGIYDNALDNDNYNLAKGFNYHQGPEWLWPIGYFLRAKLYFSRLMGPETTAKTIVLVKNVLSRHYVHLERSPWKGLPELTNENAQYCPFSCETQAWSIATILETLYDL

SEQ ID NO: 4 - protein, 952 aa, Homo sapiens [sequence omitted]

SEQ ID NO: 5 - protein, 957 aa, Homo sapiens [sequence omitted]

SEQ ID NO: 6 - protein, 116 aa, Artificial Sequence
  Description of Artificial Sequence Synthetic polypeptide
EVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSS

SEQ ID NO: 7 - protein, 15 aa, Artificial Sequence
  Description of Artificial Sequence Synthetic peptide
GGGGSGGGGSGGGGS

SEQ ID NO: 8 - protein, 111 aa, Artificial Sequence
  Description of Artificial Sequence Synthetic polypeptide
DIVLTQSPASLAVSLGQRATISCRASKSVSTSSYSYMHWYQQKPGQPPKLLIKYASYLESGVPARFSGSGSGTDFHLNIHPVEEEDAATYYCQHSREFPWTFGGGTKLELK

SEQ ID NO: 9 - protein, 5 aa, Artificial Sequence
  Description of Artificial Sequence Synthetic peptide
NYGMH

SEQ ID NO: 10 - protein, 17 aa, Artificial Sequence
  Description of Artificial Sequence Synthetic peptide
YISSGSSTIYYADTVKG

SEQ ID NO: 11 - protein, 7 aa, Artificial Sequence
  Description of Artificial Sequence Synthetic peptide
RGLLLDY

SEQ ID NO: 12 - protein, 15 aa, Artificial Sequence
  Description of Artificial Sequence Synthetic peptide
RASKSVSTSSYSYMH

SEQ ID NO: 13 - protein, 7 aa, Artificial Sequence
  Description of Artificial Sequence Synthetic peptide
YASYLES

SEQ ID NO: 14 - protein, 9 aa, Artificial Sequence
  Description of Artificial Sequence Synthetic peptide
QHSREFPWT

SEQ ID NO: 15 - protein, 660 aa, Artificial Sequence
  Description of Artificial Sequence Synthetic polypeptide
GQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEA

SEQ ID NO: 16 - protein, 495 aa, Artificial Sequence
  Description of Artificial Sequence Synthetic polypeptide
GANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEA

SEQ ID NO: 17 - DNA, 7371 bp, Homo sapiens
cccggaagtgggccagaggtacggtccgctcccacctggggcgagtgcgcgcacggccaggttgggtaccgggtgcgcccaggaacccgcgcgaggcgaagtcgctgagactctgcctgcttctcacccagctgcctcggcgctgccccggtcgctcgccgcccctccctttgcccttcacggcgcccggccctccttgggctgcggcttctgtgcgaggctgggcagccagcccttccccttctgtttctccccgtcccctccccccgaccgtagcaccagagtcgcgggtcctgcagtgccccagaagccgcacgtataactccctcggcgggtaactcattcgactgtggagttcttttaattcttatgaaagatttcaaatcctctagaagccaaaatgggacacagtaaacagattcgaattttacttctgaacgaaatggagaaactggaaaagaccctcttcagacttgaacaagggtatgagctacagttccgattaggcccaactttacagggaaaagcagttaccgtgtatacaaattacccatttcctggagaaacatttaatagagaaaaattccgttctctggattgggaaaatccaacagaaagagaagatgattctgataaatactgtaaacttaatctgcaacaatctggttcatttcagtattatttccttcaaggaaatgagaaaagtggtggaggttacatagttgtggaccccattttacgtgttggtgctgataatcatgtgctacccttggactgtgttactcttcagacatttttagctaagtgtttgggaccttttgatgaatgggaaagcagacttagggttgcaaaagaatcaggctacaacatgattcattttaccccattgcagactcttggactatctaggtcatgctactcccttgccaatcagttagaattaaatcctgacttttcaagacctaatagaaagtatacctggaatgatgttggacagctagtggaaaaattaaaaaaggaatggaatgttatttgtattactgatgttgtctacaatcatactgctgctaatagtaaatggatccaggaacatccagaatgtgcctataatcttgtgaattctccacacttaaaacctgcctgggtcttagacagagcactttggcgtttctcctgtgatgttgcagaagggaaatacaaagaaaagggaatacctgctttgattgaaaatgatcaccatatgaattccatccgaaaaataatttgggaggatatttttccaaagcttaaactctgggaatttttccaagtagatgtcaacaaagcggttgagcaatttagaagacttcttacacaagaaaataggcgagtaaccaagtctgatccaaaccaacaccttacgattattcaagatcctgaatacagacggtttggctgtactgtagatatgaacattgcactaacgactttcataccacatgacaaggggccagcagcaattgaagaatgctgtaattggtttcataaaagaatggaggaattaaattcagagaagcatcgactcattaactatcatcaggaacaggcagttaattgccttttgggaaatgtgttttatgaacgactggctggccatggtccaaaactaggacctgtcactagaaagcatcctttagttaccaggtattttactttcccatttgaagagatagacttctccatggaagaatctatgattcatctgccaaataaagcttgttttctgatggcacacaatggatgggtaatgggagatgatcctcttcgaaactttgctgaaccgggttcagaagtttacctaaggagagaacttatttgctggggagacagtgttaaattacgctatgggaataaaccagaggactgtccttatctctgggcacacatgaaaaaatacactgaaataactgcaacttatttccagggagtacgtcttgataactgccactcaacacctcttcacgtagctgagtacatgttggatgctgctaggaatttgcaacccaatttatatgtagtagctgaactgttcacaggaagtgaagatctggacaatgtctttgttactagactgggcattagttccttaataagagaggcaatgagtgcatataatagtcatgaagagggcagattagtttaccgatatggaggagaacctgttggatcctttgttcagccctgtttgaggcctttaatgccagctattgcacatgccctgtttatggatattacgcatgataatgagtgtcctattgtgcatagatcagcgtatgatgctcttccaagtactacaattgtttctatggcatgttgtgctagtggaagtacaagaggctatgatgaattagtgcctcatcagatttcagtggtttctgaagaacggttttacactaagtggaatcctgaagcattgccttcaaacacaggtgaagttaatttccaaagcggcattattgcagccaggtgtgctatcagtaaacttcatcaggagcttggagccaagggttttattcaggtgtatgtggatcaagttgatgaagacatagtggcagtaacaagacactcacctagcatccatcagtctgttgtggctgtatctagaactgctttcaggaatcccaagacttcattttacagcaaggaagtgcctcaaatgtgcatccctggcaaaattgaagaagtagttcttgaagctagaactattgagagaaacacgaaaccttataggaaggatgagaattcaatcaatggaacaccagatatcacagtagaaattagagaacatattcagcttaatgaaagtaaaattgttaaacaagctggagttgccacaaaagggcccaatgaatatattcaagaaatagaatttgaaaacttgtctccaggaagtgttattatattcagagttagtcttgatccacatgcacaagtcgctgttggaattcttcgaaatcatctgacacaattcagtcctcactttaaatctggcagcctagctgttgacaatgcagatcctatattaaaaattccttttgcttctcttgcctccagattaactttggctgagctaaatcagatcctttaccgatgtgaatcagaagaaaaggaagatggtggagggtgctatgacataccaaactggtcagcccttaaatatgcaggtcttcaaggtttaatgtctgtattggcagaaataagaccaaagaatgacttggggcatcctttttgtaataatttgagatctggagattggatgattgactatgtcagtaaccggcttatttcacgatcaggaactattgctgaagttggtaaatggttgcaggctatgttcttctacctgaagcagatcccacgttaccttatcccatgttactttgatgctatattaattggtgcatataccactcttctggatacagcatggaagcagatgtcaagctttgttcagaatggttcaacctttgtgaaacacctttcattgggttcagttcaactgtgtggagtaggaaaattcccttccctgccaattctttcacctgccctaatggatgtaccttataggttaaatgagatcacaaaagaaaaggagcaatgttgtgtttctctagctgcaggcttacctcatttttcttctggtattttccgctgctggggaagggatacttttattgcacttagaggtatactgctgattactggacgctatgtagaagccaggaatattattttagcatttgcgggtaccctgaggcatggtctcattcctaatctactgggtgaaggaatttatgccagatacaattgtcgggatgctgtgtggtggtggctgcagtgtatccaggattactgtaaaatggttccaaatggtctagacattctcaagtgcccagtttccagaatgtatcctacagatgattctgctcctttgcctgctggcacactggatcagccattgtttgaagtcatacaggaagcaatgcaaaaacacatgcagggcatacagttccgagaaaggaatgctggtccccagatagatcgaaacatgaaggacgaaggttttaatataactgcaggagttgatgaagaaacaggatttgtttatggaggaaatcgtttcaattgtggcacatggatggataaaatgggagaaagtgacagagctagaaacagaggaatcccagccacaccaagagatgggtctgctgtggaaattgtgggcctgagtaaatctgctgttcgctggttgctggaattatccaaaaaaaatattttcccttatcatgaagtcacagtaaaaagacatggaaaggctataaaggtctcatatgatgagtggaacagaaaaatacaagacaactttgaaaagctatttcatgtttccgaagacccttcagatttaaatgaaaagcatccaaatctggttcacaaacgtggcatatacaaagatagttatggagcttcaagtccttggtgtgactatcagctcaggcctaattttaccatagcaatggttgtggcccctgagctctttactacagaaaaagcatggaaagctttggagattgcagaaaaaaaattgcttggtccccttggcatgaaaactttagatccagatgatatggtttactgtggaatttatgacaatgcattagacaatgacaactacaatcttgctaaaggtttcaattatcaccaaggacctgagtggctgtggcctattgggtattttcttcgtgcaaaattatatttttccagattgatgggcccggagactactgcaaagactatagttttggttaaaaatgttctttcccgacattatgttcatcttgagagatccccttggaaaggacttccagaactgaccaatgagaatgcccagtactgtcctttcagctgtgaaacacaagcctggtcaattgctactattcttgagacactttatgatttatagtttattacagatattaagtatgcaattacttgtattataggatgcaaggtcatcatatgtaaatgccttatatgcacaggctcaagttgttttaaaaatctcatttattataatattgatgctcaattaggtaagattgtaaaagcattgattttttttaatgtacagaggtagatttcaatttgaatcagaaagaaatatcattaccaatgaaatgtgtttgagttcagtaagaattattcaaatgcctagaaatccatagtttggaaaagaaaaatcatgtcatcttctatttgtacagaaatgaaaataaaatatgaaaataatgaaagaaatgaaaagatagcttttaattgtggtatatataatcttcagtaacaatacatactgaatacgctgtggttcattaatattaacaccacgtactatagtattcttaatacagtgctcactgcatttaataaatatttaataaatgatgaatgatagaagtttccatctacaatatatgttcctaaatggagcacagatgttcaaactatgctttcattttttcactgatatattaatttttgtgtaatgaatgccaacagtatattttatatgatttacttatgtgaggaaacatgcaaagcattaggaaatttatttcctaaaaacagttttgtaaaattagtattgagttctattgagtattataagatagcttacattttcaaaatggaaattgtcggtcatatttctagaactttaaagaaaaaagaatgttatattagttttctaaaactcaactatctttagtcatgttcaaaaatctattgctagatcatagtagatactggttttctattaactcaaaacctacattgacaagtttaacattgagaagaatcttaacaaaaatatggatatgaattcagtagatatcttaaattcaataaaatcactggaagtttttcatgataacttattttaagatgccttaaaaatcttaaagtcacaaaaggaaaaaggtttttaacatttacatgagttaacattttttcatagaacttatttcctagatagaattttttactgttttttactgttttcttaagaaaacagttaaatcattatgcattcagttggaagaaagtagtggcaagaattctttcattgctatataatattcagtggctcatttatacctaataaaataatggtattttaaaataatgctactttcaaagtagcatttttttagttagtttacaggttacatacccaaaaccttaactatgactaagaaattaaagaagaaaaccagcaaactaaaacttctgggcagcaaaaatatataaatgcttcagatgtcaaatacccatgcttgaaagctcgtgtaatttactttaagattatctgcctgctcttcttcaaagctgaccttgctttagaaatagttttaactagcttagttttctggtttccaaaactaaaatagattaaatcctacaaatttaaggacagttgtgacagtaatctgaccactatctataaatacattggacattggtttccaaatctccctttcttcttcagttccttccttgttcaatatatacccttctctaaactgtgcgggtaaaaggaatgactgtccttgagagaaccattagtttatcaaaggtttatgtagttttgttgctgtaccctaactttgatattcagggaggtaggaaaggtaacagaaaaccagcatatttaatcaaagcaagaagtaatcgctgacagttaaatgtgaccaaaaaaattaaaagttcacaatttttttaatgtagccatttggggttatctctagtaaggcagatacccacgttggtaaatttttaggatattgtgttgcactagaaaactaagtggttcatatttctaatgaggaagattaatgaaagaacattgttatattctgcgtggtatattttaaagtttaagaaggcatgttaaacattatttcctctatggtagttaaaatacagaattagatttttaacaggtgtcatttgactaaacgtttcggtagaatgcttcatacttgagtgatgctggataaggtattgtatttcaacaatggactatgccttggtttttcactaatcaaaatcaaaattactctttaacatgataaatgaatttaccagtttagtatgctgtggtattttaataagttttcaaagataattgggaaaacatgagactggtcatattgatgaatattgtaacatgtgaattgtgatccatttctgatatgtcttgaactactgtgtctagtgggcaaatgtcattgttacctctgtgtgttaagaaaataaaaatattttctaaaggtctgt

SEQ ID NO: 18 - DNA, 7109 bp, Homo sapiens
ctgtctacggcagctattccagaggcaacaactgcttccctctgttctcatctccccattggtggctggcgacccgaatttgggaatggggagattgcccacctgttatctttgagcagactaatctcttaagccaaaatgggacacagtaaacagattcgaattttacttctgaacgaaatggagaaactggaaaagaccctcttcagacttgaacaagggtatgagctacagttccgattaggcccaactttacagggaaaagcagttaccgtgtatacaaattacccatttcctggagaaacatttaatagagaaaaattccgttctctggattgggaaaatccaacagaaagagaagatgattctgataaatactgtaaacttaatctgcaacaatctggttcatttcagtattatttccttcaaggaaatgagaaaagtggtggaggttacatagttgtggaccccattttacgtgttggtgctgataatcatgtgctacccttggactgtgttactcttcagacatttttagctaagtgtttgggaccttttgatgaatgggaaagcagacttagggttgcaaaagaatcaggctacaacatgattcattttaccccattgcagactcttggactatctaggtcatgctactcccttgccaatcagttagaattaaatcctgacttttcaagacctaatagaaagtatacctggaatgatgttggacagctagtggaaaaattaaaaaaggaatggaatgttatttgtattactgatgttgtctacaatcatactgctgctaatagtaaatggatccaggaacatccagaatgtgcctataatcttgtgaattctccacacttaaaacctgcctgggtcttagacagagcactttggcgtttctcctgtgatgttgcagaagggaaatacaaagaaaagggaatacctgctttgattgaaaatgatcaccatatgaattccatccgaaaaataatttgggaggatatttttccaaagcttaaactctgggaatttttccaagtagatgtcaacaaagcggttgagcaatttagaagacttcttacacaagaaaataggcgagtaaccaagtctgatccaaaccaacaccttacgattattcaagatcctgaatacagacggtttggctgtactgtagatatgaacattgcactaacgactttcataccacatgacaaggggccagcagcaattgaagaatgctgtaattggtttcataaaagaatggaggaattaaattcagagaagcatcgactcattaactatcatcaggaacaggcagttaattgccttttgggaaatgtgttttatgaacgactggctggccatggtccaaaactaggacctgtcactagaaagcatcctttagttaccaggtattttactttcccatttgaagagatagacttctccatggaagaatctatgattcatctgccaaataaagcttgttttctgatggcacacaatggatgggtaatgggagatgatcctcttcgaaactttgctgaaccgggttcagaagtttacctaaggagagaacttatttgctggggagacagtgttaaattacgctatgggaataaaccagaggactgtccttatctctgggcacacatgaaaaaatacactgaaataactgcaacttatttccagggagtacgtcttgataactgccactcaacacctcttcacgtagctgagtacatgttggatgctgctaggaatttgcaacccaatttatatgtagtagctgaactgttcacaggaagtgaagatctggacaatgtctttgttactagactgggcattagttccttaataagagaggcaatgagtgcatataatagtcatgaagagggcagattagtttaccgatatggaggagaacctgttggatcctttgttcagccctgtttgaggcctttaatgccagctattgcacatgccctgtttatggatattacgcatgataatgagtgtcctattgtgcatagatcagcgtatgatgctcttccaagtactacaattgtttctatggcatgttgtgctagtggaagtacaagaggctatgatgaattagtgcctcatcagatttcagtggtttctgaagaacggttttacactaagtggaatcctgaagcattgccttcaaacacaggtgaagttaatttccaaagcggcattattgcagccaggtgtgctatcagtaaacttcatcaggagcttggagccaagggttttattcaggtgtatgtggatcaagttgatgaagacatagtggcagtaacaagacactcacctagcatccatcagtctgttgtggctgtatctagaactgctttcaggaatcccaagacttcattttacagcaaggaagtgcctcaaatgtgcatccctggcaaaattgaagaagtagttcttgaagctagaactattgagagaaacacgaaaccttataggaaggatgagaattcaatcaatggaacaccagatatcacagtagaaattagagaacatattcagcttaatgaaagtaaaattgttaaacaagctggagttgccacaaaagggcccaatgaatatattcaagaaatagaatttgaaaacttgtctccaggaagtgttattatattcagagttagtcttgatccacatgcacaagtcgctgttggaattcttcgaaatcatctgacacaattcagtcctcactttaaatctggcagcctagctgttgacaatgcagatcctatattaaaaattccttttgcttctcttgcctccagattaactttggctgagctaaatcagatcctttaccgatgtgaatcagaagaaaaggaagatggtggagggtgctatgacataccaaactggtcagcccttaaatatgcaggtcttcaaggtttaatgtctgtattggcagaaataagaccaaagaatgacttggggcatcctttttgtaataatttgagatctggagattggatgattgactatgtcagtaaccggcttatttcacgatcaggaactattgctgaagttggtaaatggttgcaggctatgttcttctacctgaagcagatcccacgttaccttatcccatgttactttgatgctatattaattggtgcatataccactcttctggatacagcatggaagcagatgtcaagctttgttcagaatggttcaacctttgtgaaacacctttcattgggttcagttcaactgtgtggagtaggaaaattcccttccctgccaattctttcacctgccctaatggatgtaccttataggttaaatgagatcacaaaagaaaaggagcaatgttgtgtttctctagctgcaggcttacctcatttttcttctggtattttccgctgctggggaagggatacttttattgcacttagaggtatactgctgattactggacgctatgtagaagccaggaatattattttagcatttgcgggtaccctgaggcatggtctcattcctaatctactgggtgaaggaatttatgccagatacaattgtcgggatgctgtgtggtggtggctgcagtgtatccaggattactgtaaaatggttccaaatggtctagacattctcaagtgcccagtttccagaatgtatcctacagatgattctgctcctttgcctgctggcacactggatcagccattgtttgaagtcatacaggaagcaatgcaaaaacacatgcagggcatacagttccgagaaaggaatgctggtccccagatagatcgaaacatgaaggacgaaggttttaatataactgcaggagttgatgaagaaacaggatttgtttatggaggaaatcgtttcaattgtggcacatggatggataaaatgggagaaagtgacagagctagaaacagaggaatcccagccacaccaagagatgggtctgctgtggaaattgtgggcctgagtaaatctgctgttcgctggttgctggaattatccaaaaaaaatattttcccttatcatgaagtcacagtaaaaagacatggaaaggctataaaggtctcatatgatgagtggaacagaaaaatacaagacaactttgaaaagctatttcatgtttccgaagacccttcagatttaaatgaaaagcatccaaatctggttcacaaacgtggcatatacaaagatagttatggagcttcaagtccttggtgtgactatcagctcaggcctaattttaccatagcaatggttgtggcccctgagctctttactacagaaaaagcatggaaagctttggagattgcagaaaaaaaattgcttggtccccttggcatgaaaactttagatccagatgatatggtttactgtggaatttatgacaatgcattagacaatgacaactacaatcttgctaaaggtttcaattatcaccaaggacctgagtggctgtggcctattgggtattttcttcgtgcaaaattatatttttccagattgatgggcccggagactactgcaaagactatagttttggttaaaaatgttctttcccgacattatgttcatcttgagagatccccttggaaaggacttccagaactgaccaatgagaatgcccagtactgtcctttcagctgtgaaacacaagcctggtcaattgctactattcttgagacactttatgatttatagtttattacagatattaagtatgcaattacttgtattataggatgcaaggtcatcatatgtaaatgccttatatgcacaggctcaagttgttttaaaaatctcatttattataatattgatgctcaattaggtaagattgtaaaagcattgattttttttaatgtacagaggtagatttcaatttgaatcagaaagaaatatcattaccaatgaaatgtgtttgagttcagtaagaattattcaaatgcctagaaatccatagtttggaaaagaaaaatcatgtcatcttctatttgtacagaaatgaaaataaaatatgaaaataatgaaagaaatgaaaagatagcttttaattgtggtatatataatcttcagtaacaatacatactgaatacgctgtggttcattaatattaacaccacgtactatagtattcttaatacagtgctcactgcatttaataaatatttaataaatgatgaatgatagaagtttccatctacaatatatgttcctaaatggagcacagatgttcaaactatgctttcattttttcactgatatattaatttttgtgtaatgaatgccaacagtatattttatatgatttacttatgtgaggaaacatgcaaagcattaggaaatttatttcctaaaaacagttttgtaaaattagtattgagttctattgagtattataagatagcttacattttcaaaatggaaattgtcggtcatatttctagaactttaaagaaaaaagaatgttatattagttttctaaaactcaactatctttagtcatgttcaaaaatctattgctagatcatagtagatactggttttctattaactcaaaacctacattgacaagtttaacattgagaagaatcttaacaaaaatatggatatgaattcagtagatatcttaaattcaataaaatcactggaagtttttcatgataacttattttaagatgccttaaaaatcttaaagtcacaaaaggaaaaaggtttttaacatttacatgagttaacattttttcatagaacttatttcctagatagaattttttactgttttttactgttttcttaagaaaacagttaaatcattatgcattcagttggaagaaagtagtggcaagaattctttcattgctatataatattcagtggctcatttatacctaataaaataatggtattttaaaataatgctactttcaaagtagcatttttttagttagtttacaggttacatacccaaaaccttaactatgactaagaaattaaagaagaaaaccagcaaactaaaacttctgggcagcaaaaatatataaatgcttcagatgtcaaatacccatgcttgaaagctcgtgtaatttactttaagattatctgcctgctcttcttcaaagctgaccttgctttagaaatagttttaactagcttagttttctggtttccaaaactaaaatagattaaatcctacaaatttaaggacagttgtgacagtaatctgaccactatctataaatacattggacattggtttccaaatctccctttcttcttcagttccttccttgttcaatatatacccttctctaaactgtgcgggtaaaaggaatgactgtccttgagagaaccattagtttatcaaaggtttatgtagttttgttgctgtaccctaactttgatattcagggaggtaggaaaggtaacagaaaaccagcatatttaatcaaagcaagaagtaatcgctgacagttaaatgtgaccaaaaaaattaaaagttcacaatttttttaatgtagccatttggggttatctctagtaaggcagatacccacgttggtaaatttttaggatattgtgttgcactagaaaactaagtggttcatatttctaatgaggaagattaatgaaagaacattgttatattctgcgtggtatattttaaagtttaagaaggcatgttaaacattatttcctctatggtagttaaaatacagaattagatttttaacaggtgtcatttgactaaacgtttcggtagaatgcttcatacttgagtgatgctggataaggtattgtatttcaacaatggactatgccttggtttttcactaatcaaaatcaaaattactctttaacatgataaatgaatttaccagtttagtatgctgtggtattttaataagttttcaaagataattgggaaaacatgagactggtcatattgatgaatattgtaacatgtgaattgtgatccatttctgatatgtcttgaactactgtgtctagtgggcaaatgtcattgttacctctgtgtgttaagaaaataaaaatattttctaaaggtctgt

SEQ ID NO: 19 - DNA, 7169 bp, Homo sapiens
ctgtctacggcagctattccagaggcaacaactgcttccctctgttctcatctccccattggtggctggcgacccgaatttgggaatggggagattgcccacctgttatctttgagcagactaatctcttgggtaactcattcgactgtggagttcttttaattcttatgaaagatttcaaatcctctagaagccaaaatgggacacagtaaacagattcgaattttacttctgaacgaaatggagaaactggaaaagaccctcttcagacttgaacaagggtatgagctacagttccgattaggcccaactttacagggaaaagcagttaccgtgtatacaaattacccatttcctggagaaacatttaatagagaaaaattccgttctctggattgggaaaatccaacagaaagagaagatgattctgataaatactgtaaacttaatctgcaacaatctggttcatttcagtattatttccttcaaggaaatgagaaaagtggtggaggttacatagttgtggaccccattttacgtgttggtgctgataatcatgtgctacccttggactgtgttactcttcagacatttttagctaagtgtttgggaccttttgatgaatgggaaagcagacttagggttgcaaaagaatcaggctacaacatgattcattttaccccattgcagactcttggactatctaggtcatgctactcccttgccaatcagttagaattaaatcctgacttttcaagacctaatagaaagtatacctggaatgatgttggacagctagtggaaaaattaaaaaaggaatggaatgttatttgtattactgatgttgtctacaatcatactgctgctaatagtaaatggatccaggaacatccagaatgtgcctataatcttgtgaattctccacacttaaaacctgcctgggtcttagacagagcactttggcgtttctcctgtgatgttgcagaagggaaatacaaagaaaagggaatacctgctttgattgaaaatgatcaccatatgaattccatccgaaaaataatttgggaggatatttttccaaagcttaaactctgggaatttttccaagtagatgtcaacaaagcggttgagcaatttagaagacttcttacacaagaaaataggcgagtaaccaagtctgatccaaaccaacaccttacgattattcaagatcctgaatacagacggtttggctgtactgtagatatgaacattgcactaacgactttcataccacatgacaaggggccagcagcaattgaagaatgctgtaattggtttcataaaagaatggaggaattaaattcagagaagcatcgactcattaactatcatcaggaacaggcagttaattgccttttgggaaatgtgttttatgaacgactggctggccatggtccaaaactaggacctgtcactagaaagcatcctttagttaccaggtattttactttcccatttgaagagatagacttctccatggaagaatctatgattcatctgccaaataaagcttgttttctgatggcacacaatggatgggtaatgggagatgatcctcttcgaaactttgctgaaccgggttcagaagtttacctaaggagagaacttatttgctggggagacagtgttaaattacgctatgggaataaaccagaggactgtccttatctctgggcacacatgaaaaaatacactgaaataactgcaacttatttccagggagtacgtcttgataactgccactcaacacctcttcacgtagctgagtacatgttggatgctgctaggaatttgcaacccaatttatatgtagtagctgaactgttcacaggaagtgaagatctggacaatgtctttgttactagactgggcattagttccttaataagagaggcaatgagtgcatataatagtcatgaagagggcagattagtttaccgatatggaggagaacctgttggatcctttgttcagccctgtttgaggcctttaatgccagctattgcacatgccctgtttatggatattacgcatgataatgagtgtcctattgtgcatagatcagcgtatgatgctcttccaagtactacaattgtttctatggcatgttgtgctagtggaagtacaagaggctatgatgaattagtgcctcatcagatttcagtggtttctgaagaacggttttacactaagtggaatcctgaagcattgccttcaaacacaggtgaagttaatttccaaagcggcattattgcagccaggtgtgctatcagtaaacttcatcaggagcttggagccaagggttttattcaggtgtatgtggatcaagttgatgaagacatagtggcagtaacaagacactcacctagcatccatcagtctgttgtggctgtatctagaactgctttcaggaatcccaagacttcattttacagcaaggaagtgcctcaaatgtgcatccctggcaaaattgaagaagtagttcttgaagctagaactattgagagaaacacgaaaccttataggaaggatgagaattcaatcaatggaacaccagatatcacagtagaaattagagaacatattcagcttaatgaaagtaaaattgttaaacaagctggagttgccacaaaagggcccaatgaatatattcaagaaatagaatttgaaaacttgtctccaggaagtgttattatattcagagttagtcttgatccacatgcacaagtcgctgttggaattcttcgaaatcatctgacacaattcagtcctcactttaaatctggcagcctagctgttgacaatgcagatcctatattaaaaattccttttgcttctcttgcctccagattaactttggctgagctaaatcagatcctttaccgatgtgaatcagaagaaaaggaagatggtggagggtgctatgacataccaaactggtcagcccttaaatatgcaggtcttcaaggtttaatgtctgtattggcagaaataagaccaaagaatgacttggggcatcctttttgtaataatttgagatctggagattggatgattgactatgtcagtaaccggcttatttcacgatcaggaactattgctgaagttggtaaatggttgcaggctatgttcttctacctgaagcagatcccacgttaccttatcccatgttactttgatgctatattaattggtgcatataccactcttctggatacagcatggaagcagatgtcaagctttgttcagaatggttcaacctttgtgaaacacctttcattgggttcagttcaactgtgtggagtaggaaaattcccttccctgccaattctttcacctgccctaatggatgtaccttataggttaaatgagatcacaaaagaaaaggagcaatgttgtgtttctctagctgcaggcttacctcatttttcttctggtattttccgctgctggggaagggatacttttattgcacttagaggtatactgctgattactggacgctatgtagaagccaggaatattattttagcatttgcgggtaccctgaggcatggtctcattcctaatctactgggtgaaggaatttatgccagatacaattgtcgggatgctgtgtggtggtggctgcagtgtatccaggattactgtaaaatggttccaaatggtctagacattctcaagtgcccagtttccagaatgtatcctacagatgattctgctcctttgcctgctggcacactggatcagccattgtttgaagtcatacaggaagcaatgcaaaaacacatgcagggcatacagttccgagaaaggaatgctggtccccagatagatcgaaacatgaaggacgaaggttttaatataactgcaggagttgatgaagaaacaggatttgtttatggaggaaatcgtttcaattgtggcacatggatggataaaatgggagaaagtgacagagctagaaacagaggaatcccagccacaccaagagatgggtctgctgtggaaattgtgggcctgagtaaatctgctgttcgctggttgctggaattatccaaaaaaaatattttcccttatcatgaagtcacagtaaaaagacatggaaaggctataaaggtctcatatgatgagtggaacagaaaaatacaagacaactttgaaaagctatttcatgtttccgaagacccttcagatttaaatgaaaagcatccaaatctggttcacaaacgtggcatatacaaagatagttatggagcttcaagtccttggtgtgactatcagctcaggcctaattttaccatagcaatggttgtggcccctgagctctttactacagaaaaagcatggaaagctttggagattgcagaaaaaaaattgcttggtccccttggcatgaaaactttagatccagatgatatggtttactgtggaatttatgacaatgcattagacaatgacaactacaatcttgctaaaggtttcaattatcaccaaggacctgagtggctgtggcctattgggtattttcttcgtgcaaaattatatttttccagattgatgggcccggagactactgcaaagactatagttttggttaaaaatgttctttcccgacattatgttcatcttgagagatccccttggaaaggacttccagaactgaccaatgagaatgcccagtactgtcctttcagctgtgaaacacaagcctggtcaattgctactattcttgagacactttatgatttatagtttattacagatattaagtatgcaattacttgtattataggatgcaaggtcatcatatgtaaatgccttatatgcacaggctcaagttgttttaaaaatctcatttattataatattgatgctcaattaggtaagattgtaaaagcattgattttttttaatgtacagaggtagatttcaatttgaatcagaaagaaatatcattaccaatgaaatgtgtttgagttcagtaagaattattcaaatgcctagaaatccatagtttggaaaagaaaaatcatgtcatcttctatttgtacagaaatgaaaataaaatatgaaaataatgaaagaaatgaaaagatagcttttaattgtggtatatataatcttcagtaacaatacatactgaatacgctgtggttcattaatattaacaccacgtactatagtattcttaatacagtgctcactgcatttaataaatatttaataaatgatgaatgatagaagtttccatctacaatatatgttcctaaatggagcacagatgttcaaactatgctttcattttttcactgatatattaatttttgtgtaatgaatgccaacagtatattttatatgatttacttatgtgaggaaacatgcaaagcattaggaaatttatttcctaaaaacagttttgtaaaattagtattgagttctattgagtattataagatagcttacattttcaaaatggaaattgtcggtcatatttctagaactttaaagaaaaaagaatgttatattagttttctaaaactcaactatctttagtcatgttcaaaaatctattgctagatcatagtagatactggttttctattaactcaaaacctacattgacaagtttaacattgagaagaatcttaacaaaaatatggatatgaattcagtagatatcttaaattcaataaaatcactggaagtttttcatgataacttattttaagatgccttaaaaatcttaaagtcacaaaaggaaaaaggtttttaacatttacatgagttaacattttttcatagaacttatttcctagatagaattttttactgttttttactgttttcttaagaaaacagttaaatcattatgcattcagttggaagaaagtagtggcaagaattctttcattgctatataatattcagtggctcatttatacctaataaaataatggtattttaaaataatgctactttcaaagtagcatttttttagttagtttacaggttacatacccaaaaccttaactatgactaagaaattaaagaagaaaaccagcaaactaaaacttctgggcagcaaaaatatataaatgcttcagatgtcaaatacccatgcttgaaagctcgtgtaatttactttaagattatctgcctgctcttcttcaaagctgaccttgctttagaaatagttttaactagcttagttttctggtttccaaaactaaaatagattaaatcctacaaatttaaggacagttgtgacagtaatctgaccactatctataaatacattggacattggtttccaaatctccctttcttcttcagttccttccttgttcaatatatacccttctctaaactgtgcgggtaaaaggaatgactgtccttgagagaaccattagtttatcaaaggtttatgtagttttgttgctgtaccctaactttgatattcagggaggtaggaaaggtaacagaaaaccagcatatttaatcaaagcaagaagtaatcgctgacagttaaatgtgaccaaaaaaattaaaagttcacaatttttttaatgtagccatttggggttatctctagtaaggcagatacccacgttggtaaatttttaggatattgtgttgcactagaaaactaagtggttcatatttctaatgaggaagattaatgaaagaacattgttatattctgcgtggtatattttaaagtttaagaaggcatgttaaacattatttcctctatggtagttaaaatacagaattagatttttaacaggtgtcatttgactaaacgtttcggtagaatgcttcatacttgagtgatgctggataaggtattgtatttcaacaatggactatgccttggtttttcactaatcaaaatcaaaattactctttaacatgataaatgaatttaccagtttagtatgctgtggtattttaataagttttcaaagataattgggaaaacatgagactggtcatattgatgaatattgtaacatgtgaattgtgatccatttctgatatgtcttgaactactgtgtctagtgggcaaatgtcattgttacctctgtgtgttaagaaaataaaaatattttctaaaggtctgt

SEQ ID NO: 20 - DNA, 7449 bp, Homo sapiens
ctgtctacggcagctattccagaggcaacaactgcttccctctgttctcatctccccattggtggctggcgacccgaatttgggaatggggagattgcccacctgttatctttgagcagactaatctcttgtaagcagaagtgccattcggagtctccagagccctgtggcttggggctgggaatgtccccctgacttcaggctttcctaagtgtattgcttttctctgagaatggtctaggtttttaattttttaattgtaagaatctgtaatacagcatttttatttcggtcttattcgttgtgctcaaaggcaggaaacaactattaatttgccttctcgaatcttaatagttataagattcattctctttcattgctctgctaggcataaaacacacttcgaacatgggtaactcattcgactgtggagttcttttaattcttatgaaagatttcaaatcctctagaagccaaaatgggacacagtaaacagattcgaattttacttctgaacgaaatggagaaactggaaaagaccctcttcagacttgaacaagggtatgagctacagttccgattaggcccaactttacagggaaaagcagttaccgtgtatacaaattacccatttcctggagaaacatttaatagagaaaaattccgttctctggattgggaaaatccaacagaaagagaagatgattctgataaatactgtaaacttaatctgcaacaatctggttcatttcagtattatttccttcaaggaaatgagaaaagtggtggaggttacatagttgtggaccccattttacgtgttggtgctgataatcatgtgctacccttggactgtgttactcttcagacatttttagctaagtgtttgggaccttttgatgaatgggaaagcagacttagggttgcaaaagaatcaggctacaacatgattcattttaccccattgcagactcttggactatctaggtcatgctactcccttgccaatcagttagaattaaatcctgacttttcaagacctaatagaaagtatacctggaatgatgttggacagctagtggaaaaattaaaaaaggaatggaatgttatttgtattactgatgttgtctacaatcatactgctgctaatagtaaatggatccaggaacatccagaatgtgcctataatcttgtgaattctccacacttaaaacctgcctgggtcttagacagagcactttggcgtttctcctgtgatgttgcagaagggaaatacaaagaaaagggaatacctgctttgattgaaaatgatcaccatatgaattccatccgaaaaataatttgggaggatatttttccaaagcttaaactctgggaatttttccaagtagatgtcaacaaagcggttgagcaatttagaagacttcttacacaagaaaataggcgagtaaccaagtctgatccaaaccaacaccttacgattattcaagatcctgaatacagacggtttggctgtactgtagatatgaacattgcactaacgactttcataccacatgacaaggggccagcagcaattgaagaatgctgtaattggtttcataaaagaatggaggaattaaattcagagaagcatcgactcattaactatcatcaggaacaggcagttaattgccttttgggaaatgtgttttatgaacgactggctggccatggtccaaaactaggacctgtcactagaaagcatcctttagttaccaggtattttactttcccatttgaagagatagacttctccatggaagaatctatgattcatctgccaaataaagcttgttttctgatggcacacaatggatgggtaatgggagatgatcctcttcgaaactttgctgaaccgggttcagaagtttacctaaggagagaacttatttgctggggagacagtgttaaattacgctatgggaataaaccagaggactgtccttatctctgggcacacatgaaaaaatacactgaaataactgcaacttatttccagggagtacgtcttgataactgccactcaacacctcttcacgtagctgagtacatgttggatgctgctaggaatttgcaacccaatttatatgtagtagctgaactgttcacaggaagtgaagatctggacaatgtctttgttactagactgggcattagttccttaataagagaggcaatgagtgcatataatagtcatgaagagggcagattagtttaccgatatggaggagaacctgttggatcctttgttcagccctgtttgaggcctttaatgccagctattgcacatgccctgtttatggatattacgcatgataatgagtgtcctattgtgcatagatcagcgtatgatgctcttccaagtactacaattgtttctatggcatgttgtgctagtggaagtacaagaggctatgatgaattagtgcctcatcagatttcagtggtttctgaagaacggttttacactaagtggaatcctgaagcattgccttcaaacacaggtgaagttaatttccaaagcggcattattgcagccaggtgtgctatcagtaaacttcatcaggagcttggagccaagggttttattcaggtgtatgtggatcaagttgatgaagacatagtggcagtaacaagacactcacctagcatccatcagtctgttgtggctgtatctagaactgctttcaggaatcccaagacttcattttacagcaaggaagtgcctcaaatgtgcatccctggcaaaattgaagaagtagttcttgaagctagaactattgagagaaacacgaaaccttataggaaggatgagaattcaatcaatggaacaccagatatcacagtagaaattagagaacatattcagcttaatgaaagtaaaattgttaaacaagctggagttgccacaaaagggcccaatgaatatattcaagaaatagaatttgaaaacttgtctccaggaagtgttattatattcagagttagtcttgatccacatgcacaagtcgctgttggaattcttcgaaatcatctgacacaattcagtcctcactttaaatctggcagcctagctgttgacaatgcagatcctatattaaaaattccttttgcttctcttgcctccagattaactttggctgagctaaatcagatcctttaccgatgtgaatcagaagaaaaggaagatggtggagggtgctatgacataccaaactggtcagcccttaaatatgcaggtcttcaaggtttaatgtctgtattggcagaaataagaccaaagaatgacttggggcatcctttttgtaataatttgagatctggagattggatgattgactatgtcagtaaccggcttatttcacgatcaggaactattgctgaagttggtaaatggttgcaggctatgttcttctacctgaagcagatcccacgttaccttatcccatgttactttgatgctatattaattggtgcatataccactcttctggatacagcatggaagcagatgtcaagctttgttcagaatggttcaacctttgtgaaacacctttcattgggttcagttcaactgtgtggagtaggaaaattcccttccctgccaattctttcacctgccctaatggatgtaccttataggttaaatgagatcacaaaagaaaaggagcaatgttgtgtttctctagctgcaggcttacctcatttttcttctggtattttccgctgctggggaagggatacttttattgcacttagaggtatactgctgattactggacgctatgtagaagccaggaatattattttagcatttgcgggtaccctgaggcatggtctcattcctaatctactgggtgaaggaatttatgccagatacaattgtcgggatgctgtgtggtggtggctgcagtgtatccaggattactgtaaaatggttccaaatggtctagacattctcaagtgcccagtttccagaatgtatcctacagatgattctgctcctttgcctgctggcacactggatcagccattgtttgaagtcatacaggaagcaatgcaaaaacacatgcagggcatacagttccgagaaaggaatgctggtccccagatagatcgaaacatgaaggacgaaggttttaatataactgcaggagttgatgaagaaacaggatttgtttatggaggaaatcgtttcaattgtggcacatggatggataaaatgggagaaagtgacagagctagaaacagaggaatcccagccacaccaagagatgggtctgctgtggaaattgtgggcctgagtaaatctgctgttcgctggttgctggaattatccaaaaaaaatattttcccttatcatgaagtcacagtaaaaagacatggaaaggctataaaggtctcatatgatgagtggaacagaaaaatacaagacaactttgaaaagctatttcatgtttccgaagacccttcagatttaaatgaaaagcatccaaatctggttcacaaacgtggcatatacaaagatagttatggagcttcaagtccttggtgtgactatcagctcaggcctaattttaccatagcaatggttgtggcccctgagctctttactacagaaaaagcatggaaagctttggagattgcagaaaaaaaattgcttggtccccttggcatgaaaactttagatccagatgatatggtttactgtggaatttatgacaatgcattagacaatgacaactacaatcttgctaaaggtttcaattatcaccaaggacctgagtggctgtggcctattgggtattttcttcgtgcaaaattatatttttccagattgatgggcccggagactactgcaaagactatagttttggttaaaaatgttctttcccgacattatgttcatcttgagagatccccttggaaaggacttccagaactgaccaatgagaatgcccagtactgtcctttcagctgtgaaacacaagcctggtcaattgctactattcttgagacactttatgatttatagtttattacagatattaagtatgcaattacttgtattataggatgcaaggtcatcatatgtaaatgccttatatgcacaggctcaagttgttttaaaaatctcatttattataatattgatgctcaattaggtaagattgtaaaagcattgattttttttaatgtacagaggtagatttcaatttgaatcagaaagaaatatcattaccaatgaaatgtgtttgagttcagtaagaattattcaaatgcctagaaatccatagtttggaaaagaaaaatcatgtcatcttctatttgtacagaaatgaaaataaaatatgaaaataatgaaagaaatgaaaagatagcttttaattgtggtatatataatcttcagtaacaatacatactgaatacgctgtggttcattaatattaacaccacgtactatagtattcttaatacagtgctcactgcatttaataaatatttaataaatgatgaatgatagaagtttccatctacaatatatgttcctaaatggagcacagatgttcaaactatgctttcattttttcactgatatattaatttttgtgtaatgaatgccaacagtatattttatatgatttacttatgtgaggaaacatgcaaagcattaggaaatttatttcctaaaaacagttttgtaaaattagtattgagttctattgagtattataagatagcttacattttcaaaatggaaattgtcggtcatatttctagaactttaaagaaaaaagaatgttatattagttttctaaaactcaactatctttagtcatgttcaaaaatctattgctagatcatagtagatactggttttctattaactcaaaacctacattgacaagtttaacattgagaagaatcttaacaaaaatatggatatgaattcagtagatatcttaaattcaataaaatcactggaagtttttcatgataacttattttaagatgccttaaaaatcttaaagtcacaaaaggaaaaaggtttttaacatttacatgagttaacattttttcatagaacttatttcctagatagaattttttactgttttttactgttttcttaagaaaacagttaaatcattatgcattcagttggaagaaagtagtggcaagaattctttcattgctatataatattcagtggctcatttatacctaataaaataatggtattttaaaataatgctactttcaaagtagcatttttttagttagtttacaggttacatacccaaaaccttaactatgactaagaaattaaagaagaaaaccagcaaactaaaacttctgggcagcaaaaatatataaatgcttcagatgtcaaatacccatgcttgaaagctcgtgtaatttactttaagattatctgcctgctcttcttcaaagctgaccttgctttagaaatagttttaactagcttagttttctggtttccaaaactaaaatagattaaatcctacaaatttaaggacagttgtgacagtaatctgaccactatctataaatacattggacattggtttccaaatctccctttcttcttcagttccttccttgttcaatatatacccttctctaaactgtgcgggtaaaaggaatgactgtccttgagagaaccattagtttatcaaaggtttatgtagttttgttgctgtaccctaactttgatattcagggaggtaggaaaggtaacagaaaaccagcatatttaatcaaagcaagaagtaatcgctgacagttaaatgtgaccaaaaaaattaaaagttcacaatttttttaatgtagccatttggggttatctctagtaaggcagatacccacgttggtaaatttttaggatattgtgttgcactagaaaactaagtggttcatatttctaatgaggaagattaatgaaagaacattgttatattctgcgtggtatattttaaagtttaagaaggcatgttaaacattatttcctctatggtagttaaaatacagaattagatttttaacaggtgtcatttgactaaacgtttcggtagaatgcttcatacttgagtgatgctggataaggtattgtatttcaacaatggactatgccttggtttttcactaatcaaaatcaaaattactctttaacatgataaatgaatttaccagtttagtatgctgtggtattttaataagttttcaaagataattgggaaaacatgagactggtcatattgatgaatattgtaacatgtgaattgtgatccatttctgatatgtcttgaactactgtgtctagtgggcaaatgtcattgttacctctgtgtgttaagaaaataaaaatattttctaaaggtctgt

SEQ ID NO: 21 - DNA, 7182 bp, Homo sapiens
tgtataagaatttgcacatcccaagttgctatgtgaataggaatgcgtttccaggggaaggagaaagagacattacagagcagacagctctatgatgtttactatacttgctaaaatgtgaaattcagctaaattggaatacaaagtagtgccaaaacagcattaggtttgcggagttattttaaacataattgaaaaatcaaggttttttaatactttaaataaaacatctgtttttcaatgtggtaatttaagtcctacgatgagtttattaacatgtgctttttatttagggtatgagctacagttccgattaggcccaactttacagggaaaagcagttaccgtgtatacaaattacccatttcctggagaaacatttaatagagaaaaattccgttctctggattgggaaaatccaacagaaagagaagatgattctgataaatactgtaaacttaatctgcaacaatctggttcatttcagtattatttccttcaaggaaatgagaaaagtggtggaggttacatagttgtggaccccattttacgtgttggtgctgataatcatgtgctacccttggactgtgttactcttcagacatttttagctaagtgtttgggaccttttgatgaatgggaaagcagacttagggttgcaaaagaatcaggctacaacatgattcattttaccccattgcagactcttggactatctaggtcatgctactcccttgccaatcagttagaattaaatcctgacttttcaagacctaatagaaagtatacctggaatgatgttggacagctagtggaaaaattaaaaaaggaatggaatgttatttgtattactgatgttgtctacaatcatactgctgctaatagtaaatggatccaggaacatccagaatgtgcctataatcttgtgaattctccacacttaaaacctgcctgggtcttagacagagcactttggcgtttctcctgtgatgttgcagaagggaaatacaaagaaaagggaatacctgctttgattgaaaatgatcaccatatgaattccatccgaaaaataatttgggaggatatttttccaaagcttaaactctgggaatttttccaagtagatgtcaacaaagcggttgagcaatttagaagacttcttacacaagaaaataggcgagtaaccaagtctgatccaaaccaacaccttacgattattcaagatcctgaatacagacggtttggctgtactgtagatatgaacattgcactaacgactttcataccacatgacaaggggccagcagcaattgaagaatgctgtaattggtttcataaaagaatggaggaattaaattcagagaagcatcgactcattaactatcatcaggaacaggcagttaattgccttttgggaaatgtgttttatgaacgactggctggccatggtccaaaactaggacctgtcactagaaagcatcctttagttaccaggtattttactttcccatttgaagagatagacttctccatggaagaatctatgattcatctgccaaataaagcttgttttctgatggcacacaatggatgggtaatgggagatgatcctcttcgaaactttgctgaaccgggttcagaagtttacctaaggagagaacttatttgctggggagacagtgttaaattacgctatgggaataaaccagaggactgtccttatctctgggcacacatgaaaaaatacactgaaataactgcaacttatttccagggagtacgtcttgataactgccactcaacacctcttcacgtagctgagtacatgttggatgctgctaggaatttgcaacccaatttatatgtagtagctgaactgttcacaggaagtgaagatctggacaatgtctttgttactagactgggcattagttccttaataagagaggcaatgagtgcatataatagtcatgaagagggcagattagtttaccgatatggaggagaacctgttggatcctttgttcagccctgtttgaggcctttaatgccagctattgcacatgccctgtttatggatattacgcatgataatgagtgtcctattgtgcatagatcagcgtatgatgctcttccaagtactacaattgtttctatggcatgttgtgctagtggaagtacaagaggctatgatgaattagtgcctcatcagatttcagtggtttctgaagaacggttttacactaagtggaatcctgaagcattgccttcaaacacaggtgaagttaatttccaaagcggcattattgcagccaggtgtgctatcagtaaacttcatcaggagcttggagccaagggttttattcaggtgtatgtggatcaagttgatgaagacatagtggcagtaacaagacactcacctagcatccatcagtctgttgtggctgtatctagaactgctttcaggaatcccaagacttcattttacagcaaggaagtgcctcaaatgtgcatccctggcaaaattgaagaagtagttcttgaagctagaactattgagagaaacacgaaaccttataggaaggatgagaattcaatcaatggaacaccagatatcacagtagaaattagagaacatattcagcttaatgaaagtaaaattgttaaacaagctggagttgccacaaaagggcccaatgaatatattcaagaaatagaatttgaaaacttgtctccaggaagtgttattatattcagagttagtcttgatccacatgcacaagtcgctgttggaattcttcgaaatcatctgacacaattcagtcctcactttaaatctggcagcctagctgttgacaatgcagatcctatattaaaaattccttttgcttctcttgcctccagattaactttggctgagctaaatcagatcctttaccgatgtgaatcagaagaaaaggaagatggtggagggtgctatgacataccaaactggtcagcccttaaatatgcaggtcttcaaggtttaatgtctgtattggcagaaataagaccaaagaatgacttggggcatcctttttgtaataatttgagatctggagattggatgattgactatgtcagtaaccggcttatttcacgatcaggaactattgctgaagttggtaaatggttgcaggctatgttcttctacctgaagcagatcccacgttaccttatcccatgttactttgatgctatattaattggtgcatataccactcttctggatacagcatggaagcagatgtcaagctttgttcagaatggttcaacctttgtgaaacacctttcattgggttcagttcaactgtgtggagtaggaaaattcccttccctgccaattctttcacctgccctaatggatgtaccttataggttaaatgagatcacaaaagaaaaggagcaatgttgtgtttctctagctgcaggcttacctcatttttcttctggtattttccgctgctggggaagggatacttttattgcacttagaggtatactgctgattactggacgctatgtagaagccaggaatattattttagcatttgcgggtaccctgaggcatggtctcattcctaatctactgggtgaaggaatttatgccagatacaattgtcgggatgctgtgtggtggtggctgcagtgtatccaggattactgtaaaatggttccaaatggtctagacattctcaagtgcccagtttccagaatgtatcctacagatgattctgctcctttgcctgctggcacactggatcagccattgtttgaagtcatacaggaagcaatgcaaaaacacatgcagggcatacagttccgagaaaggaatgctggtccccagatagatcgaaacatgaaggacgaaggttttaatataactgcaggagttgatgaagaaacaggatttgtttatggaggaaatcgtttcaattgtggcacatggatggataaaatgggagaaagtgacagagctagaaacagaggaatcccagccacaccaagagatgggtctgctgtggaaattgtgggcctgagtaaatctgctgttcgctggttgctggaattatccaaaaaaaatattttcccttatcatgaagtcacagtaaaaagacatggaaaggctataaaggtctcatatgatgagtggaacagaaaaatacaagacaactttgaaaagctatttcatgtttccgaagacccttcagatttaaatgaaaagcatccaaatctggttcacaaacgtggcatatacaaagatagttatggagcttcaagtccttggtgtgactatcagctcaggcctaattttaccatagcaatggttgtggcccctgagctctttactacagaaaaagcatggaaagctttggagattgcagaaaaaaaattgcttggtccccttggcatgaaaactttagatccagatgatatggtttactgtggaatttatgacaatgcattagacaatgacaactacaatcttgctaaaggtttcaattatcaccaaggacctgagtggctgtggcctattgggtattttcttcgtgcaaaattatatttttccagattgatgggcccggagactactgcaaagactatagttttggttaaaaatgttctttcccgacattatgttcatcttgagagatccccttggaaaggacttccagaactgaccaatgagaatgcccagtactgtcctttcagctgtgaaacacaagcctggtcaattgctactattcttgagacactttatgatttatagtttattacagatattaagtatgcaattacttgtattataggatgcaaggtcatcatatgtaaatgccttatatgcacaggctcaagttgttttaaaaatctcatttattataatattgatgctcaattaggtaagattgtaaaagcattgattttttttaatgtacagaggtagatttcaatttgaatcagaaagaaatatcattaccaatgaaatgtgtttgagttcagtaagaattattcaaatgcctagaaatccatagtttggaaaagaaaaatcatgtcatcttctatttgtacagaaatgaaaataaaatatgaaaataatgaaagaaatgaaaagatagcttttaattgtggtatatataatcttcagtaacaatacatactgaatacgctgtggttcattaatattaacaccacgtactatagtattcttaatacagtgctcactgcatttaataaatatttaataaatgatgaatgatagaagtttccatctacaatatatgttcctaaatggagcacagatgttcaaactatgctttcattttttcactgatatattaatttttgtgtaatgaatgccaacagtatattttatatgatttacttatgtgaggaaacatgcaaagcattaggaaatttatttcctaaaaacagttttgtaaaattagtattgagttctattgagtattataagatagcttacattttcaaaatggaaattgtcggtcatatttctagaactttaaagaaaaaagaatgttatattagttttctaaaactcaactatctttagtcatgttcaaaaatctattgctagatcatagtagatactggttttctattaactcaaaacctacattgacaagtttaacattgagaagaatcttaacaaaaatatggatatgaattcagtagatatcttaaattcaataaaatcactggaagtttttcatgataacttattttaagatgccttaaaaatcttaaagtcacaaaaggaaaaaggtttttaacatttacatgagttaacattttttcatagaacttatttcctagatagaattttttactgttttttactgttttcttaagaaaacagttaaatcattatgcattcagttggaagaaagtagtggcaagaattctttcattgctatataatattcagtggctcatttatacctaataaaataatggtattttaaaataatgctactttcaaagtagcatttttttagttagtttacaggttacatacccaaaaccttaactatgactaagaaattaaagaagaaaaccagcaaactaaaacttctgggcagcaaaaatatataaatgcttcagatgtcaaatacccatgcttgaaagctcgtgtaatttactttaagattatctgcctgctcttcttcaaagctgaccttgctttagaaatagttttaactagcttagttttctggtttccaaaactaaaatagattaaatcctacaaatttaaggacagttgtgacagtaatctgaccactatctataaatacattggacattggtttccaaatctccctttcttcttcagttccttccttgttcaatatatacccttctctaaactgtgcgggtaaaaggaatgactgtccttgagagaaccattagtttatcaaaggtttatgtagttttgttgctgtaccctaactttgatattcagggaggtaggaaaggtaacagaaaaccagcatatttaatcaaagcaagaagtaatcgctgacagttaaatgtgaccaaaaaaattaaaagttcacaatttttttaatgtagccatttggggttatctctagtaaggcagatacccacgttggtaaatttttaggatattgtgttgcactagaaaactaagtggttcatatttctaatgaggaagattaatgaaagaacattgttatattctgcgtggtatattttaaagtttaagaaggcatgttaaacattatttcctctatggtagttaaaatacagaattagatttttaacaggtgtcatttgactaaacgtttcggtagaatgcttcatacttgagtgatgctggataaggtattgtatttcaacaatggactatgccttggtttttcactaatcaaaatcaaaattactctttaacatgataaatgaatttaccagtttagtatgctgtggtattttaataagttttcaaagataattgggaaaacatgagactggtcatattgatgaatattgtaacatgtgaattgtgatccatttctgatatgtcttgaactactgtgtctagtgggcaaatgtcattgttacctctgtgtgttaagaaaataaaaatattttctaaaggtctgt

SEQ ID NO: 22 - DNA, 7182 bp, Homo sapiens
gggtaactcattcgactgtggagttcttttaattcttatgaaagatttcaaatcctctagaagccaaaatgggacacagtaaacagattcgaattttacttctgaacgaaatggagaaactggaaaagaccctcttcagacttgaacaagaaactgggtctcactatgttgcccaggttgatattgaactcctggactcaagcaaccctccctctttggcctctgaaagtactgggattacaagcataagccaccgggcatggccccaattctgagcattaatttatttattgggtatgagctacagttccgattaggcccaactttacagggaaaagcagttaccgtgtatacaaattacccatttcctggagaaacatttaatagagaaaaattccgttctctggattgggaaaatccaacagaaagagaagatgattctgataaatactgtaaacttaatctgcaacaatctggttcatttcagtattatttccttcaaggaaatgagaaaagtggtggaggttacatagttgtggaccccattttacgtgttggtgctgataatcatgtgctacccttggactgtgttactcttcagacatttttagctaagtgtttgggaccttttgatgaatgggaaagcagacttagggttgcaaaagaatcaggctacaacatgattcattttaccccattgcagactcttggactatctaggtcatgctactcccttgccaatcagttagaattaaatcctgacttttcaagacctaatagaaagtatacctggaatgatgttggacagctagtggaaaaattaaaaaaggaatggaatgttatttgtattactgatgttgtctacaatcatactgctgctaatagtaaatggatccaggaacatccagaatgtgcctataatcttgtgaattctccacacttaaaacctgcctgggtcttagacagagcactttggcgtttctcctgtgatgttgcagaagggaaatacaaagaaaagggaatacctgctttgattgaaaatgatcaccatatgaattccatccgaaaaataatttgggaggatatttttccaaagcttaaactctgggaatttttccaagtagatgtcaacaaagcggttgagcaatttagaagacttcttacacaagaaaataggcgagtaaccaagtctgatccaaaccaacaccttacgattattcaagatcctgaatacagacggtttggctgtactgtagatatgaacattgcactaacgactttcataccacatgacaaggggccagcagcaattgaagaatgctgtaattggtttcataaaagaatggaggaattaaattcagagaagcatcgactcattaactatcatcaggaacaggcagttaattgccttttgggaaatgtgttttatgaacgactggctggccatggtccaaaactaggacctgtcactagaaagcatcctttagttaccaggtattttactttcccatttgaagagatagacttctccatggaagaatctatgattcatctgccaaataaagcttgttttctgatggcacacaatggatgggtaatgggagatgatcctcttcgaaactttgctgaaccgggttcagaagtttacctaaggagagaacttatttgctggggagacagtgttaaattacgctatgggaataaaccagaggactgtccttatctctgggcacacatgaaaaaatacactgaaataactgcaacttatttccagggagtacgtcttgataactgccactcaacacctcttcacgtagctgagtacatgttggatgctgctaggaatttgcaacccaatttatatgtagtagctgaactgttcacaggaagtgaagatctggacaatgtctttgttactagactgggcattagttccttaataagagaggcaatgagtgcatataatagtcatgaagagggcagattagtttaccgatatggaggagaacctgttggatcctttgttcagccctgtttgaggcctttaatgccagctattgcacatgccctgtttatggatattacgcatgataatgagtgtcctattgtgcatagatcagcgtatgatgctcttccaagtactacaattgtttctatggcatgttgtgctagtggaagtacaagaggctatgatgaattagtgcctcatcagatttcagtggtttctgaagaacggttttacactaagtggaatcctgaagcattgccttcaaacacaggtgaagttaatttccaaagcggcattattgcagccaggtgtgctatcagtaaacttcatcaggagcttggagccaagggttttattcaggtgtatgtggatcaagttgatgaagacatagtggcagtaacaagacactcacctagcatccatcagtctgttgtggctgtatctagaactgctttcaggaatcccaagacttcattttacagcaaggaagtgcctcaaatgtgcatccctggcaaaattgaagaagtagttcttgaagctagaactattgagagaaacacgaaaccttataggaaggatgagaattcaatcaatggaacaccagatatcacagtagaaattagagaacatattcagcttaatgaaagtaaaattgttaaacaagctggagttgccacaaaagggcccaatgaatatattcaagaaatagaatttgaaaacttgtctccaggaagtgttattatattcagagttagtcttgatccacatgcacaagtcgctgttggaattcttcgaaatcatctgacacaattcagtcctcactttaaatctggcagcctagctgttgacaatgcagatcctatattaaaaattccttttgcttctcttgcctccagattaactttggctgagctaaatcagatcctttaccgatgtgaatcagaagaaaaggaagatggtggagggtgctatgacataccaaactggtcagcccttaaatatgcaggtcttcaaggtttaatgtctgtattggcagaaataagaccaaagaatgacttggggcatcctttttgtaataatttgagatctggagattggatgattgactatgtcagtaaccggcttatttcacgatcaggaactattgctgaagttggtaaatggttgcaggctatgttcttctacctgaagcagatcccacgttaccttatcccatgttactttgatgctatattaattggtgcatataccactcttctggatacagcatggaagcagatgtcaagctttgttcagaatggttcaacctttgtgaaacacctttcattgggttcagttcaactgtgtggagtaggaaaattcccttccctgccaattctttcacctgccctaatggatgtaccttataggttaaatgagatcacaaaagaaaaggagcaatgttgtgtttctctagctgcaggcttacctcatttttcttctggtattttccgctgctggggaagggatacttttattgcacttagaggtatactgctgattactggacgctatgtagaagccaggaatattattttagcatttgcgggtaccctgaggcatggtctcattcctaatctactgggtgaaggaatttatgccagatacaattgtcgggatgctgtgtggtggtggctgcagtgtatccaggattactgtaaaatggttccaaatggtctagacattctcaagtgcccagtttccagaatgtatcctacagatgattctgctcctttgcctgctggcacactggatcagccattgtttgaagtcatacaggaagcaatgcaaaaacacatgcagggcatacagttccgagaaaggaatgctggtccccagatagatcgaaacatgaaggacgaaggttttaatataactgcaggagttgatgaagaaacaggatttgtttatggaggaaatcgtttcaattgtggcacatggatggataaaatgggagaaagtgacagagctagaaacagaggaatcccagccacaccaagagatgggtctgctgtggaaattgtgggcctgagtaaatctgctgttcgctggttgctggaattatccaaaaaaaatattttcccttatcatgaagtcacagtaaaaagacatggaaaggctataaaggtctcatatgatgagtggaacagaaaaatacaagacaactttgaaaagctatttcatgtttccgaagacccttcagatttaaatgaaaagcatccaaatctggttcacaaacgtggcatatacaaagatagttatggagcttcaagtccttggtgtgactatcagctcaggcctaattttaccatagcaatggttgtggcccctgagctctttactacagaaaaagcatggaaagctttggagattgcagaaaaaaaattgcttggtccccttggcatgaaaactttagatccagatgatatggtttactgtggaatttatgacaatgcattagacaatgacaactacaatcttgctaaaggtttcaattatcaccaaggacctgagtggctgtggcctattgggtattttcttcgtgcaaaattatatttttccagattgatgggcccggagactactgcaaagactatagttttggttaaaaatgttctttcccgacattatgttcatcttgagagatccccttggaaaggacttccagaactgaccaatgagaatgcccagtactgtcctttcagctgtgaaacacaagcctggtcaattgctactattcttgagacactttatgatttatagtttattacagatattaagtatgcaattacttgtattataggatgcaaggtcatcatatgtaaatgccttatatgcacaggctcaagttgttttaaaaatctcatttattataatattgatgctcaattaggtaagattgtaaaagcattgattttttttaatgtacagaggtagatttcaatttgaatcagaaagaaatatcattaccaatgaaatgtgtttgagttcagtaagaattattcaaatgcctagaaatccatagtttggaaaagaaaaatcatgtcatcttctatttgtacagaaatgaaaataaaatatgaaaataatgaaagaaatgaaaagatagcttttaattgtggtatatataatcttcagtaacaatacatactgaatacgctgtggttcattaatattaacaccacgtactatagtattcttaatacagtgctcactgcatttaataaatatttaataaatgatgaatgatagaagtttccatctacaatatatgttcctaaatggagcacagatgttcaaactatgctttcattttttcactgatatattaatttttgtgtaatgaatgccaacagtatattttatatgatttacttatgtgaggaaacatgcaaagcattaggaaatttatttcctaaaaacagttttgtaaaattagtattgagttctattgagtattataagatagcttacattttcaaaatggaaattgtcggtcatatttctagaactttaaagaaaaaagaatgttatattagttttctaaaactcaactatctttagtcatgttcaaaaatctattgctagatcatagtagatactggttttctattaactcaaaacctacattgacaagtttaacattgagaagaatcttaacaaaaatatggatatgaattcagtagatatcttaaattcaataaaatcactggaagtttttcatgataacttattttaagatgccttaaaaatcttaaagtcacaaaaggaaaaaggtttttaacatttacatgagttaacattttttcatagaacttatttcctagatagaattttttactgttttttactgttttcttaagaaaacagttaaatcattatgcattcagttggaagaaagtagtggcaagaattctttcattgctatataatattcagtggctcatttatacctaataaaataatggtattttaaaataatgctactttcaaagtagcatttttttagttagtttacaggttacatacccaaaaccttaactatgactaagaaattaaagaagaaaaccagcaaactaaaacttctgggcagcaaaaatatataaatgcttcagatgtcaaatacccatgcttgaaagctcgtgtaatttactttaagattatctgcctgctcttcttcaaagctgaccttgctttagaaatagttttaactagcttagttttctggtttccaaaactaaaatagattaaatcctacaaatttaaggacagttgtgacagtaatctgaccactatctataaatacattggacattggtttccaaatctccctttcttcttcagttccttccttgttcaatatatacccttctctaaactgtgcgggtaaaaggaatgactgtccttgagagaaccattagtttatcaaaggtttatgtagttttgttgctgtaccctaactttgatattcagggaggtaggaaaggtaacagaaaaccagcatatttaatcaaagcaagaagtaatcgctgacagttaaatgtgaccaaaaaaattaaaagttcacaatttttttaatgtagccatttggggttatctctagtaaggcagatacccacgttggtaaatttttaggatattgtgttgcactagaaaactaagtggttcatatttctaatgaggaagattaatgaaagaacattgttatattctgcgtggtatattttaaagtttaagaaggcatgttaaacattatttcctctatggtagttaaaatacagaattagatttttaacaggtgtcatttgactaaacgtttcggtagaatgcttcatacttgagtgatgctggataaggtattgtatttcaacaatggactatgccttggtttttcactaatcaaaatcaaaattactctttaacatgataaatgaatttaccagtttagtatgctgtggtattttaataagttttcaaagataattgggaaaacatgagactggtcatattgatgaatattgtaacatgtgaattgtgatccatttctgatatgtcttgaactactgtgtctagtgggcaaatgtcattgttacctctgtgtgttaagaaaataaaaatattttctaaaggtctgt

SEQ ID NO: 23 - protein, 6 aa, Artificial Sequence
  Description of Artificial Sequence Synthetic 6xHis tag
HHHHHH

SEQ ID NO: 24 - protein, 10 aa, Artificial Sequence
  Description of Artificial Sequence Synthetic peptide
EQKLISEEDL

SEQ ID NO: 25 - protein, 4 aa, Artificial Sequence
  Description of Artificial Sequence Synthetic peptide
AGIH

SEQ ID NO: 26 - protein, 5 aa, Artificial Sequence
  Description of Artificial Sequence Synthetic peptide
SAGIH

SEQ ID NO: 27 - protein, 8 aa, Artificial Sequence
  Description of Artificial Sequence Synthetic peptide
GFTFSNYG

SEQ ID NO: 28 - protein, 8 aa, Artificial Sequence
  Description of Artificial Sequence Synthetic peptide
ISSGSSTI

SEQ ID NO: 29 - protein, 9 aa, Artificial Sequence
  Description of Artificial Sequence Synthetic peptide
ARRGLLLDY

SEQ ID NO: 30 - protein, 10 aa, Artificial Sequence
  Description of Artificial Sequence Synthetic peptide
KSVSTSSYSY

SEQ ID NO: 31 - protein, 3 aa, Artificial Sequence
  Description of Artificial Sequence Synthetic peptide
YAS

SEQ ID NO: 32 - protein, 9 aa, Artificial Sequence
  Description of Artificial Sequence Synthetic peptide
QHSREFPWT

SEQ ID NO: 33 - protein, 50 aa, Artificial Sequence
  Description of Artificial Sequence Synthetic polypeptide
  misc_feature (1)..(50): This sequence may encompass 1-10 repeating “Gly Gly Gly Gly Ser” repeating units
GGGGSGGGGSGGGGSGGGGSGGGGSGGGGSGGGGSGGGGSGGGGSGGGGS

SEQ ID NO: 34 - protein, 7 aa, Artificial Sequence
  Description of Artificial Sequence Synthetic peptide
ASSLNIA

SEQ ID NO: 35 - protein, 7 aa, Artificial Sequence
  Description of Artificial Sequence Synthetic peptide
RRRRRRR

SEQ ID NO: 36 - protein, 5 aa, Artificial Sequence
  Description of Artificial Sequence Synthetic peptide
KFERQ

(19) United States
(12) Patent Application Publication
(10) Pub. No.: US 2018/0028676 A1
(43) Pub. Date: Feb. 1, 2018

(54) METHODS AND COMPOSITIONS FOR TREATMENT OF FORBES-CORI DISEASE

(71) Applicant: Valerion Therapeutics, LLC, Concord, MA (US)

(72) Inventor: Dustin D. Armstrong, Quincy, MA (US)

(21) Appl. No.: 15/612,800

(22) Filed: Jun. 2, 2017

Related U.S. Application Data
(63) Continuation of application No. 15/333,718, filed on Oct. 25, 2016, now abandoned.
Application No. 15/333,718 is a continuation of application No. 14/769,330, filed on Aug. 20, 2015, now abandoned.
(60) Provisional application No. 61/766,940, filed on Feb. 20, 2013.

(51) Int. Cl. A61K 38/45; C07K 16/28; C12N 9/10; C12N 9/34; C12N 9/44; A61K 38/47; C07K 16/44; A61K 38/00

(52) U.S. Cl. CPC A61K 47/64; C12Y 302/01033; C12Y 302/01003; A61K 38/45; A61K 38/47; C07K 16/28; C07K 16/44; C12N 9/1051; C12N 9/2428; C12N 9/2451; C07K 2319/10; C07K 2319/06; C07K 2319/00; C07K 2319/30; C07K 2319/33; A61K 38/00; C07K 2317/622; C07K 2317/77; C12Y 204/01025; C12Y 204/01125; C07K 2317/565

(57) ABSTRACT

In certain embodiments, the present disclosure provides compositions and methods for treating Forbes-Cori Disease.

### RELATED APPLICATIONS

This application claims the benefit of priority to U.S. provisional application 61/766,940, filed Feb. 20, 2013, which is hereby incorporated herein by reference in its entirety.

### SEQUENCE LISTING

The instant application contains a Sequence Listing which has been submitted electronically in ASCII format and is hereby incorporated by reference in its entirety. Said ASCII copy, created on Feb. 20, 2014, is named 106199-0010-WO1_SL.txt and is 130,924 bytes in size.

### BACKGROUND OF THE INVENTION

Forbes-Cori Disease, also known as Glycogen Storage Disease Type III or glycogen debrancher deficiency, is an autosomal recessive neuromuscular/hepatic disease with an estimated incidence of 1 in 100,000 births. Forbes-Cori Disease represents approximately 27% of all Glycogen Storage Disorders. The clinical picture in Forbes-Cori Disease is reasonably well established but exceptionally variable. Although generally considered a disease of the liver, with hepatomegaly and cirrhosis, Forbes-Cori Disease also is characterized by abnormalities in a variety of other systems. Muscle weakness, muscle wasting, hypoglycemia, dyslipidemia, and occasionally mental retardation also may be observed in this disease. Some patients possess facial abnormalities. Some patients also may be at an increased risk of osteoporosis. Different patients may suffer from one, or more than one, of these symptoms. The differences in clinical manifestations of this disease are often associated with different subtypes of this disease.

There are four subtypes of Forbes-Cori Disease. The Type A subtype accounts for approximately 80% of the cases, lacks enzymatic activity (e.g., both glucosidase and transferase activities associated with native enzymatic activity) and affects both the liver and muscle. The Type B subtype accounts for approximately 15% of the cases, lacks enzymatic activity (e.g., both glucosidase and transferase activities associated with native enzymatic activity) and affects only the liver. The Type C and D subtypes account for less than 5% of the cases, are associated with selective loss of glucosidase activity (Type C) or transferase activity (Type D) and are clinically similar to the Type A subtype.

Forbes-Cori Disease is caused by mutations in the AGL gene. The AGL gene encodes the amylo-1,6-glucosidase (AGL) protein, which is a cytoplasmic enzyme responsible for catalyzing the cleavage of terminal α-1,6-glucoside linkages in glycogen and similar molecules. The AGL protein has two separate enzymatic activities: 4-alpha-glucotransferase activity and amylo-1,6-glucosidase activity. Both catalytic activities are required for normal glycogen debranching activity. Glycogen is a highly branched polymer of glucose residues.

AGL is responsible for transferring three glucose subunits of glycogen from one parallel chain to another, thereby shortening one linear branch while lengthening another. Afterwards, the donator branch will still contain a single glucose residue with an alpha-1,6 linkage. The alpha-1,6 glucosidase of AGL will then remove that remaining residue, generating a “de-branched” form of that chain on the glycogen molecule. Without proper glycogen de-branching, as occurs in the absence of functional AGL, abnormal glycogens resembling an amylopectin-like structure (polyglucosan) result and accumulate in various tissues in the body, including hepatocytes and myocytes. This abnormal form of glycogen is typically insoluble and may be toxic to cells.

Currently, the primary treatment for Forbes-Cori is dietary and is aimed at maintaining normoglycemia (Ozen, et al., 2007, World J Gastroenterol, 13(18): 2545-46). To achieve this, patients are fed frequent meals high in carbohydrates and cornstarch supplements. Patients having myopathy are also fed a high-protein dict. Liver transplantation resolves all liver-related biochemical abnormalities, but the long-term effect of liver transplantation on myopathy/cardiomyopathy is unknown. (Ozen et al., 2007). These tools for managing Forbes-Cori are inadequate. Dietary regimens have significant compliance problems—particularly with young patients. As such, there is a need for a Forbes-Cori therapy that treats this disease's underlying causes, i.e., the patient's inability to break down glycogen, and that treats muscular and hepatic symptoms of this disease.

### SUMMARY OF THE INVENTION

There is a need in the art for methods and compositions for clearing cytoplasmic glycogen build-up in patients with Forbes-Cori disease. Such methods and compositions would improve treatment of Forbes-Cori disease. The present disclosure provides such methods and compositions. The methods and compositions provided herein can be used to replace functional AGL and/or to otherwise decrease deleterious glycogen build-up in the cytoplasm of cells, such as cells of the liver and muscle. Similarly, the methods and compositions provided herein can be used to improve deleterious symptoms of Forbes-Cori, for example, can be used to decrease levels of alanine transaminase, aspartate transaminase, alkaline phosphatase, and creatine phosphokinase (e.g., to decrease elevated levels of one or more such enzymes, such as in serum).

The disclosure provides a chimeric polypeptide comprising: (i) an amyloglucosidase (AGL) polypeptide, and (ii) an internalizing moiety. In certain embodiments, such a chimeric polypeptide comprises any one of the (i) AGL polypeptides described herein and any one of the (ii) internalizing moieties described herein. Such chimeric polypeptides have numerous uses, such as to evaluate delivery to the cytoplasm of cells in vitro and/or in vivo, to evaluate enzymatic activity, to increase enzymatic activity in a cell, or to identify a binding partner or substrate for AGL.

By way of example, in one aspect, the disclosure provides a chimeric polypeptide comprising: (i) an amyloglucosidase (AGL) polypeptide, and (ii) an internalizing moiety; wherein the chimeric polypeptide has amylo-1,6-glucosidase activity and 4-alpha-glucotransferase activity. In another aspect, the disclosure provides a chimeric polypeptide comprising: (i) an AGL polypeptide and (ii) an antibody or antigen binding fragment selected from: monoclonal antibody 3E10, or a variant thereof that retains cell penetrating activity, or a variant thereof that binds the same epitope as 3E10, or a variant thereof that binds DNA, or an antibody that has substantially the same cell penetrating activity as 3E10 and binds the same epitope as 3E10, or an antigen binding fragment of any of the foregoing; wherein the chimeric polypeptide has amylo-1,6-glucosidase activity and 4-alpha-glucotransferase activity.

In some embodiments, the internalizing moiety promotes delivery of the chimeric polypeptide into cells via an equilibrative nucleoside transporter (ENT) transporter. In some embodiments, the internalizing moiety promotes delivery of the chimeric polypeptide into cells via ENT2. In some embodiments, the internalizing moiety promotes delivery of said chimeric polypeptide into muscle cells. In some embodiments, the internalizing moiety promotes delivery of said chimeric polypeptide into one or more of muscle cells, hepatocytes and fibroblasts. It should be noted that when an internalizing moiety is described as promoting delivery into muscle cells, that does not imply that delivery is exclusive to muscle cells. All that is implied is that delivery is somewhat enriched to muscle cells versus one or more other cell types and that transit into cells is not ubiquitous across all cell types.

In some embodiments, the AGL polypeptide comprises an amino acid sequence at least 90% identical to any of SEQ ID NOs: 1, 2 or 3, and wherein the chimeric polypeptide has amylo-1,6-glucosidase activity and 4-alpha-glucotransferase activity. In some embodiments, the AGL polypeptide comprises an amino acid sequence at least 95% identical to any of SEQ ID NOs: 1, 2 or 3, and wherein the chimeric polypeptide has amylo-1,6-glucosidase activity and 4-alpha-glucotransferase activity. In some embodiments, the AGL polypeptide comprises an amino acid sequence identical to any of SEQ ID NOs: 1, 2 or 3, in the presence or absence of the N-terminal methionine, and wherein the chimeric polypeptide has amylo-1,6-glucosidase activity and 4-alpha-glucotransferase activity.

In some embodiments, the AGL polypeptide is a full length or substantially full length polypeptide. In some embodiments, the AGL polypeptide is a functional fragment of at least 500, at least 700, at least 750, at least 800, at least 900, at least 1000, at least 1200, at least 1300, or at least 1400 amino acids, and which functional fragment has amylo-1,6-glucosidase activity and 4-alpha-glucotransferase activity.

In some embodiments, the chimeric polypeptide further comprises one or more polypeptide portions that enhance one or more of in vivo stability, in vivo half life, uptake/administration, or purification. In some embodiments, the chimeric polypeptide lacks one or more N-glycosylation groups present in a wildtype AGL polypeptide. In some embodiments, the chimeric polypeptide lacks one or more O-glycosylation groups present in a wildtype AGL polypeptide. In some embodiments, the asparagine at any one of, or combination of, the amino acid positions corresponding to amino acid positions 69, 219, 797, 813, 839, 927, 1032, 1236 and 1380 of SEQ ID NO: 1 is substituted or deleted in said AGL polypeptide. In some embodiments, the serine at any one of, or combination of, the amino acid positions corresponding to amino acid positions 815, 841, 929 and 1034 of SEQ ID NO: 1 is substituted or deleted in said AGL polypeptide. In some embodiments, the threonine at any one of, or combination of, the amino acid positions corresponding to amino acid positions 71, 221, 799, 1238 and 1382 of SEQ ID NO: 1 is substituted or deleted in said AGL polypeptide. In some embodiments, the amino acid present at the amino acid position corresponding to any one of, or combination of, amino acid positions 220, 798, 814, 840, 928, 1033, 1237 and 1381 of SEQ ID NO: 1 is replaced with a proline in said AGL polypeptide.

In some embodiments, the internalizing moiety comprises an antibody or antigen binding fragment. In some embodiments, the antibody is a monoclonal antibody or fragment thereof. In some embodiments, the antibody is monoclonal antibody 3E10, or an antigen binding fragment thereof. In some embodiments, the internalizing moiety comprises a homing peptide. In some embodiments, the AGL polypeptide is chemically conjugated to the internalizing moiety. In some embodiments, the chimeric polypeptide is a fusion protein comprising the AGL polypeptide and the internalizing moiety. In some embodiments, the internalizing moiety transits cellular membranes via an equilibrative nucleoside transporter 2 (ENT2) transporter. In some embodiments, the antibody or antigen binding fragment is selected from: a monoclonal antibody 3E10, or a variant thereof that retains cell penetrating activity, or a variant thereof that binds the same epitope as 3E10, or an antibody that has substantially the same cell penetrating activity as 3E10 and binds the same epitope as 3E10, or an antigen binding fragment of any of the foregoing. In some embodiments, the antibody or antigen binding fragment is monoclonal antibody 3E10, or a variant thereof that retains cell penetrating activity, or an antigen binding fragment of 3E10 or said 3E10 variant. In some embodiments, the antibody or antigen binding fragment is a chimeric, humanized, or fully human antibody or antigen binding fragment. In some embodiments, the antibody or antigen binding fragment comprises a heavy chain variable domain comprising an amino acid sequence at least 95% identical to SEQ ID NO: 6, or a humanized variant thereof. In some embodiments, the antibody or antigen binding fragment comprises a light chain variable domain comprising an amino acid sequence at least 95% identical to SEQ ID NO: 8, or a humanized variant thereof. In some embodiments, the antibody or antigen binding fragment comprises a heavy chain variable domain comprising the amino acid sequence of SEQ ID NO: 6 and a light chain variable domain comprising the amino acid sequence of SEQ ID NO: 8, or a humanized variant thereof. In some embodiments, the antibody or antigen binding fragment comprises

a VH CDR1 having the amino acid sequence of SEQ ID NO: 9;

a VH CDR2 having the amino acid sequence of SEQ ID NO: 10;

a VH CDR3 having the amino acid sequence of SEQ ID NO: 11;

a VL CDR1 having the amino acid sequence of SEQ ID NO: 12;

a VL CDR2 having the amino acid sequence of SEQ ID NO: 13; and

a VL CDR3 having the amino acid sequence of SEQ ID NO: 14.

In some embodiments, the chimeric polypeptide is produced recombinantly to recombinantly conjugate the AGL polypeptide to the internalizing moiety. In some embodiments, the chimeric polypeptide is produced in a prokaryotic or eukaryotic cell. In some embodiments, the eukaryotic cell is selected from a yeast cell, an avian cell, an insect cell, or a mammalian cell. In some embodiments, the prokaryotic cell is bacterial cell.

In some embodiments, the chimeric polypeptide is a fusion protein. In some embodiments, the fusion protein comprises a linker. In some embodiments, the conjugate comprises a linker. In some embodiments, the linker conjugates or joins the AGL polypeptide to the internalizing moiety. In some embodiments, the conjugate does not include a linker, and the AGL polypeptide is conjugated or joined directly to the internalizing moiety. In some embodiments, the linker is a cleavable linker. In some embodiments, the internalizing moiety is conjugated or joined, directly or indirectly, to the N-terminal or C-terminal amino acid of the AGL polypeptide. In some embodiments, the internalizing moiety is conjugated or joined, directly or indirectly to an internal amino acid of the AGL polypeptide.

The present disclosure provides chimeric polypeptides comprising an AGL portion and an internalizing moiety portion. Any such chimeric polypeptide described herein as having any of the features of an AGL portion and any of the features of an internalizing moiety portion may be referred to as a “chimeric polypeptide of the disclosure” or an “AGL chimeric polypeptide” or an “AGL chimeric polypeptide of the disclosure”. In certain embodiments, the chimeric polypeptide has amylo-1,6-glucosidase activity and 4-alpha-glucotransferase activity.

In another aspect, the disclosure provides a nucleic acid construct, comprising a nucleotide sequence that encodes any of the chimeric polypeptides described above as a fusion protein. The disclosure also provides a nucleic acid construct, comprising a nucleotide sequence that encodes an AGL polypeptide, operably linked to a nucleotide sequence that encodes an internalizing moiety, wherein the nucleic acid construct encodes a chimeric polypeptide having AGL enzymatic activity and having the internalizing activity of the internalizing moiety. In some embodiments, the nucleotide sequence that encodes the AGL polypeptide encodes an AGL polypeptide comprising an amino acid sequence at least 90% identical to any of SEQ ID NOs: 1, 2 and 3. In some embodiments, the nucleotide sequence that encodes the AGL polypeptide encodes an AGL polypeptide comprising an amino acid sequence at least 95% identical to any of SEQ ID NOs: 1, 2 and 3. In some embodiments, the nucleotide sequence that encodes the AGL polypeptide encodes an AGL polypeptide comprising an amino acid sequence at least 98% identical to any of SEQ ID NO: 1, 2 and 3. In some embodiments, the nucleotide sequence that encodes an AGL polypeptide comprises SEQ ID NO: 17, 18, 19, or 20. In some embodiments, the nucleotide sequence that encodes an AGL polypeptide comprises SEQ ID NO: 21 or 22. In some embodiments, the nucleic acid construct further comprises a nucleotide sequence that encodes a linker. In some embodiments, the nucleic acid construct encodes an internalizing moiety, wherein the internalizing moiety is any of the antibodies or antigen-binding fragments disclosed herein.

In another aspect, the disclosure provides a composition comprising any of the chimeric polypeptides disclosed herein, and a pharmaceutically acceptable carrier. In some embodiments, the composition is substantially pyrogen-free.

In another aspect, the disclosure provides a method of treating Forbes-Cori disease in a subject in need thereof, comprising administering to the subject an effective amount of a chimeric polypeptide comprising: (i) an AGL polypeptide, and (ii) an internalizing moiety; wherein the chimeric polypeptide has amylo-1,6-glucosidase activity and 4-alpha-glucotransferase activity. In some embodiments, the method of treating Forbes-Cori disease in a subject in need thereof, comprises administering to the subject an effective amount of any of the chimeric polypeptide, nucleic acid construct, or compositions disclosed herein.

In another aspect, the disclosure provides a method of increasing glycogen debrancher enzyme activity in a cell, comprising contacting the cell with a chimeric polypeptide comprising: (i) an AGL polypeptide, and (ii) an internalizing moiety; wherein the chimeric polypeptide has amylo-1,6-glucosidase activity and 4-alpha-glucotransferase activity. In some embodiments, the internalizing moiety promotes delivery of the chimeric polypeptide into cells via an ENT transporter. In some embodiments, the cell is a cell in a subject in need thereof. In some embodiments, the subject in need thereof has hepatic symptoms associated with Forbes-Cori disease. In some embodiments, the subject in need thereof has neuromuscular symptoms associated with Forbes-Cori disease. In some embodiments the internalizing moiety promotes delivery of said chimeric polypeptide into muscle cells. In some embodiments, the internalizing moiety promotes delivery of said chimeric polypeptide into one or more of muscle cells, hepatocytes and fibroblasts. In some embodiments, the AGL polypeptide of the chimeric polypeptide for use in the methods disclosed herein is any of the AGL polypeptides described herein. In some embodiments, the internalizing moiety for use in the methods disclosed herein is any of the antibodies or antigen-binding fragments disclosed herein. In some embodiments, the internalizing moiety is conjugated to the AGL polypeptide by a linker. In some embodiments, the linker is cleavable. In other embodiments, the internalizing moiety is conjugated or joined directly to the AGL polypeptide.

In another aspect, the disclosure provides a use of any of the chimeric polypeptides disclosed herein in the manufacture of a medicament for treating Forbes-Cori disease. In another aspect, the disclosure provides any of the chimeric polypeptide disclosed herein for treating Forbes-Cori disease. In another aspect, the disclosure provides any of the chimeric polypeptides disclosed herein for delivery of said chimeric polypeptide into one or both of muscle cells and liver cells. In another aspect, the disclosure provides the use of any of the chimeric polypeptides disclosed herein in the manufacture of a medicament for delivery into one or both of muscle cells and liver cells.

In another aspect, the disclosure provides a use of any of the nucleic acid constructs disclosed herein in the manufacture of a medicament for treating Forbes-Cori disease. In some embodiments, the disclosure provides any of the nucleic acid constructs disclosed herein for treating Forbes-Cori disease.

In another aspect, the disclosure provides any of the compositions disclosed herein for use in treating Forbes-Cori disease.

In another aspect, the disclosure provides a method of delivering a chimeric polypeptide into a cell via an equilibrative nucleoside transporter (ENT2) pathway, comprising contacting a cell with a chimeric polypeptide, which chimeric polypeptide comprises (i) an AGL polypeptide, and (ii) an internalizing moiety that penetrates cells via ENT2; wherein the chimeric polypeptide has amylo-1,6-glucosidase activity and 4-alpha-glucotransferase activity. In some embodiments, the AGL polypeptide of the chimeric polypeptide for use in the methods disclosed herein is any of the AGL polypeptides described herein. In some embodiments, the internalizing moiety for use in the methods disclosed herein is any of the internalizing moieties disclosed herein. In some embodiments, the internalizing moiety is any of the antibodies or antigen-binding fragments disclosed herein. In some embodiments, the internalizing moiety promotes delivery of the chimeric polypeptide into cells. In some embodiments, the cell is a muscle cell, and the internalizing moiety promotes delivery of said chimeric polypeptide into muscle cells.

In another aspect, the disclosure provides a method of delivering a chimeric polypeptide into a muscle cell, comprising contacting a muscle cell with a chimeric polypeptide, which chimeric polypeptide comprises (i) an AGL polypeptide, and (ii) an internalizing moiety which promotes transport into muscle cells, wherein the internalizing moiety promotes transport of the chimeric polypeptide into cells, and wherein the chimeric polypeptide has amylo-1,6-glucosidase activity and 4-alpha-glucotransferase activity. In some embodiments, the AGL polypeptide of the chimeric polypeptide for use in the methods disclosed herein is any of the AGL polypeptides described herein. In some embodiments, the internalizing moiety for use in the methods disclosed herein is any of the internalizing moieties disclosed herein. In some embodiments, the internalizing moiety is any of the antibodies or antigen-binding fragments disclosed herein.

In another aspect, the disclosure provides a method of delivering a chimeric polypeptide into a hepatocyte, comprising contacting a hepatocyte with a chimeric polypeptide, which chimeric polypeptide comprises (i) an AGL polypeptide or functional fragment thereof, and (ii) an internalizing moiety; wherein the chimeric polypeptide has amylo-1,6-glucosidase activity and 4-alpha-glucotransferase activity. In some embodiments, the AGL polypeptide of the chimeric polypeptide for use in the methods disclosed herein is any of the AGL polypeptides described herein. In some embodiments, the internalizing moiety for use in the methods disclosed herein is any of the internalizing moieties disclosed herein. In some embodiments, the internalizing moiety is any of the antibodies or antigen-binding fragments disclosed herein.

In another aspect, the disclosure provides a method of increasing amyloglucosidase (AGL) enzymatic activity in a muscle cell, comprising contacting a muscle cell with a chimeric polypeptide, which chimeric polypeptide comprises (i) an AGL polypeptide, and (ii) an internalizing moiety; wherein the internalizing moiety promotes transport of the chimeric polypeptide into cells, and wherein the chimeric polypeptide has amylo-1,6-glucosidase activity and 4-alpha-glucotransferase activity. In some embodiments, the AGL polypeptide of the chimeric polypeptide for use in the methods disclosed herein is any of the AGL polypeptides described herein. In some embodiments, the internalizing moiety for use in the methods disclosed herein is any of the internalizing moieties disclosed herein. In some embodiments, the internalizing moiety is any of the antibodies or antigen-binding fragments disclosed herein.

In another aspect, the disclosure provides a method of increasing amyloglucosidase (AGL) enzymatic activity in a hepatocyte, comprising contacting a hepatocyte with a chimeric polypeptide, which chimeric polypeptide comprises (i) an AGL polypeptide or functional fragment thereof and (ii) an internalizing moiety; wherein the chimeric polypeptide has amylo-1,6-glucosidase activity and 4-alpha-glucotransferase activity. In some embodiments, the AGL polypeptide of the chimeric polypeptide for use in the methods disclosed herein is any of the AGL polypeptides described herein. In some embodiments, the internalizing moiety for use in the methods disclosed herein is any of the internalizing moieties disclosed herein. In some embodiments, the internalizing moiety is any of the antibodies or antigen-binding fragments disclosed herein.

For any of the foregoing, in certain embodiments, administering an AGL chimeric polypeptide of the disclosure, such as to cells or subjects in need thereof may be useful for treating (improving one or more symptoms of) Forbes-Cori Disease. In certain embodiments, administering an AGL chimeric polypeptide may have any one or more of the following affects: decrease accumulation of glycogen in cytoplasm of cells, decrease accumulation of glycogen in cytoplasm of muscle cells, decrease accumulation of glycogen in cytoplasm of liver, decrease elevated levels of alanine transaminase (such as elevated levels in serum), decrease elevated levels of aspartate transaminase (such as elevated levels in serum), decrease elevated levels of alkaline phosphatase (such as elevated levels in serum), and/or decrease elevated levels of creatine phosphokinase (such as elevated levels in serum). It should be noted that any of the AGL chimeric polypeptides described above or herein may be used in any of the methods described herein.

In another aspect, the disclosure provides a method of treating Forbes-Cori disease in a subject in need thereof, comprising contacting the cell with a chimeric polypeptide comprising: (i) a mature acid alpha-glucosidase (GAA) polypeptide and (ii) an internalizing moiety that promotes delivery into cells; wherein the chimeric polypeptide has acid alpha-glucosidase activity, and wherein the chimeric polypeptide does not comprise a GAA precursor polypeptide of approximately 110 kilodaltons (e.g., does not comprise residues 1-27 or 1-56 of GAA precursor polypeptide). The use of such chimeric polypeptides may be referred to herein as the use of GAA chimeric polypeptides of the disclosures. Similarly, such polypeptides may be referred to as GAA chimeric polypeptides of the disclosure.

In another aspect, the disclosure provides a method of decreasing glycogen accumulation in cytoplasm of cells of a Forbes-Cori patient, comprising contacting muscle cells with a chimeric polypeptide, which chimeric polypeptide comprises (i) a mature acid alpha-glucosidase (GAA) polypeptide and (ii) an internalizing moiety that promotes transport into cytoplasm of cells; wherein the chimeric polypeptide has acid alpha-glucosidase activity, and wherein the chimeric polypeptide does not comprise a GAA precursor polypeptide of approximately 110 kilodaltons.

In another aspect, the disclosure provides a method of increasing GAA activity in the cytoplasm of a cell, comprising delivering a chimeric polypeptide, wherein said chimeric polypeptide comprises: (i) a mature acid alpha-glucosidase (GAA) polypeptide and (ii) an internalizing moiety that promotes transport into cytoplasm of cells; wherein the chimeric polypeptide has acid alpha-glucosidase activity, and wherein the chimeric polypeptide does not comprise a GAA precursor polypeptide of approximately 110 kilodaltons. In some embodiments, the cell is in a subject, wherein said subject has Forbes-Cori disease, and contacting the cell comprises administering the GAA chimeric polypeptide to the patient via a route of delivery. In some embodiments, the subject in need thereof is a subject having pathologic cytoplasmic glycogen accumulation prior to initiation of treatment with said chimeric polypeptide. In some embodiments, the method is an in vitro method, and the cell is in culture. In some embodiments, the mature GAA polypeptide has a molecular weight of approximately 70-76 kilodaltons. In some embodiments, the mature GAA polypeptide consists of an amino acid sequence selected from residues 122-782 of SEQ ID NO: 4 or residues 204-782 of SEQ ID NO: 5. In some embodiments, the mature GAA polypeptide has a molecular weight of approximately 70-76 kilodaltons. In some embodiments, the mature GAA polypeptide has a molecular weight of approximately 70 kilodaltons. In some embodiments, the mature GAA polypeptide has a molecular weight of approximately 76 kilodaltons. In some embodiments, the mature GAA polypeptide is glycosylated. In other embodiments, the mature GAA polypeptide is not glycosylated. In some embodiments, the mature GAA polypeptide has a glycosylation pattern that differs from that of naturally occurring human GAA.

In some embodiments, the chimeric polypeptide comprising the mature GAA polypeptide reduces cytoplasmic glycogen accumulation.

In some embodiments, the chimeric polypeptide comprising the mature GAA polypeptide comprises any of the internalizing moieties disclosed herein. In some embodiments, the fusion protein comprises a linker. In some embodiments, the conjugate comprises a linker. In some embodiments, the linker conjugates or joins the AGL polypeptide to the internalizing moiety. In some embodiments, the conjugate does not include a linker, and the AGL polypeptide is conjugated or joined directly to the internalizing moiety. In some embodiments, the linker is a cleavable linker.

In some embodiments of any of the methods disclosed herein for administering any of the chimeric polypeptides disclosed herein (e.g., an AGL chimeric polypeptide or a GAA chimeric polypepde) to a subject, for example, a Forbes-Cori patient, the chimeric polypeptide is formulated with a pharmaceutically acceptable carrier. In some embodiments, the chimeric polypeptide is administered systemically. In some embodiments, the chimeric polypeptide is administered locally. In some embodiments, administered locally comprises administering via the hepatic portal vein. In some embodiments, the chimeric polypeptide is administered intravenously.

In another aspect, the disclosure provides GAA chimeric polypeptides, such as any of the GAA chimeric polypeptides described for use in treating Forbes-Cori Disease. In certain embodiments, administering a GAA chimeric polypeptide may have any one or more of the following affects: decrease accumulation of glycogen in cytoplasm of cells, decrease accumulation of glycogen in cytoplasm of muscle cells, decrease accumulation of glycogen in cytoplasm of liver, decrease elevated levels of alanine transaminase (such as elevated levels in serum), decrease elevated levels of aspartate transaminase (such as elevated levels in serum), decrease elevated levels of alkaline phosphatase (such as elevated levels in serum), and/or decrease elevated levels of creatine phosphokinase (such as elevated levels in serum). It should be noted that any of the GAA chimeric polypeptides described above or herein may be used in any of the methods described herein.

The disclosure contemplates that any one or more of the aspects and embodiments of the disclosure detailed above can be combined with each other and/or with any of the features disclosed below. Moreover, any one or more of the features of the disclosure described below may be combined.

### DETAILED DESCRIPTION OF THE INVENTION

The glycogen debranching enzyme (gene, AGL) amyloglucosidase (AGL) is a bifunctional enzyme that has two independent catalytic activities: oligo-1,4-1,4-glucotransferase activity and amylo-1,6-glucosidase activity. These independent catalytic activities occur at separate sites on the same polypeptide chain. AGL is a large monomeric protein having a molecular mass of 160-175 kDa. See, e.g., Shen et al., 2002, Curr Mol Med, 2:167-175; and Chen, 1987, Am. J. Hum. Genet., 41(6): 1002-15. Six different mRNA transcript variants of AGL exist in humans encoding three different AGL isoforms. These transcript variants differ in their 5′ untranslated region and tissue distribution. AGL-transcript variant 1 (SEQ ID NO: 17) is expressed in every tissue type examined (including liver and muscle), and transcript variants 2-4 (SEQ ID NOs: 18-20) are specifically expressed in skeletal muscle and heart. Transcript variants 5 and 6 (SEQ ID NOs: 21-22) are minor isoforms. See, e.g., Shen et al., 2002, Curr Mol Med, 2:167-175. AGL transcript variants 1-4 encode AGL isoform 1 (SEQ ID NO: 1), AGL transcript variant 5 encodes AGL isoform 2 (SEQ ID NO: 2), and AGL transcript variant 6 encodes AGL isoform 3 (SEQ ID NO: 3).

The acid alpha glucosidase enzyme (GAA) is an enzyme essential for the degradation of glycogen to glucose in lysosomes. Several isoforms of GAA exist (see, e.g., SEQ ID NOs: 4 and 5). The GAA enzyme is synthesized as a catalytically active, immature 110-kDa precursor that is glycosylated and modified in the Golgi by the addition of mannose 6-phosphate residues (M6P). See, e.g., Raben et al., 2006, Molecular Therapy 11, 48-56.

Forbes-Cori Disease is caused by mutations in the AGL gene The AGL gene encodes the AGL protein, which collaborates with phosphorylase to degrade glycogen in the cytoplasm. The two catalytic activities of AGL protein are a transferase activity (4-alpha-glucotransferase) and a glucosidase activity (amylo-alpha 1,6-glucosidase). Glycogen is a highly branched polymer of glucose residues. When glycogen is broken down by the body to produce energy, glucose molecules are removed from the glycogen chains. Without proper glycogen debranching, as occurs in the absence of functional AGL, glycogen begins to accumulate in cells throughout the body, including hepatocytes and myocytes. The accumulation of glycogen may be toxic to cells, and the absence of free glucose from the accumulated glycogen can result in a reduced energy supply for cells.

Without being bound by theory, administration of the AGL chimeric polypeptides described herein to a Forbes-Cori patient will replace or supplement the missing or low levels of endogenous AGL protein in the patient, thereby alleviating some or all of the symptoms associated with glycogen accumulation in the patient's cells. Without being bound by theory, the internalizing moiety will help promote delivery into some of the tissues most severely affected in Forbes-Cori disease patients, e.g. muscle or liver, and deliver the AGL protein to these tissues to help reverse or prevent further accumulation of glycogen in these tissues. In addition, one of the results of high glycogen deposition in liver and muscle is high and increasing levels of alanine transaminase, aspartate transaminase, alkaline phosphatase, and creatine phosphokinase—particularly in serum. Administration of an AGL chimeric polypeptide of the disclosure can be used to decrease the abnormally high levels of these enzymes observed in patients.

In a recent study, it was demonstrated that administration of GAA to Forbes-Cori cells resulted in a reduction in overall levels of glycogen in these cells. See, published US patent application US 20110104187. However, the GAA polypeptide used in this study was the full-length, immature precursor GAA polypeptide, and the activity of the full-length GAA polypeptide was limited primarily to lyosomes (see, US 20110104187). In addition, while it has been demonstrated that mature GAA polypeptides are more active than then the immature precursor and promote enhanced glycogen clearance as compared to the precursor GAA (Bijvoet, et al., 1998, Hum Mol Genet, 7(11): 1815-24), the mature form of GAA is poorly internalized by cells (Bijvoet et al., 1998). In addition, while mature GAA is a lysosomal protein that has optimal activity at lower pHs, mature GAA retains approximately 40% activity at neutral pH (i.e., the pH of the cytoplasm) (Martin-Touaux et al., 2002, Hum Mol Genet, 11(14): 1637-45). Until the present disclosure, there has been no guidance in the art as to how the more active mature GAA polypeptide could be administered to Forbes-Cori patients such that the mature GAA would reach the tissues and compartments that need it most, e.g., the cytoplasm of muscle and liver cells. Administration of any of the chimeric polypeptides disclosed herein comprising mature GAA and an internalizing moiety to a patient would ensure that mature GAA reached tissues such as muscle and liver and that the mature GAA activity was not limited to the lysosome. Without being bound by theory, the administered mature GAA polypeptide will replace the glucosidase activity of the missing or reduced levels of the AGL protein in the Forbes-Cori patient, thereby alleviating some or all of the symptoms associated with glycogen accumulation in the patient's cells. For example, one of the results of high glycogen deposition in liver and muscle is high and increasing levels of alanine transaminase, aspartate transaminase, alkaline phosphatase, and creatine phosphokinase—particularly in serum. Administration of a GAA chimeric polypeptide of the disclosure can be used to decrease the abnormally high levels of one or more of these enzymes observed in patients. As detailed herein, such reduction of these elevated enzyme levels may also be reduced following administration of AGL chimeric polypeptides of the disclosure.

In certain aspects, the disclosure provides using either a mature GAA or AGL protein to treat conditions associated with aberrant accumulation of abnormal glycogen such as occurs in Forbes-Cori Disease. The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

In certain embodiments, the disclosure provides a chimeric polypeptide comprising (i) an AGL polypeptide (e.g., an AGL polypeptide, or a functional fragment thereof) or a mature GAA polypeptide (e.g., a mature GAA polypeptide, or functional fragment thereof); and (ii) an internalizing moiety which promotes delivery to liver and/or muscle cells. AGL chimeric polypeptides of the disclosure may be used in any of the methods described herein. GAA chimeric polypeptides of the disclosure may be used in any of the methods described herein. Moreover, such AGL or GAA chimeric polypeptides may be suitable formulated and delivery via any appropriate route of administration, as described herein.

### I. AGL Polypeptides

As used herein, the AGL polypeptides include various functional fragments and variants, fusion proteins, and modified forms of the wildtype AGL polypeptide. Such functional fragments or variants, fusion proteins, and modified forms of the AGL polypeptides have at least a portion of the amino acid sequence of substantial sequence identity to the native AGL protein, and retain the function of the native AGL protein (e.g., retain the two enzymatic activities of native AGL). It should be noted that “retain the function” does not mean that the activity of a particular fragment must be identical or substantially identical to that of the native protein although, in some embodiments, it may be. However, to retain the native activity, that native activity should be at least 50%, at least 60%, at least 70%, at least 75%, at least 80%, at least 85%, at least 90%, at least 95% that of the native protein to which such activity is being compared, with the comparison being made under the same or similar conditions. In some embodiments, retaining the native activity may include scenarios in which a fragment or variant has improved activity versus the native protein to which such activity is being compared, e.g., at least 105%, at least 110%, at least 120%, or at least 125%, with the comparison being bade under the same or similar conditions.

In certain embodiments, a functional fragment, variant, or fusion protein of an AGL polypeptide comprises an amino acid sequence that is at least 80%, 85%, 90%, 95%, 97%, 98%, 99% or 100% identical to an AGL polypeptide (e.g., at least 80%, 85%, 90%, 95%, 97%, 98%, 99% or 100% identical to SEQ ID NOs: 1-3).

In certain embodiments, the AGL polypeptide for use in the chimeric polypeptides and methods of the disclosure is a full length or substantially full length AGL polypeptide. In certain embodiments, the AGL polypeptide for use in the chimeric polypeptide and methods of the disclosure is a functional fragment that has amylo-1,6-glucosidase activity and 4-alpha-glucotransferase activity.

In certain embodiments, fragments or variants of the AGL polypeptides can be obtained by screening polypeptides recombinantly produced from the corresponding fragment of the nucleic acid encoding an AGL polypeptide. In addition, fragments or variants can be chemically synthesized using techniques known in the art such as conventional Merrifield solid phase f-Moc or t-Boc chemistry. The fragments or variants can be produced (recombinantly or by chemical synthesis) and tested to identify those fragments or variants that can function as a native AGL protein, for example, by testing their ability to treat Forbes-Cori Disease in vivo and/or by confirming in vitro (e.g., in a cell free or cell based assay) that the fragment or variant has amylo-1,6-glucosidase activity and 4-alpha-glucotransferase activity. An example of an in vitro assay for testing for activity of the AGL polypeptides disclosed herein would be to treat Forbes-Cori cells with or without the AGL-containing chimeric polypeptides and then, after a period of incubation, stain the cells for the presence of glycogen, e.g., by using a periodic acid Schiff (PAS) stain.

In certain embodiments, the present disclosure contemplates modifying the structure of an AGL polypeptide for such purposes as enhancing therapeutic or prophylactic efficacy, or stability (e.g., ex vivo shelf life and resistance to proteolytic degradation in vivo). Modified polypeptides can be produced, for instance, by amino acid substitution, deletion, or addition. For instance, it is reasonable to expect, for example, that an isolated replacement of a leucine with an isoleucine or valine, an aspartate with a glutamate, a threonine with a serine, or a similar replacement of an amino acid with a structurally related amino acid (e.g., conservative mutations) will not have a major effect on the AGL biological activity of the resulting molecule. Conservative replacements are those that take place within a family of amino acids that are related in their side chains.

This disclosure further contemplates generating sets of combinatorial mutants of an AGL polypeptide, as well as truncation mutants, and is especially useful for identifying functional variant sequences. Combinatorially-derived variants can be generated which have a selective potency relative to a naturally occurring AGL polypeptide. Likewise, mutagenesis can give rise to variants which have intracellular half-lives dramatically different than the corresponding wild-type AGL polypeptide. For example, the altered protein can be rendered either more stable or less stable to proteolytic degradation or other cellular process which result in destruction of, or otherwise inactivation of AGL. Such variants can be utilized to alter the AGL polypeptide level by modulating their half-life. There are many ways by which the library of potential AGL variants sequences can be generated, for example, from a degenerate oligonucleotide sequence. Chemical synthesis of a degenerate gene sequence can be carried out in an automatic DNA synthesizer, and the synthetic genes then be ligated into an appropriate gene for expression. The purpose of a degenerate set of genes is to provide, in one mixture, all of the sequences encoding the desired set of potential polypeptide sequences. The synthesis of degenerate oligonucleotides is well known in the art (see for example, Narang, SA (1983) Tetrahedron 39:3; Itakura et al., (1981) Recombinant DNA, Proc. 3rd Cleveland Sympos. Macromolecules, ed. AG Walton, Amsterdam: Elsevier pp 273-289; Itakura et al., (1984) Annu. Rev. Biochem. 53:323; Itakura et al., (1984) Science 198:1056; Ike et al., (1983) Nucleic Acid Res. 11:477). Such techniques have been employed in the directed evolution of other proteins (see, for example, Scott et al., (1990) Science 249:386-390; Roberts et al., (1992) PNAS USA 89:2429-2433; Devlin et al., (1990) Science 249: 404-406; Cwirla et al., (1990) PNAS USA 87: 6378-6382; as well as U.S. Pat. Nos. 5,223,409, 5,198,346, and 5,096,815).

Alternatively, other forms of mutagenesis can be utilized to generate a combinatorial library. For example, AGL polypeptide variants can be generated and isolated from a library by screening using, for example, alanine scanning mutagenesis and the like (Ruf et al., (1994) Biochemistry 33:1565-1572; Wang et al., (1994) J. Biol. Chem. 269:3095-3099; Balint et al., (1993) Gene 137:109-118; Grodberg et al., (1993) Eur. J. Biochem. 218:597-601; Nagashima et al., (1993) J. Biol. Chem. 268:2888-2892; Lowman et al., (1991) Biochemistry 30:10832-10838; and Cunningham et al., (1989) Science 244:1081-1085), by linker scanning mutagenesis (Gustin et al., (1993) Virology 193:653-660; Brown et al., (1992) Mol. Cell Biol. 12:2644-2652; McKnight et al., (1982) Science 232:316); by saturation mutagenesis (Meyers et al., (1986) Science 232:613); by PCR mutagenesis (Leung et al., (1989) Method Cell Mol Biol 1:11-19); or by random mutagenesis, including chemical mutagenesis, etc. (Miller et al., (1992) A Short Course in Bacterial Genetics, CSHL Press, Cold Spring Harbor, N.Y. and Greener et al., (1994) Strategies in Mol Biol 7:32-34). Linker scanning mutagenesis, particularly in a combinatorial setting, is an attractive method for identifying truncated (bioactive) forms of the AGL polypeptide.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of the AGL polypeptides. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate sequences created by combinatorial mutagenesis techniques.

In certain embodiments, an AGL polypeptide may include a peptidomimetic. As used herein, the term “peptidomimetic” includes chemically modified peptides and peptide-like molecules that contain non-naturally occurring amino acids, peptoids, and the like. Peptidomimetics provide various advantages over a peptide, including enhanced stability when administered to a subject. Methods for identifying a peptidomimetic are well known in the art and include the screening of databases that contain libraries of potential peptidomimetics. For example, the Cambridge Structural Database contains a collection of greater than 300,000 compounds that have known crystal structures (Allen et al., Acta Crystallogr. Section B, 35:2331 (1979)). Where no crystal structure of a target molecule is available, a structure can be generated using, for example, the program CONCORD (Rusinko et al., J. Chem. Inf. Comput. Sci. 29:251 (1989)). Another database, the Available Chemicals Directory (Molecular Design Limited, Informations Systems; San Leandro Calif.), contains about 100,000 compounds that are commercially available and also can be searched to identify potential peptidomimetics of the AGL polypeptides.

In certain embodiments, an AGL polypeptide may further comprise post-translational modifications. Exemplary post-translational protein modifications include phosphorylation, acetylation, methylation, ADP-ribosylation, ubiquitination, glycosylation, carbonylation, sumoylation, biotinylation or addition of a polypeptide side chain or of a hydrophobic group. As a result, the modified AGL polypeptides may contain non-amino acid elements, such as lipids, poly- or mono-saccharides, and phosphates. Effects of such non-amino acid elements on the functionality of an AGL polypeptide may be tested for its biological activity, for example, its ability to hydrolyze glycogen or treat Forbes-Cori Disease. In certain embodiments, the AGL polypeptide may further comprise one or more polypeptide portions that enhance one or more of in vivo stability, in vivo half life, uptake/administration, and/or purification. In other embodiments, the internalizing moiety comprises an antibody or an antigen-binding fragment thereof.

In some embodiments, an AGL polypeptide is not N-glycosylated or lacks one or more of the N-glycosylation groups present in a wildtype AGL polypeptide. For example, the AGL polypeptide for use in the present disclosure may lack all N-glycosylation sites, relative to native AGL, or the AGL polypeptide for use in the present disclosure may be under-glycosylated, relative to native AGL. In some embodiments, the AGL polypeptide comprises a modified amino acid sequence that is unable to be N-glycosylated at one or more N-glycosylation sites. In some embodiments, asparagine (Asn) of at least one predicted N-glycosylation site (i.e., a consensus sequence represented by the amino acid sequence Asn-Xaa-Ser or Asn-Xaa-Thr) in the AGL polypeptide is substituted by another amino acid. Examples of Asn-Xaa-Ser sequence stretches in the AGL amino acid sequence include amino acids corresponding to amino acid positions 813-815, 839-841, 927-929, and 1032-1034 of SEQ ID NO: 1. Examples of Asn-Xaa-Thr sequence stretches in the AGL amino acid sequence include amino acids corresponding to amino acid positions 69-71, 219-221, 797-799, 1236-1238 and 1380-1382. In some embodiments, the asparagine at any one, or combination, of amino acid positions corresponding to amino acid positions 69, 219, 797, 813, 839, 927, 1032, 1236 and 1380 of SEQ ID NO: 1 is substituted or deleted. In some embodiments, the serine at any one, or combination of, amino acid positions corresponding to amino acid positions 815, 841, 929 and 1034 of SEQ ID NO: 1 is substituted or deleted. In some embodiments, the threonine at any one, or combination of, amino acid positions corresponding to amino acid positions 71, 221, 799, 1238 and 1382 of SEQ ID NO: 1 is substituted or deleted. In some embodiments, the Xaa amino acid corresponding to any one of, or combination of, amino acid positions 220, 798, 814, 840, 928, 1033, 1237 and 1381 of SEQ ID NO: 1 is deleted or replaced with a proline. The disclosure contemplates that any one or more of the foregoing examples can be combined so that an AGL polypeptide of the present disclosure lacks one or more N-glycosylation sites, and thus is either not glycosylated or is under glycosylated relative to native AGL.

In some embodiments, an AGL polypeptide is not O-glycosylated or lacks one or more of the O-glycosylation groups present in a wildtype AGL polypeptide. In some embodiments, the AGL polypeptide comprises a modified amino acid sequence that is unable to be O-glycosylated at one or more O-glycosylation sites. In some embodiments, serine or threonine at any one or more predicted O-glycosylation site in the AGL polypeptide sequence is substituted or deleted. The disclosure contemplates that any one or more of the foregoing examples can be combined so that an AGL polypeptide of the present disclosure lacks one or more N-glycosylation and/or O-glycosylation sites, and thus is either not glycosylated or is under glycosylated relative to native AGL.

In one specific embodiment of the present disclosure, an AGL polypeptide may be modified with nonproteinaceous polymers. In one specific embodiment, the polymer is polyethylene glycol (“PEG”), polypropylene glycol, or polyoxyalkylenes, in the manner as set forth in U.S. Pat. Nos. 4,640,835; 4,496,689; 4,301,144; 4,670,417; 4,791,192 or 4,179,337. PEG is a well-known, water soluble polymer that is commercially available or can be prepared by ring-opening polymerization of ethylene glycol according to methods well known in the art (Sandler and Karo, Polymer Synthesis, Academic Press, New York, Vol. 3, pages 138-161).

By the terms “biological activity”, “bioactivity” or “functional” is meant the ability of the AGL protein to carry out the functions associated with wildtype AGL proteins, for example, having oligo-1,4-1,4-glucotransferase activity and/or amylo-1,6-glucosidase activity. The terms “biological activity”, “bioactivity”, and “functional” are used interchangeably herein. As used herein, “fragments” are understood to include bioactive fragments (also referred to as functional fragments) or bioactive variants that exhibit “bioactivity” as described herein. That is, bioactive fragments or variants of AGL exhibit bioactivity that can be measured and tested. For example, bioactive fragments/functional fragments or variants exhibit the same or substantially the same bioactivity as native (i.e., wild-type, or normal) AGL protein, and such bioactivity can be assessed by the ability of the fragment or variant to, e.g., debranch glycogen via the AGL fragment's or variant's 4-alpha-glucotransferase activity and/or amylo-1,6-glucosidase activity. As used herein, “substantially the same” refers to any parameter (e.g., activity) that is at least 70% of a control against which the parameter is measured. In certain embodiments, “substantially the same” also refers to any parameter (e.g., activity) that is at least 75%, 80%, 85%, 90%, 92%, 95%, 97%, 98%, 99%, 100%, 102%. 105%, or 110% of a control against which the parameter is measured. In certain embodiments, fragments or variants of the AGL polypeptide will preferably retain at least 50%, 60%, 70%, 80%, 85%, 90%, 95% or 100% of the AGL biological activity associated with the native AGL polypeptide, when assessed under the same or substantially the same conditions.

In certain embodiments, fragments or variants of the AGL polypeptide have a half-life (t_{1/2}) which is enhanced relative to the half-life of the native protein. Preferably, the half-life of AGL fragments or variants is enhanced by at least 10%, 20%, 30%, 40%, 50%, 60%, 70%, 80%, 90%, 100%, 125%, 150%, 175%, 200%, 250%, 300%, 400% or 500%, or even by 1000% relative to the half-life of the native AGL protein. In some embodiments, the protein half-life is determined in vitro, such as in a buffered saline solution or in serum. In other embodiments, the protein half-life is an in vivo half life, such as the half-life of the protein in the serum or other bodily fluid of an animal. In addition, fragments or variants can be chemically synthesized using techniques known in the art such as conventional Merrifield solid phase f-Moc or t-Boc chemistry. The fragments or variants can be produced (recombinantly or by chemical synthesis) and tested to identify those fragments or variants that can function as well as or substantially similarly to a native AGL protein.

With respect to methods of increasing AGL bioactivity in cells, the disclosure contemplates all combinations of any of the foregoing aspects and embodiments, as well as combinations with any of the embodiments set forth in the detailed description and examples. The described methods based on administering chimeric polypeptides or contacting cells with chimeric polypeptides can be performed in vitro (e.g., in cells or culture) or in vivo (e.g., in a patient or animal model). In certain embodiments, the method is an in vitro method. In certain embodiments, the method is an in vivo method.

In some aspects, the present disclosure also provides a method of producing any of the foregoing chimeric polypeptides as described herein. Further, the present disclosure contemplates any number of combinations of the foregoing methods and compositions.

In certain aspects, an AGL polypeptide may be a fusion protein which further comprises one or more fusion domains. Well known examples of such fusion domains include, but are not limited to, polyhistidine, Glu-Glu, glutathione S transferase (GST), thioredoxin, protein A, protein G, and an immunoglobulin heavy chain constant region (Fc), maltose binding protein (MBP), which are particularly useful for isolation of the fusion proteins by affinity chromatography. For the purpose of affinity purification, relevant matrices for affinity chromatography, such as glutathione-, amylase-, and nickel- or cobalt-conjugated resins are used. Fusion domains also include “epitope tags,” which are usually short peptide sequences for which a specific antibody is available. Well known epitope tags for which specific monoclonal antibodies are readily available include FLAG, influenza virus haemagglutinin (HA), His and c-myc tags. An exemplary His tag has the sequence HHHHHH (SEQ ID NO: 23), and an exemplary c-myc tag has the sequence EQKLISEEDL (SEQ ID NO: 24). In some cases, the fusion domains have a protease cleavage site, such as for Factor Xa or Thrombin, which allows the relevant protease to partially digest the fusion proteins and thereby liberate the recombinant proteins therefrom. The liberated proteins can then be isolated from the fusion domain by subsequent chromatographic separation. In certain embodiments, the AGL polypeptides may contain one or more modifications that are capable of stabilizing the polypeptides. For example, such modifications enhance the in vitro half life of the polypeptides, enhance circulatory half life of the polypeptides or reduce proteolytic degradation of the polypeptides.

In some embodiments, an AGL protein may be a fusion protein with an Fc region of an immunoglobulin. As is known, each immunoglobulin heavy chain constant region comprises four or five domains. The domains are named sequentially as follows: CH1-hinge-CH2-CH3(-CH4). The DNA sequences of the heavy chain domains have cross-homology among the immunoglobulin classes, e.g., the CH2 domain of IgG is homologous to the CH2 domain of IgA and IgD, and to the CH3 domain of IgM and IgE. As used herein, the term, “immunoglobulin Fc region” is understood to mean the carboxyl-terminal portion of an immunoglobulin chain constant region, preferably an immunoglobulin heavy chain constant region, or a portion thereof. For example, an immunoglobulin Fc region may comprise 1) a CH1 domain, a CH2 domain, and a CH3 domain. 2) a CH1 domain and a CH2 domain, 3) a CH1 domain and a CH3 domain, 4) a CH2 domain and a CH3 domain, or 5) a combination of two or more domains and an immunoglobulin hinge region. In a preferred embodiment, the immunoglobulin Fc region comprises at least an immunoglobulin hinge region, a CH2 domain and a CH3 domain, and preferably lacks the CH1 domain. In one embodiment, the class of immunoglobulin from which the heavy chain constant region is derived is IgG (Igγ) (γ subclasses 1, 2, 3, or 4). Other classes of immunoglobulin, IgA (Igα), IgD (Igδ), IgE (Igε) and IgM (Igμ), may be used. The choice of appropriate immunoglobulin heavy chain constant regions is discussed in detail in U.S. Pat. Nos. 5,541,087, and 5,726,044. The choice of particular immunoglobulin heavy chain constant region sequences from certain immunoglobulin classes and subclasses to achieve a particular result is considered to be within the level of skill in the art. The portion of the DNA construct encoding the immunoglobulin Fc region preferably comprises at least a portion of a hinge domain, and preferably at least a portion of a CH₃ domain of Fc γ or the homologous domains in any of IgA, IgD, IgE, or IgM. Furthermore, it is contemplated that substitution or deletion of amino acids within the immunoglobulin heavy chain constant regions may be useful in the practice of the invention. One example would be to introduce amino acid substitutions in the upper CH2 region to create a Fc variant with reduced affinity for Fc receptors (Cole et al. (1997) J. IMMUNOL. 159:3613). One of ordinary skill in the art can prepare such constructs using well known molecular biology techniques.

In certain embodiments of any of the foregoing, the AGL portion of the chimeric polypeptide of the disclosure comprises an AGL polypeptide, which in certain embodiments may be a functional fragment of an AGL polypeptide or may be a substantially full length AGL polypeptide. In some embodiments, the AGL polypeptide lacks the methionine at the N-terminal-most amino acid position (i.e., lacks the methionine at the first amino acid of any one of SEQ ID NOs: 1-3). Suitable AGL polypeptides for use in the chimeric polypeptides and methods of the disclosure have oligo-1,4-1,4-glucotransferase activity and amylo-1,6-glucosidase activity, as evaluated in vitro or in vivo. Exemplary functional fragments comprise, at least 500, at least 525, at least 550, at least 575, at least 600, at least 625, at least 650, at least 675, at least 700, at least 725, at least 750, at least 775, at least 800, at least 825, at least 850, at least 875, at least 900, at least 925, at least 925, at least 950, at least 975, at least 1000, at least 1025, at least 1050, at least 1075, at least 1100, at least 1125, at least 1150, at least 1175, at least 1200, at least 1225, at least 1250, at least 1275, at least 1300, at least 1325, at least 1350, at least 1375, at least 1400, at least 1425, at least 1450, at least 1475, at least 1500, at least 1525 or at least 1532 amino consecutive amino acid residues of a full length AGL polypeptide (e.g., SEQ ID NOs: 1-3). In some embodiments, the functional fragment comprises 500-750, 500-1000, 500-1200, 500-1300, 500-1500, 1000-1100, 1000-1200, 1000-1300, 1000-1400, 1000-1500, 1000-1532 consecutive amino acids of a full-length AGL polypeptide (e.g., SEQ ID NOs: 1-3). Similarly, in certain embodiments, the disclosure contemplates chimeric proteins where the AGL portion is a variant of any of the foregoing AGL polypeptides or bioactive fragments. Exemplary variants have an amino acid sequence at least 90%, 92%, 95%, 96%, 97%, 98%, or at least 99% identical to the amino acid sequence of a native AGL polypeptide or functional fragment thereof, and such variants retain the ability to debranch glycogen via the AGL variant's oligo-1,4-1,4-glucotransferase activity and amylo-1,6-glucosidase activity. The disclosure contemplates chimeric polypeptides and the use of such polypeptides wherein the AGL portion comprises any of the AGL polypeptides, fragments, or variants described herein in combination with any internalizing moiety described herein. Moreover, in certain embodiments, the AGL portion of any of the foregoing chimeric polypeptides may, in certain embodiments, by a fusion protein. Any such chimeric polypeptides comprising any combination of AGL portions and internalizing moiety portions, and optionally including one or more linkers, one or more tags, etc., may be used in any of the methods of the disclosure.

### II. GAA Polypeptides

It has been demonstrated that mature GAA polypeptides have enhanced glycogen clearance (e.g., mature GAA is more active) as compared to the precursor mature GAA (Bijvoet, et al., 1998, Hum Mol Genet, 7(11): 1815-24), whether at low pH (e.g. lysosomal-like) or neutral pH (e.g., cytoplasmic-like) conditions. In addition, while mature GAA is a lysosomal protein that has optimal activity at lower pHs, mature GAA still retains approximately 40% activity at neutral pH (i.e., the pH of the cytoplasm) (Martin-Touaux et al., 2002, Hum Mol Genet, 11(14): 1637-45). In fact, even the reduced activity of mature GAA at neutral pH is still greater than the activity of immature GAA observed under endogenous, low pH conditions. Thus, mature GAA is suitable for use in the cytoplasm if the difficulties of delivering the protein to cytoplasm encountered in the prior art can be addressed. The present disclosure provides an approach to overcome such deficiencies and delivery mature GAA to the cytoplasm.

As used herein, the mature GAA polypeptides include variants, and in particular the mature, active forms of the protein (the active about 76 kDa or about 70 kDa forms or similar forms having an alternative starting and/or ending residue, collectively termed “mature GAA”). The term “mature GAA” refers to a polypeptide having an amino acid sequence corresponding to that portion of the immature GAA protein that, when processed endogenously, has an apparent molecular weight by SDS-PAGE of about 70 kDa to about 76 kDa, as well as similar polypeptides having alternative starting and/or ending residues, as described above. The term “mature GAA” may also refer to a GAA polypeptide lacking the signal sequence (amino acids 1-27 of SEQ ID NOs: 4 or 5). Exemplary mature GAA polypeptides include polypeptides having residues 122-782 of SEQ ID NOs: 4 or 5; residues 123-782 of SEQ ID NOs: 4 or 5; or residues 204-782 of SEQ ID NOs: 4 or 5. The term “mature GAA” includes polypeptides that are glycosylated in the same or substantially the same way as the endogenous, mature proteins, and thus have a molecular weight that is the same or similar to the predicted molecular weight. The term also includes polypeptides that are not glycosylated or are hyper-glycosylated, such that their apparent molecular weight differ despite including the same primary amino acid sequence. Any such variants or isoforms, functional fragments or variants, fusion proteins, and modified forms of the mature GAA polypeptides have at least a portion of the amino acid sequence of substantial sequence identity to the native mature GAA protein, and retain enzymatic activity. In certain embodiments, a functional fragment, variant, or fusion protein of a mature GAA polypeptide comprises an amino acid sequence that is at least 80%, 85%, 90%, 95%, 97%, 98%, 99% or 100% identical to mature GAA polypeptides set forth in one or both of SEQ ID NOs: 15 or 16, or is at least 80%. 85%. 90%, 95%. 97%. 98%, 99% or 100% identical to mature GAA polypeptides corresponding to one or more of: residues 122-782 of SEQ ID NOs: 4 or 5; residues 123-782 of SEQ ID NOs: 4 or 5; or residues 204-782 of SEQ ID NOs: 4 or 5.

In certain specific embodiments, the chimeric polypeptide comprises a mature GAA polypeptide, and does not include the 110 kDa precursor form of GAA. Thus, such a chimeric polypeptide does not have the amino-terminal sequences that directs the immature precursor form (i.e., the 110 kDa precursor form of GAA in humans) into the lysosome, and has an activity that is similar to or substantially equivalent to the activity of endogenous forms of human GAA that are about 76 kDa or about 70 kDa, with the comparison being made under the same or similar conditions (e.g. the mature GAA-chimeric polypeptide compared with the endogenous human GAA under acidic or neutral pH conditions). For example, the mature GAA may be 7-10 fold more active for glycogen hydrolysis than the 110 kDa precursor form. The mature GAA polypeptide may be the 76 kDa or the 70 kDa form of GAA, or similar forms that use alternative starting and/or ending residues. As noted in Moreland et al. (Lysosomal Acid α-Glucosidase Consists of Four Different Peptides Processsed from a Single Chain Precursor, Journal of Biological Chemistry, 280(8): 6780-6791, 2005), the nomenclature used for the processed forms of GAA is based on an apparent molecular mass as determined by SDS-PAGE. In some embodiments, mature GAA may lack the N-terminal sites that are normally glycosylated in the endoplasmic reticulum. An exemplary mature GAA polypeptide comprises SEQ ID NO: 15 or SEQ ID NO: 16. Further exemplary mature GAA polypeptide may comprise or consist of an amino acid sequence corresponding to about: residues 122-782 of SEQ ID NOs: 4 or 5; residues 123-782 of SEQ ID NOs: 4 or 5, such as shown in SEQ ID NO: 15; residues 204-782 of SEQ ID NOs: 4 or 5; residues 206-782 of SEQ ID NOs: 4 or 5; residues 288-782 of SEQ ID NOs: 4 or 5, as shown in SEQ ID NO: 16. Mature GAA polypeptides may also have the N-terminal and or C-terminal residues described above.

In other embodiments, the mature GAA polypeptides may be glycosylated, or may be not glycosylated. For those mature GAA polypeptides that are glycosylated, the glycosylation pattern may be the same as that of naturally-occurring human GAA or may be different. One or more of the glycosylation sites on the precursor mature GAA protein may be removed in the final mature GAA construct.

Mature GAA has been isolated from tissues such as bovine testes, rat liver, pig liver, human liver, rabbit muscle, human heart, human urine, and human placenta. Mature GAA may also be produced using recombinant techniques, for example by transfecting Chinese hamster ovary (CHO) cells with a vector that expresses full-length human GAA or a vector that expresses mature GAA. Recombinant human GAA (rhGAA) or mature GAA is then purified from CHO-conditioned medium, using a series of ultrafiltration, diafiltration, washing, and eluting steps, as described by Moreland et al. (Lysosomal Acid α-Glucosidase Consists of Four Different Peptides Processsed from a Single Chain Precursor, Journal of Biological Chemistry, 280(8): 6780-6791, 2005). Mature GAA fragments may be separated according to methods known in the art, such as affinity chromatography and SDS page.

In certain embodiments, mature GAA, or fragments or variants are human mature GAA.

In certain embodiments, fragments or variants of the mature GAA polypeptides can be obtained by screening polypeptides recombinantly produced from the corresponding fragment of the nucleic acid encoding a mature GAA polypeptide. In addition, fragments or variants can be chemically synthesized using techniques known in the art such as conventional Merrifield solid phase f-Moc or t-Boc chemistry. The fragments or variants can be produced (recombinantly or by chemical synthesis) and tested to identify those fragments or variants that can function as a native GAA protein, for example, by testing their ability hydrolyze glycogen and/or treat symptoms of Forbes-Cori disease.

In certain embodiments, the present disclosure contemplates modifying the structure of a mature GAA polypeptide for such purposes as enhancing therapeutic or prophylactic efficacy, or stability (e.g., ex vivo shelf life and resistance to proteolytic degradation in vivo). Such modified mature GAA polypeptides are considered functional equivalents of the naturally-occurring GAA polypeptide. Modified polypeptides can be produced, for instance, by amino acid substitution, deletion, or addition. For instance, it is reasonable to expect, for example, that an isolated replacement of a leucine with an isoleucine or valine, an aspartate with a glutamate, a threonine with a serine, or a similar replacement of an amino acid with a structurally related amino acid (e.g., conservative mutations) will not have a major effect on the GAA biological activity of the resulting molecule. Conservative replacements are those that take place within a family of amino acids that are related in their side chains.

This disclosure further contemplates generating sets of combinatorial mutants of an mature GAA polypeptide, as well as truncation mutants, and is especially useful for identifying functional variant sequences. Combinatorially-derived variants can be generated which have a selective potency relative to a naturally occurring GAA polypeptide. Likewise, mutagenesis can give rise to variants which have intracellular half-lives dramatically different than the corresponding wild-type GAA polypeptide. For example, the altered protein can be rendered either more stable or less stable to proteolytic degradation or other cellular process which result in destruction of, or otherwise inactivation of GAA function. Such variants can be utilized to alter the mature GAA polypeptide level by modulating their half-life. There are many ways by which the library of potential mature GAA variants sequences can be generated, for example, from a degenerate oligonucleotide sequence. Chemical synthesis of a degenerate gene sequence can be carried out in an automatic DNA synthesizer, and the synthetic genes then be ligated into an appropriate gene for expression. The purpose of a degenerate set of genes is to provide, in one mixture, all of the sequences encoding the desired set of potential polypeptide sequences. The synthesis of degenerate oligonucleotides is well known in the art (see for example, Narang, SA (1983) Tetrahedron 39:3; Itakura et al., (1981) Recombinant DNA, Proc. 3rd Cleveland Sympos. Macromolecules, ed. AG Walton, Amsterdam: Elsevier pp 273-289; Itakura et al., (1984) Annu. Rev. Biochem. 53:323; Itakura et al., (1984) Science 198:1056; Ike et al., (1983) Nucleic Acid Res. 11:477). Such techniques have been employed in the directed evolution of other proteins (see, for example, Scott et al., (1990) Science 249:386-390; Roberts et al., (1992) PNAS USA 89:2429-2433; Devlin et al., (1990) Science 249: 404-406; Cwirla et al., (1990) PNAS USA 87: 6378-6382; as well as U.S. Pat. Nos. 5,223,409, 5,198,346, and 5,096,815).

Alternatively, other forms of mutagenesis can be utilized to generate a combinatorial library. For example, mature GAA polypeptide variants can be generated and isolated from a library by screening using, for example, alanine scanning mutagenesis and the like (Ruf et al., (1994) Biochemistry 33:1565-1572; Wang et al., (1994) J. Biol. Chem. 269:3095-3099; Balint et al., (1993) Gene 137:109-118; Grodberg et al., (1993) Eur. J. Biochem. 218:597-601; Nagashima et al., (1993) J. Biol. Chem. 268:2888-2892; Lowmnan et al., (1991) Biochemistry 30:10832-10838; and Cunningham et al., (1989) Science 244:1081-1085), by linker scanning mutagenesis (Gustin et al., (1993) Virology 193:653-660; Brown et al., (1992) Mol. Cell Biol. 12:2644-2652; McKnight et al., (1982) Science 232:316); by saturation mutagenesis (Meyers et al., (1986) Science 232:613); by PCR mutagenesis (Leung et al., (1989) Method Cell Mol Biol 1:11-19); or by random mutagenesis, including chemical mutagenesis, etc. (Miller et al., (1992) A Short Course in Bacterial Genetics, CSHL Press, Cold Spring Harbor, N.Y.; and Greener et al., (1994) Strategies in Mol Biol 7:32-34). Linker scanning mutagenesis, particularly in a combinatorial setting, is an attractive method for identifying truncated (bioactive) forms of mature GAA.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of the mature GAA polypeptides. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate sequences created by combinatorial mutagenesis techniques.

In certain embodiments, a mature GAA polypeptide may include a peptide and a peptidomimetic. As used herein, the term “peptidomimetic” includes chemically modified peptides and peptide-like molecules that contain non-naturally occurring amino acids, peptoids, and the like. Peptidomimetics provide various advantages over a peptide, including enhanced stability when administered to a subject. Methods for identifying a peptidomimetic are well known in the art and include the screening of databases that contain libraries of potential peptidomimetics. For example, the Cambridge Structural Database contains a collection of greater than 300,000 compounds that have known crystal structures (Allen et al., Acta Crystallogr. Section B, 35:2331 (1979)). Where no crystal structure of a target molecule is available, a structure can be generated using, for example, the program CONCORD (Rusinko et al., J. Chem. Inf. Comput. Sci. 29:251 (1989)). Another database, the Available Chemicals Directory (Molecular Design Limited, Informations Systems; San Leandro Calif.), contains about 100,000 compounds that are commercially available and also can be searched to identify potential peptidomimetics of the mature GAA polypeptides.

In certain embodiments, a mature GAA polypeptide may further comprise post-translational modifications. Exemplary post-translational protein modification include phosphorylation, acetylation, methylation, ADP-ribosylation, ubiquitination, glycosylation, carbonylation, sumoylation, biotinylation or addition of a polypeptide side chain or of a hydrophobic group. As a result, the modified mature GAA polypeptides may contain non-amino acid elements, such as lipids, poly- or mono-saccharide, and phosphates. Effects of such non-amino acid elements on the functionality of a mature GAA polypeptide may be tested for its biological activity, for example, its ability to treat Forbes-Cori disease. In certain embodiments, the mature GAA polypeptide may further comprise one or more polypeptide portions that enhance one or more of in vivo stability, in vivo half life, uptake/administration, and/or purification. In other embodiments, the internalizing moiety comprises an antibody or an antigen-binding fragment thereof.

In one specific embodiment of the present disclosure, a mature GAA polypeptide may be modified with nonproteinaceous polymers. In one specific embodiment, the polymer is polyethylene glycol (“PEG”), polypropylene glycol, or polyoxyalkylenes, in the manner as set forth in U.S. Pat. Nos. 4,640,835; 4,496,689; 4,301,144; 4,670,417; 4,791,192 or 4,179,337. PEG is a well-known, water soluble polymer that is commercially available or can be prepared by ring-opening polymerization of ethylene glycol according to methods well known in the art (Sandier and Karo, Polymer Synthesis, Academic Press, New York, Vol. 3, pages 138-161).

By the terms “biological activity”, “bioactivity” or “functional” is meant the ability of the mature GAA protein to carry out the functions associated with wildtype GAA proteins, for example, the hydrolysis of α-1,4- and α-1,6-glycosidic linkages of glycogen, for example cytoplasmic glycogen. The terms “biological activity”, “bioactivity”, and “functional” are used interchangeably herein. In certain embodiments, and as described herein, a mature GAA protein or chimeric polypeptide having biological activity has the ability to hydrolyze glycogen. In other embodiments, a mature GAA protein or chimeric polypeptide having biological activity has the ability to lower the concentration of cytoplasmic and/or lysosomal glycogen. In still other embodiments, a mature GAA protein or chimeric polypeptide has the ability to treat symptoms associated with Forbes-Cori disease. As used herein, “fragments” are understood to include bioactive fragments (also referred to as functional fragments) or bioactive variants that exhibit “bioactivity” as described herein. That is, bioactive fragments or variants of mature GAA exhibit bioactivity that can be measured and tested. For example, bioactive fragments/functional fragments or variants exhibit the same or substantially the same bioactivity as native (i.e., wild-type, or normal) GAA protein, and such bioactivity can be assessed by the ability of the fragment or variant to, e.g., hydrolyze glycogen in vitro or in vivo. As used herein, “substantially the same” refers to any parameter (e.g., activity) that is at least 70% of a control against which the parameter is measured. In certain embodiments, “substantially the same” also refers to any parameter (e.g., activity) that is at least 75%, 80%, 85%, 90%, 92%, 95%, 97%, 98%, 99%, 100%, 102%, 105%, or 110% of a control against which the parameter is measured. In certain embodiments, fragments or variants of the mature GAA polypeptide will preferably retain at least 50%, 60%, 70%, 80%, 85%, 90%, 95% or 100% of the GAA biological activity associated with the native GAA polypeptide, when assessed under the same or substantially the same conditions. In certain embodiments, fragments or variants of the mature GAA polypeptide have a half-life (t_{1/2}) which is enhanced relative to the half-life of the native protein. Preferably, the half-life of mature GAA fragments or variants is enhanced by at least 10%, 20%, 30%, 40%, 50%, 60%, 70%, 80%, 90%, 100%, 125%, 150%, 175%, 200%, 250%, 300%, 400% or 500%, or even by 1000% relative to the half-life of the native GAA protein, when assessed under the same or substantially the same conditions. In some embodiments, the protein half-life is determined in vitro, such as in a buffered saline solution or in serum. In other embodiments, the protein half-life is an in vivo half life, such as the half-life of the protein in the serum or other bodily fluid of an animal. In addition, fragments or variants can be chemically synthesized using techniques known in the art such as conventional Merrifield solid phase f-Moc or t-Boc chemistry. The fragments or variants can be produced (recombinantly or by chemical synthesis) and tested to identify those fragments or variants that can function as well as or substantially similarly to a native GAA protein.

With respect to methods of increasing GAA bioactivity in cells, the disclosure contemplates all combinations of any of the foregoing aspects and embodiments, as well as combinations with any of the embodiments set forth in the detailed description and examples. The described methods based on administering chimeric polypeptides or contacting cells with chimeric polypeptides can be performed in vitro (e.g., in cells or culture) or in vivo (e.g., in a patient or animal model). In certain embodiments, the method is an in vitro method. In certain embodiments, the method is an in vivo method.

In some aspects, the present disclosure also provides a method of producing any of the foregoing chimeric polypeptides as described herein. Further, the present disclosure contemplates any number of combinations of the foregoing methods and compositions.

In certain aspects, a mature GAA polypeptide may be a fusion protein which further comprises one or more fusion domains. Well-known examples of such fusion domains include, but are not limited to, polyhistidine, Glu-Glu, glutathione S transferase (GST), thioredoxin, protein A, protein G, and an immunoglobulin heavy chain constant region (Fc), maltose binding protein (MBP), which are particularly useful for isolation of the fusion proteins by affinity chromatography. For the purpose of affinity purification, relevant matrices for affinity chromatography, such as glutathione-, amylase-, and nickel- or cobalt-conjugated resins are used. Fusion domains also include “epitope tags,” which are usually short peptide sequences for which a specific antibody is available. Well known epitope tags for which specific monoclonal antibodies are readily available include FLAG, influenza virus haemagglutinin (HA), His, and c-myc tags. An exemplary His tag has the sequence HHHHHH (SEQ ID NO: 23), and an exemplary c-myc tag has the sequence EQKLISEEDL (SEQ ID NO: 24). It is recognized that any such tags or fusions may be appended to the mature GAA portion of the chimeric polypeptide or may be appended to the internalizing moiety portion of the chimeric polypeptide, or both.

In some cases, the fusion domains have a protease cleavage site, such as for Factor Xa or Thrombin, which allows the relevant protease to partially digest the fusion proteins and thereby liberate the recombinant proteins therefrom. The liberated proteins can then be isolated from the fusion domain by subsequent chromatographic separation. In certain embodiments, the mature GAA polypeptides may contain one or more modifications that are capable of stabilizing the polypeptides. For example, such modifications enhance the in vitro half life of the polypeptides, enhance circulatory half life of the polypeptides or reducing proteolytic degradation of the polypeptides.

In some embodiments, a mature GAA polypeptide may be a fusion protein with an Fc region of an immunoglobulin. As is known, each immunoglobulin heavy chain constant region comprises four or five domains. The domains are named sequentially as follows: CH1-hinge-CH2-CH3(-CH4). The DNA sequences of the heavy chain domains have cross-homology among the immunoglobulin classes, e.g., the CH2 domain of IgG is homologous to the CH2 domain of IgA and IgD, and to the CH3 domain of IgM and IgE. As used herein, the term, “immunoglobulin Fc region” is understood to mean the carboxyl-terminal portion of an immunoglobulin chain constant region, preferably an immunoglobulin heavy chain constant region, or a portion thereof. For example, an immunoglobulin Fc region may comprise 1) a CH1 domain, a CH2 domain, and a CH3 domain, 2) a CH1 domain and a CH2 domain, 3) a CH1 domain and a CH3 domain, 4) a CH2 domain and a CH3 domain, or 5) a combination of two or more domains and an immunoglobulin hinge region. In a preferred embodiment the immunoglobulin Fe region comprises at least an immunoglobulin hinge region a CH2 domain and a CH3 domain, and preferably lacks the CH1 domain. In one embodiment, the class of immunoglobulin from which the heavy chain constant region is derived is IgG (Igγ) (γ subclasses 1, 2, 3, or 4). Other classes of immunoglobulin, IgA (Igα), IgD (Igδ), IgE (Igε) and IgM (Igμ), may be used. The choice of appropriate immunoglobulin heavy chain constant regions is discussed in detail in U.S. Pat. Nos. 5,541,087, and 5,726,044. The choice of particular immunoglobulin heavy chain constant region sequences from certain immunoglobulin classes and subclasses to achieve a particular result is considered to be within the level of skill in the art. The portion of the DNA construct encoding the immunoglobulin Fe region preferably comprises at least a portion of a hinge domain, and preferably at least a portion of a CH₃ domain of Fc γ or the homologous domains in any of IgA, IgD, IgE, or IgM. Furthermore, it is contemplated that substitution or deletion of amino acids within the immunoglobulin heavy chain constant regions may be useful in the practice of the disclosure. One example would be to introduce amino acid substitutions in the upper CH2 region to create a Fc variant with reduced affinity for Fc receptors (Cole et al. (1997) J. IMMUNOL. 159:3613). One of ordinary skill in the art can prepare such constructs using well known molecular biology techniques.

In certain embodiments of any of the foregoing, the GAA portion of the chimeric protein comprises one of the mature forms of GAA, e.g., the 76 kDa fragment, the 70 kDa fragment, similar forms that use an alternative start and/or stop site, or a functional fragment thereof. In certain embodiments, such mature GAA polypeptide or functional fragment thereof retains the ability of to hydrolyze glycogen, as evaluated in vitro or in vivo. Further, in certain embodiments, the chimeric polypeptide that comprises such a mature GAA polypeptide or functional fragment thereof can hydrolyze glycogen. Exemplary bioactive fragments comprise at least 50, at least 60, at least 75, at least 100, at least 125, at least 150, at least 175, at least 200, at least 225, at least 230, at least 250, at least 260, at least 275, or at least 300 consecutive amino acid residues of a full length mature GAA polypeptide. Similarly, in certain embodiments, the disclosure contemplates chimeric proteins where the mature GAA portion is a variant of any of the foregoing mature GAA polypeptides or functional fragments. Exemplary variants have an amino acid sequence at least 90%, 92%, 95%, 96%, 97%, 98%, or at least 99% identical to the amino acid sequence of a native GAA polypeptide or bioactive fragment thereof, and such variants retain the ability of native GAA to hydrolyze glycogen, as evaluated in vitro or in vivo. The disclosure contemplates chimeric proteins and the use of such proteins wherein the GAA portion comprises any of the mature GAA polypeptides, forms, or variants described herein in combination with any internalizing moiety described herein. Exemplary mature GAA polypeptides are set forth in SEQ ID NOs: 3 and 4. Moreover, in certain embodiments, the mature GAA portion of any of the foregoing chimeric polypeptides may, in certain embodiments, by a fusion protein. Any such chimeric polypeptides comprising any combination of GAA portions and internalizing moiety portions, and optionally including one or more linkers, one or more tags, etc., may be used in any of the methods of the disclosure.

### III. Internalizing Moieties

As used herein, the term “internalizing moiety” refers to a moiety capable of interacting with a target tissue or a cell type to effect delivery of the attached molecule into the cell (i.e., penetrate desired cell; transport across a cellular membrane; deliver across cellular membranes to, at least, the cytoplasm). Preferably, this disclosure relates to an internalizing moiety which promotes delivery to, for example, muscle cells and liver cells. Internalizing moieties having limited cross-reactivity are generally preferred. In certain embodiments, this disclosure relates to an internalizing moiety which selectively, although not necessarily exclusively, targets and penetrates muscle cells. In certain embodiments, the internalizing moiety has limited cross-reactivity, and thus preferentially targets a particular cell or tissue type. However, it should be understood that internalizing moieties of the subject disclosure do not exclusively target specific cell types. Rather, the internalizing moieties promote delivery to one or more particular cell types, preferentially over other cell types, and thus provide for delivery that is not ubiquitous. In certain embodiments, suitable internalizing moieties include, for example, antibodies, monoclonal antibodies, or derivatives or analogs thereof. Other internalizing moieties include for example, homing peptides, fusion proteins, receptors, ligands, aptamers, peptidomimetics, and any member of a specific binding pair. In certain embodiments, the internalizing moiety mediates transit across cellular membranes via an ENT2 transporter. In some embodiments, the internalizing moiety helps the chimeric polypeptide effectively and efficiently transit cellular membranes. In some embodiments, the internalizing moiety transits cellular membranes via an equilibrative nucleoside (ENT) transporter. In some embodiments, the internalizing moiety transits cellular membranes via an ENT1, ENT2, ENT3 or ENT4 transporter. In some embodiments, the internalizing moiety transits cellular membranes via an equilibrative nucleoside transporter 2 (ENT2) transporter. In some embodiments, the internalizing moiety promotes delivery into muscle cells (e.g., skeletal or cardiac muscle). In other embodiments, the internalizing moiety promotes delivery into cells other than muscle cells, e.g., neurons, epithelial cells, liver cells, kidney cells or Leydig cells. For any of the foregoing, in certain embodiments, the internalizing moiety promotes delivery of a chimeric polypeptide into the cytoplasm.

In certain embodiments, the internalizing moiety promotes delivery of a chimeric polypeptide into the cytoplasm. Without being bound by theory, regardless of whether the AGL or GAA polypeptide portion of the chimeric polypeptide comprises or consists of AGL or mature GAA, this facilitates delivery to the cytoplasm and, optionally, to the lysosome and/or autophagic vesicles.

In certain embodiments, the internalizing moiety is capable of binding polynucleotides. In certain embodiments, the internalizing moiety is capable of binding DNA. In certain embodiments, the internalizing moiety is capable of binding DNA with a K_{D} of less than 1 μM. In certain embodiments, the internalizing moiety is capable of binding DNA with a K_{D} of less than 100 nM, less than 75 nM, less than 50 nM, or even less than 30 nM. K_{D} can be measured using Surface Plasmon Resonance (SPR) or Quartz Crystal Microbalance (QCM), in accordance with currently standard methods. By way of example, an antibody or antibody fragment, including an antibody or antibody fragment comprising a VH having the amino acid sequence set forth in SEQ ID NO: 6 and a VL having an amino acid sequence set forth in SEQ ID NO: 8) is know to bind DNA with a K_{D} of less than 100 nM.

In some embodiments, the internalizing moiety targets AGL or GAA polypeptide to muscle cells and/or liver, and mediates transit of the polypeptide across the cellular membrane into the cytoplasm of the muscle cells.

As used herein, the term “internalizing moiety” refers to a moiety capable of interacting with a target tissue or a cell type. Preferably, this disclosure relates to an internalizing moiety which promotes delivery to, for example, muscle cells and liver cells. Internalizing moieties having limited cross-reactivity are generally preferred. However, it should be understood that internalizing moieties of the subject disclosure do not exclusively target specific cell types. Rather, the internalizing moieties promote delivery to one or more particular cell types, preferentially over other cell types, and thus provide for delivery that is not ubiquitous. In certain embodiments, suitable internalizing moieties include, for example, antibodies, monoclonal antibodies, or derivatives or analogs thereof; and other internalizing moieties include for example, homing peptides, fusion proteins, receptors, ligands, aptamers, peptidomimetics, and any member of a specific binding pair. In some embodiments, the internalizing moiety helps the chimeric polypeptide effectively and efficiently transit cellular membranes. In some embodiments, the internalizing moiety transits cellular membranes via an equilibrative nucleoside (ENT) transporter. In some embodiments, the internalizing moiety transits cellular membranes via an ENT1, ENT2, ENT3 or ENT4 transporter. In some embodiments, the internalizing moiety transits cellular membranes via an equilibrative nucleoside transporter 2 (ENT2) transporter. In some embodiments, the internalizing moiety promotes delivery into muscle cells (e.g., skeletal or cardiac muscle). In other embodiments, the internalizing moiety promotes delivery into cells other than muscle cells, e.g., neurons, epithelial cells, liver cells, kidney cells or Leydig cells.

(a) Antibodies

In certain aspects, an internalizing moiety may comprise an antibody, including a monoclonal antibody, a polyclonal antibody, and a humanized antibody. Without being bound by theory, such antibody may bind to an antigen of a target tissue and thus mediate the delivery of the subject chimeric polypeptide to the target tissue (e.g., muscle). In some embodiments, internalizing moieties may comprise antibody fragments, derivatives or analogs thereof, including without limitation: Fv fragments, single chain Fv (scFv) fragments, Fab′ fragments, F(ab′)2 fragments, single domain antibodies, camelized antibodies and antibody fragments, humanized antibodies and antibody fragments, human antibodies and antibody fragments, and multivalent versions of the foregoing; multivalent internalizing moieties including without limitation: Fv fragments, single chain Fv (scFv) fragments, Fab′ fragments, F(ab′)2 fragments, single domain antibodies, camelized antibodies and antibody fragments, humanized antibodies and antibody fragments, human antibodies and antibody fragments, and multivalent versions of the foregoing; multivalent internalizing moieties including without limitation: monospecific or bispecific antibodies, such as disulfide stabilized Fv fragments, scFv tandems ((scFv)₂ fragments), diabodies, tribodies or tetrabodies, which typically are covalently linked or otherwise stabilized (i.e., leucine zipper or helix stabilized) scFv fragments; receptor molecules which naturally interact with a desired target molecule. In some embodiments, the antibodies or variants thereof may be chimeric, e.g., they may include variable heavy or light regions from the murine 3E10 antibody, but may include constant regions from an antibody of another species (e.g., a human). In some embodiments, the antibodies or variants thereof may comprise a constant region that is a hybrid of several different antibody subclass constant domains (e.g., any combination of IgG1, IgG2a, IgG2b, IgG3 and IgG4).

In certain embodiments, the antibodies or variants thereof, may be modified to make them less immunogenic when administered to a subject. For example, if the subject is human, the antibody may be “humanized”; where the complementarity determining region(s) of the hybridoma-derived antibody has been transplanted into a human monoclonal antibody, for example as described in Jones, P. et al. (1986), Nature, 321, 522-525 or Tempest et al. (1991), Biotechnology, 9, 266-273. The term humanization and humanized is well understood in the art when referring to antibodies. In some embodiments, the internalizing moiety is any peptide or antibody-like protein having the complementarity determining regions (CDRs) of the 3E10 antibody sequence, or of an antibody that binds the same epitope (e.g., the same target, such as DNA) as 3E10. Also, transgenic mice, or other mammals, may be used to express humanized or human antibodies. Such humanization may be partial or complete.

In certain embodiments, the internalizing moiety comprises the monoclonal antibody 3E10 or an antigen binding fragment thereof. For example, the antibody or antigen binding fragment thereof may be monoclonal antibody 3E10, or a variant thereof that retains cell penetrating activity, or an antigen binding fragment of 3E10 or said 3E10 variant. Additionally, the antibody or antigen binding fragment thereof may be an antibody that binds to the same epitope (e.g., target, such as DNA) as 3E10, or an antibody that has substantially the same cell penetrating activity as 3E10, or an antigen binding fragment thereof. These are exemplary of agents that target ENT2. In certain embodiments, the internalizing moiety is capable of binding polynucleotides. In certain embodiments, the internalizing moiety is capable of binding DNA. In certain embodiments, the internalizing moiety is capable of binding DNA with a K_{D} of less than 1 μM. In certain embodiments, the internalizing moiety is capable of binding DNA with a K_{D} of less than 100 nM, less than 75 nM, less than 50 nM, or even less than 30 nM. K_{D} may be determined using SPR or QCM, according to manufacturer's instructions and current practice.

In certain embodiments, the antigen binding fragment is an Fv or scFv fragment thereof. Monoclonal antibody 3E10 can be produced by a hybridoma 3E10 placed permanently on deposit with the American Type Culture Collection (ATCC) under ATCC accession number PTA-2439 and is disclosed in U.S. Pat. No. 7,189,396. Additionally or alternatively, the 3E10 antibody can be produced by expressing in a host cell nucleotide sequences encoding the heavy and light chains of the 3E10 antibody. The term “3E10 antibody” or “monoclonal antibody 3E10” are used to refer to the antibody, regardless of the method used to produce the antibody. Similarly, when referring to variants or antigen-binding fragments of 3E10, such terms are used without reference to the manner in which the antibody was produced. At this point, 3E10 is generally not produced by the hybridoma but is produced recombinantly. Thus, in the context of the present application, 3E10 antibody will refer to an antibody having the sequence of the hybridoma or comprising a variable heavy chain domain comprising the amino acid sequence set forth in SEQ ID NO: 6 (which has a one amino acid substitution relative to that of the 3E10 antibody deposited with the ATCC, as described herein) and the variable light chain domain comprising the amino acid sequence set forth in SEQ ID NO: 8.

The internalizing moiety may also comprise variants of mAb 3E10, such as variants of 3E10 which retain the same cell penetration characteristics as mAb 3E10, as well as variants modified by mutation to improve the utility thereof (e.g., improved ability to target specific cell types, improved ability to penetrate the cell membrane, improved ability to localize to the cellular DNA, convenient site for conjugation, and the like). Such variants include variants wherein one or more conservative substitutions are introduced into the heavy chain, the light chain and/or the constant region(s) of the antibody. Such variants include humanized versions of 3E10 or a 3E10 variant. In some embodiments, the light chain or heavy chain may be modified at the N-terminus or C-terminus. Similarly, the foregoing description of variants applies to antigen binding fragments. Any of these antibodies, variants, or fragments may be made recombinantly by expression of the nucleotide sequence(s) in a host cell.

Monoclonal antibody 3E10 has been shown to penetrate cells to deliver proteins and nucleic acids into the cytoplasmic or nuclear spaces of target tissues (Weisbart R H et al., J Autoimmun. 1998 October; 11(5):539-46; Weisbart R H, et al. Mol Immunol. 2003 March; 39(13):783-9. Zack D J et al., J Immunol. 1996 Sep. 1; 157(5):2082-8.). Further, the VH and Vk sequences of 3E10 are highly homologous to human antibodies, with respective humanness z-scores of 0.943 and −0.880. Thus, Fv3E10 is expected to induce less of an anti-antibody response than many other approved humanized antibodies (Abhinandan K R et al., Mol. Biol. 2007 369, 852-862). A single chain Fv fragment of 3E10 possesses all the cell penetrating capabilities of the original monoclonal antibody, and proteins such as catalase, dystrophin, HSP70 and p53 retain their activity following conjugation to Fv3E10 (Hansen J E et al., Brain Res. 2006 May 9; 1088(1):187-96; Weisbart R H et al., Cancer Lett. 2003 Jun. 10; 195(2):211-9; Weisbart R H et al., J Drug Target. 2005 February; 13(2):81-7; Weisbart R H et al., J Immunol. 2000 Jun. 1; 164(11):6020-6; Hansen J E et al., J Biol Chem. 2007 Jul. 20; 282(29):20790-3). The 3E10 is built on the antibody scaffold present in all mammals; a mouse variable heavy chain and variable kappa light chain. 3E10 gains entry to cells via the ENT2 nucleotide transporter that is particularly enriched in skeletal muscle and cancer cells, and in vitro studies have shown that 3E10 is nontoxic. (Weisbart R H et al., Mol Immunol. 2003 March; 39(13):783-9; Pennycooke M et al., Biochem Biophys Res Commun. 2001 Jan. 26; 280(3):951-9).

The internalizing moiety may also include mutants of mAb 3E10, such as variants of 3E10 which retain the same or substantially the same cell penetration characteristics as mAb 3E10, as well as variants modified by mutation to improve the utility thereof (e.g., improved ability to target specific cell types, improved ability to penetrate the cell membrane, improved ability to localize to the cellular DNA, improved binding affinity, and the like). Such mutants include variants wherein one or more conservative substitutions are introduced into the heavy chain, the light chain and/or the constant region(s) of the antibody. Numerous variants of mAb 3E10 have been characterized in, e.g., U.S. Pat. No. 7,189,396 and WO 2008/091911, the teachings of which are incorporated by reference herein in their entirety.

In certain embodiments, the internalizing moiety comprises an antibody or antigen binding fragment comprising an VH domain comprising an amino acid sequence at least 80%, 85%, 90%, 95%, 96%, 97%, 99%, or 100% identical to SEQ ID NO: 6 and/or a VL domain comprising an amino acid sequence at least 85%, 90%, 95%, 96%, 97%, 99%, or 100% identical to SEQ ID NO: 8, or a humanized variant thereof. Of course, such internalizing moieties transit cells via ENT2 and/or bind the same epitope (e.g., target, such as DNA) as 3E10.

In certain embodiments, the internalizing moiety is capable of binding polynucleotides. In certain embodiments, the internalizing moiety is capable of binding DNA. In certain embodiments, the internalizing moiety is capable of binding DNA with a K_{D} Of less than 1 μM. In certain embodiments, the internalizing moiety is capable of binding DNA with a K_{D} of less than 100 nM.

In certain embodiments, the internalizing moiety is an antigen binding fragment, such as a single chain Fv of 3E10 (scFv) comprising SEQ ID NOs: 6 and 8. In certain embodiments, the internalizing moiety comprises a single chain Fv of 3E10 (or another antigen binding fragment), and the amino acid sequence of the V_{H} domain is at least 90%, 95%, 96%, 97%, 98%, 99%, or 100% identical to SEQ ID NO: 6, and amino acid sequence of the V_{L} domain is at least 90%, 95%, 96%. 97%, 98%, 99%, or 100% identical to SEQ ID NO: 8. The variant 3E10 or fragment thereof retains the function of an internalizing moiety. When the internalizing moiety is an scFv, the VH and VL domains are typically connected via a linker, such as a gly/ser linker. The VH domain may be N-terminal to the VL domain or vice versa.

In some embodiments, the internalizing moiety comprises one or more of the CDRs of the 3E10 antibody. In certain embodiments, the internalizing moiety comprises one or more of the CDRs of an antibody comprising the amino acid sequence of a V_{H} domain that is identical to SEQ ID NO: 6 and the amino acid sequence of a V_{L} domain that is identical to SEQ ID NO: 8. The CDRs of the 3E10 antibody may be determined using any of the CDR identification schemes available in the art. For example, in some embodiments, the CDRs of the 3E10 antibody are defined according to the Kabat definition as set forth in Kabat et al. Sequences of Proteins of Immunological Interest, 5th Ed. Public Health Service, National Institutes of Health, Bethesda, Md. (1991). In other embodiments, the CDRs of the 3E10 antibody are defined according to Chothia et al., 1987, J Mol Biol. 196: 901-917 and Chothia et al., 1989, Nature. 342:877-883. In other embodiments, the CDRs of the 3E10 antibody are defined according to the international ImMunoGeneTics database (IMGT) as set forth in LeFranc et al., 2003, Development and Comparative Immunology, 27: 55-77. In other embodiments, the CDRs of the 3E10 antibody are defined according to Honegger A, Pluckthun A., 2001, J Mol Biol., 309:657-670. In some embodiments, the CDRs of the 3E10 antibody are defined according to any of the CDR identification schemes discussed in Kunik et al., 2012, PLoS Comput Biol. 8(2): e1002388. In order to number residues of a 3E10 antibody for the purpose of identifying CDRs according to any of the CDR identification schemes known in the art, one may align the 3E10 antibody at regions of homology of the sequence of the antibody with a “standard” numbered sequence known in the art for the elected CDR identification scheme. Maximal alignment of framework residues frequently requires the insertion of “spacer” residues in the numbering system, to be used for the Fv region. In addition, the identity of certain individual residues at any given site number may vary from antibody chain to antibody chain due to interspecies or allelic divergence.

In certain embodiments, the internalizing moiety comprises at least 1, 2, 3, 4, or 5 of the CDRs of 3E10 as determined using the Kabat CDR identification scheme (e.g., the CDRs set forth in SEQ ID NOs: 9-14). In other embodiments, the internalizing moiety comprises at least 1, 2, 3, 4 or 5 of the CDRs of 3E10 as determined using the IMGT identification scheme (e.g., the CDRs set forth in SEQ ID NOs: 27-32). In certain embodiments, the internalizing moiety comprises all six CDRs of 3E10 as determined using the Kabat CDR identification scheme (e.g., comprises SEQ ID NOs 9-14). In other embodiments, the internalizing moiety comprises all six CDRS of 3E10 as determined using the IMGT identification scheme (e.g., which are set forth as SEQ ID NOs: 27-32). For any of the foregoing, in certain embodiments, the internalizing moiety is an antibody that binds the same epitope (e.g., the same target, such as DNA) as 3E10 and/or the internalizing moiety competes with 3E10 for binding to antigen. Exemplary internalizing moieties target and transit cells via ENT2.

The present disclosure utilizes the cell penetrating ability of 3E10 or 3E10 fragments or variants to promote delivery of AGL or mature GAA in vivo or into cells in vitro, such as into cytoplasm of cells. 3E10 and 3E10 variants and fragments are particularly well suited for this because of their demonstrated ability to effectively promote delivery to muscle cells, including skeletal and cardiac muscle, as well as diaphragm. Thus, in certain embodiments, 3E10 and 3E10 variants and fragments (or antibodies or antibody fragments that bind the same epitope and/or transit cells via ENT2) are useful for promoting effective delivery into cells in subjects, such as human patients or model organisms, having Forbes-Cori Disease or symptoms that recapitulate Forbes-Cori Disease. In certain embodiments, chimeric polypeptides in which the internalizing moiety is related to 3E10 are suitable to facilitate delivery of a polypeptide comprising AGL and/or mature GAA to the cytoplasm of cells.

As described further below, a recombinant 3E10 or 3E10-like variant or fragment can be conjugated, linked or otherwise joined to an AGL or mature GAA polypeptide. In the context of making chimeric polypeptides to AGL or a mature GAA, chemical conjugation, as well as making the chimeric polypeptide as a fusion protein is available and known in the art.

Preparation of antibodies or fragments thereof (e.g., a single chain Fv fragment encoded by V_{H}-linker-V_{L} or V_{L}-linker-V_{H} or a Fab) is well known in the art. In particular, methods of recombinant production of mAb 3E10 antibody fragments have been described in WO 2008/091911. Further, methods of generating scFv fragments of antibodies or Fabs are well known in the art. When recombinantly producing an antibody or antibody fragment, a linker may be used. For example, typical surface amino acids in flexible protein regions include Gly. Asn and Ser. One exemplary linker is provided in SEQ ID NO: 7. Permutations of amino acid sequences containing Gly, Asn and Ser would be expected to satisfy the criteria (e.g., flexible with minimal hydrophobic or charged character) for a linker sequence. Another exemplary linker is of the formula (G₄S)n, wherein n is an integer from 1-10, such as 2, 3, or 4. (SEQ ID NO: 33) Other near neutral amino acids, such as Thr and Ala, can also be used in the linker sequence.

In addition to linkers interconnecting portions of, for example, an scFv, the disclosure contemplates the use of additional linkers to, for example, interconnect the AGL or mature GAA portion to the antibody portion of the chimeric polypeptide.

Preparation of antibodies may be accomplished by any number of well-known methods for generating monoclonal antibodies. These methods typically include the step of immunization of animals, typically mice, with a desired immunogen (e.g., a desired target molecule or fragment thereof). Once the mice have been immunized, and preferably boosted one or more times with the desired immunogen(s), monoclonal antibody-producing hybridomas may be prepared and screened according to well known methods (see, for example, Kuby, Janis, Immunology, Third Edition, pp. 131-139, W.H. Freeman & Co. (1997), for a general overview of monoclonal antibody production, that portion of which is incorporated herein by reference). Over the past several decades, antibody production has become extremely robust. In vitro methods that combine antibody recognition and phage display techniques allow one to amplify and select antibodies with very specific binding capabilities. See, for example, Holt, L. J. et al., “The Use of Recombinant Antibodies in Proteomics,” Current Opinion in Biotechnology, 2000, 11:445-449, incorporated herein by reference. These methods typically are much less cumbersome than preparation of hybridomas by traditional monoclonal antibody preparation methods. In one embodiment, phage display technology may be used to generate an internalizing moiety specific for a desired target molecule. An immune response to a selected immunogen is elicited in an animal (such as a mouse, rabbit, goat or other animal) and the response is boosted to expand the immunogen-specific B-cell population. Messenger RNA is isolated from those B-cells, or optionally a monoclonal or polyclonal hybridoma population. The mRNA is reverse-transcribed by known methods using either a poly-A primer or murine immunoglobulin-specific primer(s), typically specific to sequences adjacent to the desired V_{H} and V_{L} chains, to yield cDNA. The desired V_{H} and V_{L} chains are amplified by polymerase chain reaction (PCR) typically using V_{H} and V_{L}, specific primer sets, and are ligated together, separated by a linker. V_{H} and V_{L} specific primer sets are commercially available, for instance from Stratagene, Inc. of La Jolla, Calif. Assembled V_{H}-linker-V product (encoding an scFv fragment) is selected for and amplified by PCR. Restriction sites are introduced into the ends of the V_{H}-linker-V_{L} product by PCR with primers including restriction sites and the scFv fragment is inserted into a suitable expression vector (typically a plasmid) for phage display. Other fragments, such as an Fab′ fragment, may be cloned into phage display vectors for surface expression on phage particles. The phage may be any phage, such as lambda, but typically is a filamentous phage, such as fd and M13, typically M13.

In certain embodiments, an antibody or antibody fragment is made recombinantly in a host cell. In other words, once the sequence of the antibody is known (for example, using the methods described above), the antibody can be made recombinantly using standard techniques.

In certain embodiments, the internalizing moieties may be modified to make them more resistant to cleavage by proteases. For example, the stability of an internalizing moiety comprising a polypeptide may be increased by substituting one or more of the naturally occurring amino acids in the (L) configuration with D-amino acids. In various embodiments, at least 1%, 5%, 10%, 20%, 50%, 80%, 90% or 100% of the amino acid residues of internalizing moiety may be of the D configuration. The switch from L to D amino acids neutralizes the digestion capabilities of many of the ubiquitous peptidases found in the digestive tract. Alternatively, enhanced stability of an internalizing moiety comprising an peptide bond may be achieved by the introduction of modifications of the traditional peptide linkages. For example, the introduction of a cyclic ring within the polypeptide backbone may confer enhanced stability in order to circumvent the effect of many proteolytic enzymes known to digest polypeptides in the stomach or other digestive organs and in serum. In still other embodiments, enhanced stability of an internalizing moiety may be achieved by intercalating one or more dextrorotatory amino acids (such as, dextrorotatory phenylalanine or dextrorotatory tryptophan) between the amino acids of internalizing moiety. In exemplary embodiments, such modifications increase the protease resistance of an internalizing moiety without affecting the activity or specificity of the interaction with a desired target molecule.

(b) Homing Peptides

In certain aspects, an internalizing moiety may comprise a homing peptide which selectively directs the subject chimeric AGL or mature GAA polypeptide to a target tissue (e.g., muscle). For example, delivering a chimeric polypeptide to the muscle can be mediated by a homing peptide comprising an amino acid sequence of ASSLNIA (SEQ ID NO: 34). Further exemplary homing peptides are disclosed in WO 98/53804. Homing peptides for a target tissue (or organ) can be identified using various methods well known in the art. Additional examples of homing peptides include the HIV transactivator of transcription (TAT) which comprises the nuclear localization sequence Tat48-60; Drosophila antennapedia transcription factor homeodomain (e.g., Penetratin which comprises Antp43-58 homeodomain 3rd helix); Homo-arginine peptides (e.g., Arg7 peptide-PKC-ε agonist protection of ischemic rat heart-“Arg7” disclosed as SEQ ID NO: 35) alpha-helical peptides; cationic peptides (“superpositively” charged proteins). In some embodiments, the homing peptide transits cellular membranes via an equilibrative nucleoside (ENT) transporter. In some embodiments, the homing peptide transits cellular membranes via an ENT1, ENT2, ENT3 or ENT4 transporter. In some embodiments, the homing peptide targets ENT2. In other embodiments, the homing peptide targets muscle cells. The muscle cells targeted by the homing peptide may include skeletal, cardiac or smooth muscle cells. In other embodiments, the homing peptide targets neurons, epithelial cells, liver cells, kidney cells or Leydig cells.

In certain embodiments, the homing peptide is capable of binding polynucleotides. In certain embodiments, the homing peptide is capable of binding DNA. In certain embodiments, the homing peptide is capable of binding DNA with a K_{D} of less than 1 μM. In certain embodiments, the homing peptide is capable of binding DNA with a K_{D} of less than 100 nM.

Additionally, homing peptides for a target tissue (or organ) can be identified using various methods well known in the art. Once identified, a homing peptide that is selective for a particular target tissue can be used, in certain embodiments.

An exemplary method is the in vivo phage display method. Specifically, random peptide sequences are expressed as fusion peptides with the surface proteins of phage, and this library of random peptides are infused into the systemic circulation. After infusion into host mice, target tissues or organs are harvested, the phage is then isolated and expanded, and the injection procedure repeated two more times. Each round of injection includes, by default, a negative selection component, as the injected virus has the opportunity to either randomly bind to tissues, or to specifically bind to non-target tissues. Virus sequences that specifically bind to non-target tissues will be quickly eliminated by the selection process, while the number of non-specific binding phage diminishes with each round of selection. Many laboratories have identified the homing peptides that are selective for vasculature of brain, kidney, lung, skin, pancreas, intestine, uterus, adrenal gland, retina, muscle, prostate, or tumors. See, for example, Samoylova et al., 1999, Muscle Nerve, 22:460; Pasqualini et al., 1996, Nature, 380:364; Koivunen et al., 1995, Biotechnology, 13:265; Pasqualini et al., 1995, J. Cell Biol., 130:1189; Pasqualini et al., 1996, Mole. Psych., 1:421, 423; Rajotte et al., 1998, J. Clin. Invest., 102:430; Rajotte et al., 1999, J. Biol. Chem., 274:11593. See, also, U.S. Pat. Nos. 5,622,699; 6,068,829; 6,174,687; 6,180,084; 6,232,287; 6,296,832; 6,303,573; 6,306,365. Homing peptides that target any of the above tissues may be used for targeting an AGL or GAA protein to that tissue.

(c) Additional Targeting to Lysosomes and Autophagic Vesicles

In some embodiments, the chimeric polypeptides comprise an AGL or mature GAA polypeptide, an internalizing moiety and, optionally, an additional intracellular targeting moiety. In some embodiments, the additional intracellular targeting moiety targets the chimeric polypeptide to the lysosome. In other embodiments, the additional targeting moiety targets the chimeric polypeptide to autophagic vacuoles. A traditional method of targeting a protein to lysosomes is modification of the protein with M6P residues, which directs their transport to lysosomes through interaction of M6P residues and M6PR molecules on the inner surface of structures such as the Golgi apparatus or late endosome. In certain embodiments, chimeric polypeptides of the present disclosure (e.g., polypeptides comprising mature GAA or AGL and an internalizing moiety) may further include modification, e.g., modified with the addition of one or more M6P residues, to facilitate additional targeting to the lysosome through M6PRs or in pathways independent of M6PRs. Such targeting moieties may be added, for example, at the N-terminus or C-terminus of a chimeric polypeptide, and via conjugation to 3E10 or mature GAA. In some embodiments, an M6P residue is added to the chimeric polypeptide.

In some embodiments, the chimeric polypeptides of the present disclosure are transported to autophagic vacuoles. Autophagy is a catabolic mechanism that involves cell degradation of unnecessary or dysfunctional cellular components through the lysosomal machinery. During this process, targeted cytoplasmic constituents are isolated from the rest of the cell within vesicles called autophagosomes, which are then fused with lysosomes and degraded or recycled. Uptake of proteins into autophagic vesicles is mediated by the formation of a membrane around the targeted region of a cell and subsequent fusion of the vesicle with a lysosome. Several mechanisms for autophagy are known, including macroautophagy in which organelles and proteins are sequestered within the cell in a vesicle called an autophagic vacuole. Upon fusion with the lysosome, the contents of the autophagic vacuole are degraded by acidic lysosomal hydrolases. In microautophagy, lysosomes engulf cytoplasm directly, and in chaperone-mediated autophagy, proteins with a consensus peptide sequence are bound by a hsc70-containing chaperone-cochaperone complex, which is recognized by a lysosomal protein and translocated across the lysosomal membrane. Autophagic vacuoles have a lysosomal environment (low pH), which is conducive for activity of enzymes such as mature GAA.

Autophagy naturally occurs in muscle cells of mammals (Masiero et al, 2009, Cell Metabolism, 10(6): 507-15).

In certain embodiments, the chimeric polypeptides of the present disclosure may further include modification to facilitate additional targeting to autophagic vesicles. One known chaperone-targeting motif is KFERQ-like motif (KFERQ sequence is SEQ ID NO: 36). Accordingly, this motif can be added to chimeric polypeptides as described herein, in order to target the polypeptides for autophagy. Such targeting moieties may be added, for example, at the N-terminus or C-terminus of a chimeric polypeptide, and via conjugation to 3E10 or mature GAA or AGL.

### III. Chimeric Polypeptides

Chimeric polypeptides of the present disclosure can be made in various manners.

The chimeric polypeptides may comprise any of the internalizing moieties or AGL/mature GAA polypeptides disclosed herein. In addition, any of the chimeric polypeptides disclosed herein may be utilized in any of the methods or compositions disclosed herein. In some embodiments, an internalizing moiety (e.g. an antibody or a homing peptide) is linked to any one of the AGL or mature GAA polypeptides, fragments or variants disclosed herein. In some embodiments, the chimeric polypeptide does not comprise an: i) immature GAA polypeptide of approximately 110 kDa and/or, ii) immature GAA possessing the signal sequence, i.e., amino acid residues 1-27 of SEQ ID NO: 4 or 5 and/or, iii) residues 1-56 of SEQ ID NO: 4 or 5.

In certain embodiments, the C-terminus of an AGL or mature GAA polypeptide can be linked to the N-terminus of an internalizing moiety (e.g., an antibody or a homing peptide). In some embodiments, the AGL polypeptide lacks a methionine at the N-terminal-most position (i.e., the first amino acid of any one of SEQ ID NOs: 1-3). Alternatively, the C-terminus of an internalizing moiety (e.g., an antibody or a homing peptide) can be linked to the N-terminus of an AGL or mature GAA polypeptide. In some embodiments, the AGL polypeptide lacks a methionine at the N-terminal-most position (i.e., the first amino acid of any one of SEQ ID NOs: 1-3). For example, chimeric polypeptides can be designed to place the AGL or mature GAA polypeptide at the amino or carboxy terminus of either the antibody heavy or light chain of mAb 3E10. In certain embodiments, potential configurations include the use of truncated portions of an antibody's heavy and light chain sequences (e.g., mAB 3E10) as needed to maintain the functional integrity of the attached AGL or mature GAA polypeptide. Further still, the internalizing moiety can be linked to an exposed internal (non-terminus) residue of AGL or mature GAA or a variant thereof. In further embodiments, any combination of the AGL- or mature GAA-internalizing moiety configurations can be employed, thereby resulting in an AGL:internalizing moiety ratio or mature GAA:internalizing moiety ration that is greater than 1:1 (e.g., two AGL or mature GAA molecules to one internalizing moiety).

The AGL or mature GAA polypeptide and the internalizing moiety may be linked directly to each other. Alternatively, they may be linked to each other via a linker sequence, which separates the AGL or mature GAA polypeptide and the internalizing moiety by a distance sufficient to ensure that each domain properly folds into its secondary and tertiary structures. Preferred linker sequences (1) should adopt a flexible extended conformation, (2) should not exhibit a propensity for developing an ordered secondary structure which could interact with the functional domains of the AGL or mature GAA polypeptide or the internalizing moiety, and (3) should have minimal hydrophobic or charged character, which could promote interaction with the functional protein domains. Typical surface amino acids in flexible protein regions include Gly, Asn and Ser. Permutations of amino acid sequences containing Gly, Asn and Ser would be expected to satisfy the above criteria for a linker sequence. Other near neutral amino acids, such as Thr and Ala, can also be used in the linker sequence. In a specific embodiment, a linker sequence length of about 20 amino acids can be used to provide a suitable separation of functional protein domains, although longer or shorter linker sequences may also be used. The length of the linker sequence separating the AGL or mature GAA polypeptide and the internalizing moiety can be from 5 to 500 amino acids in length, or more preferably from 5 to 100 amino acids in length. Preferably, the linker sequence is from about 5-30 amino acids in length. In preferred embodiments, the linker sequence is from about 5 to about 20 amino acids, and is advantageously from about 10 to about 20 amino acids. In other embodiments, the linker joining the AGL or mature GAA polypeptide to an internalizing moiety can be a constant domain of an antibody (e.g., constant domain of mAb 3E10 or all or a portion of an Fc region of another antibody). In certain embodiments, the linker is a cleavable linker.

In other embodiments, the AGL or mature GAA polypeptide or functional fragment thereof may be conjugated or joined directly to the internalizing moiety. For example, a recombinantly conjugated chimeric polypeptide can be produced as an in-frame fusion of the AGL or mature GAA portion and the internalizing moiety portion. In certain embodiments, the linker may be a cleavable linker. In any of the foregoing embodiments, the internalizing moiety may be conjugated (directly or via a linker) to the N-terminal or C-terminal amino acid of the AGL or mature GAA polypeptide. In other embodiments, the internalizing moiety may be conjugated (directly or indirectly) to an internal amino acid of the AGL or mature GAA polypeptide. Note that the two portions of the construct are conjugated/joined to each other. Unless otherwise specified, describing the chimeric polypeptide as a conjugation of the AGL or mature GAA portion to the internalizing moiety is used equivalently as a conjugation of the internalizing moiety to the AGL or mature GAA portion.

Regardless of whether a linker is used to interconnect the AGL or GAA portion to the internalizing moiety, the disclosure contemplates that the chimeric polypeptide may also include one or more tags (e.g., his, myc, or other tags). Such tags may be located, for example, at the N-terminus, the C-terminus, or internally. When present internally, the tag may be contiguous with a linker. Moreover, chimeric polypeptides of the disclosure may have one or more linkers.

In certain embodiments, the chimeric polypeptides comprise a “AGIH” portion (SEQ ID NO: 25) on the N-terminus of the chimeric polypeptide, and such chimeric polypeptides may be provided in the presence or absence of one or more epitope tags. In further embodiments, the chimeric polypeptide comprises a serine at the N-terminal most position of the polypeptide. In some embodiments, the chimeric polypeptides comprise an “SAGIH” (SEQ ID NO: 26) portion at the N-terminus of the polypeptide, and such chimeric polypeptides may be provided in the presence or absence of one or more epitope tags.

In certain embodiments, the chimeric polypeptides of the present disclosure can be generated using well-known cross-linking reagents and protocols. For example, there are a large number of chemical cross-linking agents that are known to those skilled in the art and useful for cross-linking the AGL or mature GAA polypeptide with an internalizing moiety (e.g., an antibody). For example, the cross-linking agents are heterobifunctional cross-linkers, which can be used to link molecules in a stepwise manner. Heterobifunctional cross-linkers provide the ability to design more specific coupling methods for conjugating proteins, thereby reducing the occurrences of unwanted side reactions such as homo-protein polymers. A wide variety of heterobifunctional cross-linkers are known in the art, including succinimidyl 4-(N-maleimidomethyl) cyclohexane-1-carboxylate (SMCC), m-Maleimidobenzoyl-N-hydroxysuccinimide ester (MBS); N-succinimidyl (4-iodoacetyl) aminobenzoate (SIAB), succinimidyl 4-(p-maleimidophenyl) butyrate (SMPB), 1-ethyl-3-(3-dimethylaminopropyl) carbodiimide hydrochloride (EDC); 4-succinimidyloxycarbonyl-a-methyl-a-(2-pyridyldithio)-tolune (SMPT), N-succinimidyl 3-(2-pyridyldithio) propionate (SPDP), succinimidyl 6-[3-(2-pyridyldithio) propionate] hexanoate (LC-SPDP). Those cross-linking agents having N-hydroxysuccinimide moieties can be obtained as the N-hydroxysulfosuccinimide analogs, which generally have greater water solubility. In addition, those cross-linking agents having disulfide bridges within the linking chain can be synthesized instead as the alkyl derivatives so as to reduce the amount of linker cleavage in vivo. In addition to the heterobifunctional cross-linkers, there exists a number of other cross-linking agents including homobifunctional and photoreactive cross-linkers. Disuccinimidyl suberate (DSS), bismaleimidohexane (BMH) and dimethylpimelimidate.2 HCl (Forbes-Cori Disease) are examples of useful homobifunctional cross-linking agents, and bis-[B-(4-azidosalicylamido)ethyl]disulfide (BASED) and N-succinimidyl-6(4′-azido-2′-nitrophenylamino)hexanoate (SANPAH) are examples of useful photoreactive cross-linkers for use in this disclosure. For a recent review of protein coupling techniques, see Means et al. (1990) Bioconjugate Chemistry. 1:2-12, incorporated by reference herein.

One particularly useful class of heterobifunctional cross-linkers, included above, contain the primary amine reactive group, N-hydroxysuccinimide (NHS), or its water soluble analog N-hydroxysulfosuccinimide (sulfo-NHS). Primary amines (lysine epsilon groups) at alkaline pH's are unprotonated and react by nucleophilic attack on NHS or sulfo-NHS esters. This reaction results in the formation of an amide bond, and release of NHS or sulfo-NHS as a by-product. Another reactive group useful as part of a heterobifunctional cross-linker is a thiol reactive group. Common thiol reactive groups include maleimides, halogens, and pyridyl disulfides. Maleimides react specifically with free sulfhydryls (cysteine residues) in minutes, under slightly acidic to neutral (pH 6.5-7.5) conditions. Halogens (iodoacetyl functions) react with —SH groups at physiological pH's. Both of these reactive groups result in the formation of stable thioether bonds. The third component of the heterobifunctional cross-linker is the spacer arm or bridge. The bridge is the structure that connects the two reactive ends. The most apparent attribute of the bridge is its effect on steric hindrance. In some instances, a longer bridge can more easily span the distance necessary to link two complex biomolecules.

In some embodiments, the chimeric polypeptide comprises multiple linkers. For example, if the chimeric polypeptide comprises an scFv internalizing moiety, the chimeric polypeptide may comprise a first linker conjugating the AGL or mature GAA to the internalizing moiety, and a second linker in the scFv conjugating the V_{H} domain (e.g., SEQ ID NO: 6) to the V_{L} domain (e.g., SEQ ID NO: 8).

Preparing protein-conjugates using heterobifunctional reagents is a two-step process involving the amine reaction and the sulfhydryl reaction. For the first step, the amine reaction, the protein chosen should contain a primary amine. This can be lysine epsilon amines or a primary alpha amine found at the N-terminus of most proteins. The protein should not contain free sulfhydryl groups. In cases where both proteins to be conjugated contain free sulfhydryl groups, one protein can be modified so that all sulfhydryls are blocked using for instance, N-ethylmaleimide (see Partis et al. (1983) J. Pro. Chem. 2:263, incorporated by reference herein). Ellman's Reagent can be used to calculate the quantity of sulfhydryls in a particular protein (see for example Ellman et al. (1958) Arch. Biochem. Biophys. 74:443 and Riddles et al. (1979) Anal. Biochem. 94:75, incorporated by reference herein).

In certain specific embodiments, chimeric polypeptides of the disclosure can be produced by using a universal carrier system. For example, an AGL or mature GAA polypeptide can be conjugated to a common carrier such as protein A, poly-L-lysine, hex-histidine, and the like. The conjugated carrier will then form a complex with an antibody which acts as an internalizing moiety. A small portion of the carrier molecule that is responsible for binding immunoglobulin could be used as the carrier.

In certain embodiments, chimeric polypeptides of the disclosure can be produced by using standard protein chemistry techniques such as those described in Bodansky, M. Principles of Peptide Synthesis, Springer Verlag, Berlin (1993) and Grant G. A. (ed.), Synthetic Peptides: A User's Guide, W. H. Freeman and Company, New York (1992). In addition, automated peptide synthesizers are commercially available (e.g., Advanced ChemTech Model 396; Milligen/Biosearch 9600). In any of the foregoing methods of cross-linking for chemical conjugation of AGL or mature GAA to an internalizing moiety, a cleavable domain or cleavable linker can be used. Cleavage will allow separation of the internalizing moiety and the AGL or mature GAA polypeptide. For example, following penetration of a cell by a chimeric polypeptide, cleavage of the cleavable linker would allow separation of AGL or mature GAA from the internalizing moiety.

In certain embodiments, the chimeric polypeptides of the present disclosure can be generated as a fusion protein containing an AGL or mature GAA polypeptide and an internalizing moiety (e.g., an antibody or a homing peptide), expressed as one contiguous polypeptide chain. In preparing such fusion protein, a fusion gene is constructed comprising nucleic acids which encode an AGL or mature GAA polypeptide and an internalizing moiety, and optionally, a peptide linker sequence to span the AGL or mature GAA polypeptide and the internalizing moiety. The use of recombinant DNA techniques to create a fusion gene, with the translational product being the desired fusion protein, is well known in the art. Both the coding sequence of a gene and its regulatory regions can be redesigned to change the functional properties of the protein product, the amount of protein made, or the cell type in which the protein is produced. The coding sequence of a gene can be extensively altered—for example, by fusing part of it to the coding sequence of a different gene to produce a novel hybrid gene that encodes a fusion protein. Examples of methods for producing fusion proteins are described in PCT applications PCT/US87/02968, PCT/US89/03587 and PCT/US90/07335, as well as Traunecker et al. (1989) Nature 339:68, incorporated by reference herein. Essentially, the joining of various DNA fragments coding for different polypeptide sequences is performed in accordance with conventional techniques, employing blunt-ended or stagger-ended termini for ligation, restriction enzyme digestion to provide for appropriate termini, filling in of cohesive ends as appropriate, alkaline phosphatase treatment to avoid undesirable joining, and enzymatic ligation. Alternatively, the fusion gene can be synthesized by conventional techniques including automated DNA synthesizers. In another method, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, Eds. Ausubel et al. John Wiley & Sons: 1992). The chimeric polypeptides encoded by the fusion gene may be recombinantly produced using various expression systems as is well known in the art (also see below).

Recombinantly conjugated chimeric polypeptides include embodiments in which the AGL polypeptide is conjugated to the N-terminus or C-terminus of the internalizing moiety.

We note that methods of making fusion proteins recombinantly are well known in the art. Any of the chimeric proteins described herein can readily be made recombinantly. This includes proteins having one or more tags and/or one or more linkers. For example, if the chimeric polypeptide comprises an scFv internalizing moiety, the chimeric polypeptide may comprise a first linker conjugating the AGL or mature GAA to the internalizing moiety, and a second linker in the scFv conjugating the V_{H} domain (e.g., SEQ ID NO: 6) to the V_{L} domain (e.g., SEQ ID NO: 8). Moreover, in certain embodiments, the chimeric polypeptides comprise a “AGIH” portion (SEQ ID NO: 25) on the N-terminus of the chimeric polypeptide, and such chimeric polypeptides may be provided in the presence or absence of one or more epitope tags. In further embodiments, the chimeric polypeptide comprises a serine at the N-terminal most position of the polypeptide. In some embodiments, the chimeric polypeptides comprise an “SAGIH” (SEQ ID NO: 26) portion at the N-terminus of the polypeptide, and such chimeric polypeptides may be provided in the presence or absence of one or more epitope tags.

In some embodiments, the immunogenicity of the chimeric polypeptide may be reduced by identifying a candidate T-cell epitope within a junction region spanning the chimeric polypeptide and changing an amino acid within the junction region as described in U.S. Patent Publication No. 2003/0166877.

Chimeric polypeptides according to the disclosure can be used for numerous purposes. We note that any of the chimeric polypeptides described herein can be used in any of the methods described herein, and such suitable combinations are specifically contemplated.

Chimeric polypeptides described herein can be used to deliver AGL or mature GAA polypeptide to cells, particular to a muscle cell, liver cell or neuron. Thus, the chimeric polypeptides can be used to facilitate transport of AGL or mature GAA to cells in vitro or in vivo. By facilitating transport to cells, the chimeric polypeptides improve delivery efficiency, thus facilitating working with AGL or mature GAA polypeptide in vitro or in vivo. Further, by increasing the efficiency of transport, the chimeric polypeptides may help decrease the amount of AGL or mature GAA needed for in vitro or in vivo experimentation.

Further detailed description of methods for making chimeric polypeptides recombinantly in cells is provided below.

The chimeric polypeptides can be used to study the function of AGL or mature GAA in cells in culture, as well as to study transport of AGL or mature GAA. The chimeric polypeptides can be used to identify substrates and/or binding partners for AGL or mature GAA in cells. The chimeric polypeptides can be used in screens to identify modifiers (e.g., small organic molecules or polypeptide modifiers) of mature GAA or AGL activity in a cell. The chimeric polypeptides can be used to help treat or alleviate the symptoms (e.g., one or more symptoms) of Forbes-Cori Disease in humans or in an animal model. The foregoing are merely exemplary of the uses for the subject chimeric polypeptides.

Any of the chimeric polypeptides described herein, including chimeric polypeptides combining any of the features of the AGL polypeptides, GAA polypeptides, internalizing moieties, and linkers, may be used in any of the methods of the disclosure.

Here and elsewhere in the specification, sequence identity refers to the percentage of residues in the candidate sequence that are identical with the residue of a corresponding sequence to which it is compared, after aligning the sequences and introducing gaps, if necessary to achieve the maximum percent identity for the entire sequence, and not considering any conservative substitutions as part of the sequence identity. Neither N- or C-terminal extensions nor insertions shall be construed as reducing identity or homology.

Methods and computer programs for the alignment of sequences and the calculation of percent identity are well known in the art and readily available. Sequence identity may be measured using sequence analysis software. For example, alignment and analysis tools available through the ExPasy bioinformatics resource portal, such as ClustalW algorithm, set to default parameters. Suitable sequence alignments and comparisons based on pairwise or global alignment can be readily selected. One example of an algorithm that is suitable for determining percent sequence identity and sequence similarity is the BLAST algorithm, which is described in Altschul et al., J Mol Biol 215:403-410 (1990). Software for performing BLAST analyses is publicly available through the National Center for Biotechnology Information (www.ncbi.nlm.nih.gov/). In certain embodiments, the now current default settings for a particular program are used for aligning sequences and calculating percent identity.

### IV. AGL/GAA-Related Nucleic Acids and Expression

In certain embodiments, the present disclosure makes use of nucleic acids for producing an AGL or mature GAA polypeptide (including functional fragments, variants, and fusions thereof). In certain specific embodiments, the nucleic acids may further comprise DNA which encodes an internalizing moiety (e.g., an antibody or a homing peptide) for making a recombinant chimeric protein of the disclosure. All these nucleic acids are collectively referred to as AGL or mature GAA nucleic acids.

The nucleic acids may be single-stranded or double-stranded, DNA or RNA molecules. In certain embodiments, the disclosure relates to isolated or recombinant nucleic acid sequences that are at least 80%, 85%, 90%, 95%, 97%, 98%, 99% or 100% identical to a region of an AGL nucleotide sequence (e.g., SEQ ID NOs: 17-22) or a mature GAA nucleotide sequence encoding a polypeptide having the amino acid sequence of either SEQ ID NO: 15 or 16. In further embodiments, the AGL or mature GAA nucleic acid sequences can be isolated, recombinant, and/or fused with a heterologous nucleotide sequence, or in a DNA library.

In certain embodiments, AGL or mature GAA nucleic acids also include nucleotide sequences that hybridize under highly stringent conditions to any of the above-mentioned native AGL or mature GAA nucleotide sequences, or complement sequences thereof. One of ordinary skill in the art will understand readily that appropriate stringency conditions which promote DNA hybridization can be varied. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the disclosure provides nucleic acids which hybridize under low stringency conditions of 6×SSC at room temperature followed by a wash at 2×SSC at room temperature.

Isolated nucleic acids which differ from the native AGL or mature GAA nucleic acids due to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this disclosure.

In certain embodiments, the recombinant AGL or mature GAA nucleic acids may be operably linked to one or more regulatory nucleotide sequences in an expression construct. Regulatory nucleotide sequences will generally be appropriate for a host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome. In a preferred embodiment, the expression vector contains a selectable marker gene to allow the selection of transformed host cells. Selectable marker genes are well known in the art and will vary with the host cell used. In certain aspects, this disclosure relates to an expression vector comprising a nucleotide sequence encoding an AGL or mature GAA polypeptide and operably linked to at least one regulatory sequence. Regulatory sequences are art-recognized and are selected to direct expression of the encoded polypeptide. Accordingly, the term regulatory sequence includes promoters, enhancers, and other expression control elements. Exemplary regulatory sequences are described in Goeddel; Gene Expression Technology: Methods in Enzymology, Academic Press, San Diego, Calif. (1990). It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

In some embodiments, a nucleic acid construct, comprising a nucleotide sequence that encodes an AGL or mature GAA polypeptide or a bioactive fragment thereof, is operably linked to a nucleotide sequence that encodes an internalizing moiety, wherein the nucleic acid construct encodes a chimeric polypeptide having AGL or mature GAA biological activity. In certain embodiments, the nucleic acid constructs may further comprise a nucleotide sequence that encodes a linker.

This disclosure also pertains to a host cell transfected with a recombinant gene which encodes an AGL or mature GAA polypeptide or a chimeric polypeptide of the disclosure. The host cell may be any prokaryotic or eukaryotic cell. For example, an AGL or mature GAA polypeptide or a chimeric polypeptide may be expressed in bacterial cells such as E. coli, insect cells (e.g., using a baculovirus expression system), yeast, or mammalian cells (e.g., CHO cells). Other suitable host cells are known to those skilled in the art.

The present disclosure further pertains to methods of producing an AGL or mature GAA polypeptide or a chimeric polypeptide of the disclosure. For example, a host cell transfected with an expression vector encoding an AGL or mature GAA polypeptide or a chimeric polypeptide can be cultured under appropriate conditions to allow expression of the polypeptide to occur. The polypeptide may be secreted and isolated from a mixture of cells and medium containing the polypeptides. Alternatively, the polypeptides may be retained cytoplasmically or in a membrane fraction and the cells harvested, lysed and the protein isolated. A cell culture includes host cells, media and other byproducts. Suitable media for cell culture are well known in the art. The polypeptides can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the polypeptides (e.g., an AGL or mature GAA polypeptide). In a preferred embodiment, the polypeptide is a fusion protein containing a domain which facilitates its purification.

A recombinant AGL or mature GAA nucleic acid can be produced by ligating the cloned gene, or a portion thereof, into a vector suitable for expression in either prokaryotic cells, eukaryotic cells (yeast, avian, insect or mammalian), or both. Expression vehicles for production of a recombinant polypeptide include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli. The preferred mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant polypeptide by the use of a baculovirus expression system. Examples of such baculovirus expression systems include pVL-derived vectors (such as pVL1392, pVL1393 and pVL941), pAcUW-derived vectors (such as pAcUWI), and pBlucBac-derived vectors (such as the β-gal containing pBlucBac III).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

The disclosure contemplates methods of producing chimeric proteins recombinantly, such as described above. Suitable vectors and host cells may be readily selected for expression of proteins in, for example, yeast or mammalian cells. Host cells may express a vector encoding a chimeric polypeptide stably or transiently. Such host cells may be cultured under suitable conditions to express chimeric polypeptide which can be readily isolated from the cell culture medium.

Chimeric polypeptides of the disclosure (e.g., polypeptides comprising an AGL or mature GAA polypeptide portion and an internalizing moiety portion) may be expressed as a single polypeptide chain or as more than one polypeptide chains. An example of a single polypeptide chain is when an AGL or GAA portion is fused inframe to an internalizing moiety, which internalizing moiety is an scFv. In certain embodiments, this single polypeptide chain is expressed from a single vector as a fusion protein.

An example of more than one polypeptide chains is when the internalizing moiety is an antibody or Fab. In certain embodiments, the heavy and light chains of the antibody or Fab may be expressed in a host cell expressing a single vector or two vectors (one expressing the heavy chain and one expressing the light chain). In either case, the AGL or GAA polypeptide may be expressed as an inframe fusion to, for example, the C-terminus of the heavy chain such that the AGL or GAA polypeptide is appended to the internalizing moiety but at a distance to the antigen binding region of the internalizing moiety.

As noted above, methods for recombinantly expressing polypeptides, including chimeric polypeptides, are well known in the art. Nucleotide sequences expressing an AGL or GAA polypeptide, such as a human AGL or GAA polypeptide, having a particular amino acid sequence are available and can be used. Moreover, nucleotide sequences expressing an internalizing moiety portion, such as expressing a 3E10 antibody, scFv, or Fab comprising the VH and VL set forth in SEQ ID NO: 6 and 8) are publicly available and can be combined with nucleotide sequence encoding suitable heavy and light chain constant regions. The disclosure contemplates nucleotide sequences encoding any of the chimeric polypeptides of the disclosure, vectors (single vector or set of vectors) comprising such nucleotide sequences, host cells comprising such vectors, and methods of culturing such host cells to express chimeric polypeptides of the disclosure.

### V. Methods of Treatment

For any of the methods described herein, the disclosure contemplates the use of any of the chimeric polypeptides described throughout the application. In addition, for any of the methods described herein, the disclosure contemplates the combination of any step or steps of one method with any step or steps from another method.

In certain embodiments, the present disclosure provides methods of delivering chimeric polypeptides to cells, including cells in culture (in vitro or ex vivo) and cells in a subject. Delivery to cells in culture, such as healthy cells or cells from a model of disease, have numerous uses. These uses include: to identify AGL and/or GAA substrates or binding partners, to evaluate localization and/or trafficking (e.g., to cytoplasm, lysosome, and/or autophagic vesicles), to evaluate enzymatic activity under a variety of conditions (e.g., pH), to assess glycogen accumulation, and the like. In certain embodiments, chimeric polypeptides of the disclosure can be used as reagents to understand AGL and/or GAA activity, localization, and trafficking in healthy or disease contexts.

Delivery to subjects, such as to cells in a subject, have numerous uses. Exemplary therapeutic uses are described below. Moreover, the chimeric polypeptides may be used for diagnostic or research purposes. For example, a chimeric polypeptide of the disclosure may be detectably labeled and administered to a subject, such as an animal model of disease or a patient, and used to image the chimeric polypeptide in the subject's tissues (e.g., localization to muscle and/or liver). Additionally exemplary uses include delivery to cells in a subject, such as to an animal model of disease (e.g., Forbes-Cori disease). By way of example, chimeric polypeptides of the disclosure may be used as reagents and delivered to animals to understand AGL and/or GAA bioactivity, localization and trafficking, protein-protein interactions, enzymatic activity, and impacts on animal physiology in healthy or diseased animals.

In certain embodiments, the present disclosure provides methods of treating conditions associated with aberrant cytoplasmic glycogen, such as Forbes-Cori Disease. These methods involve administering to the individual a therapeutically effective amount of a chimeric polypeptide as described above. These methods are particularly aimed at therapeutic and prophylactic treatments of animals, and more particularly, humans. With respect to methods for treating Forbes-Cori Disease, the disclosure contemplates all combinations of any of the foregoing aspects and embodiments, as well as combinations with any of the embodiments set forth in the detailed description and examples.

The present disclosure provides a method of delivering a chimeric polypeptide or nucleic acid construct into a cell, such as via an equilibrative nucleoside transporter (ENT) pathway, comprising contacting a cell with a chimeric polypeptide or nucleic acid construct. In some embodiments, the present disclosure provides a method of delivering a chimeric polypeptide or nucleic acid construct into a cell via an ENT1, ENT2, ENT3 or ENT4 pathway. In certain embodiments, the method comprises contacting a cell with a chimeric polypeptide, which chimeric polypeptide comprises an AGL or mature GAA polypeptide or bioactive fragment thereof and an internalizing moiety which mediates transport across a cellular membrane via an ENT2 pathway, thereby delivering the chimeric polypeptide into the cell. In certain embodiments, the cell is a muscle cell. The muscle cells targeted using the claimed method may include skeletal, cardiac or smooth muscle cells.

The present disclosure also provides a method of delivering a chimeric polypeptide or nucleic acid construct into a cell via a pathway that allows access to cells other than muscle cells. Other cell types that could be targeted using the claimed method include, for example, neurons and liver cells.

Forbes-Cori Disease, also known as Glycogen Storage Disease Type III or limit dextrinosis, is an autosomal recessive neuromuscular/hepatic disease with an estimated incidence of 1 in 83,000-100,000 live births. Forbes-Cori Disease represents approximately 24% of all Glycogen Storage Disorders. The clinical picture in Forbes-Cori Disease is reasonably well established but variable. Forbes-Cori Disease patients may suffer from skeletal myopathy, cardiomyopathy, cirrhosis of the liver, hepatomegaly, hypoglycemia, short stature, dyslipidemia, slight mental retardation, facial abnormalities, and/or increased risk of osteoporosis (Ozen et al., 2007, World J Gastroenterol, 13(18): 2545-46). Forms of Forbes-Cori Disease with muscle involvement may present muscle weakness, fatigue and muscle atrophy. Progressive muscle weakness and distal muscle wasting frequently become disabling as the patients enter the third or fourth decade of life, although this condition has been reported to begin in childhood in many Japanese patients.

The terms “treatment”, “treating”, and the like are used herein to generally mean obtaining a desired pharmacologic and/or physiologic effect. The effect may be prophylactic in terms of completely or partially preventing a disease, condition, or symptoms thereof, and/or may be therapeutic in terms of a partial or complete cure for a disease or condition and/or adverse effect attributable to the disease or condition. “Treatment” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition (e.g., arresting its development); or (c) relieving the disease or condition (e.g., causing regression of the disease or condition, providing improvement in one or more symptoms). For example, “treatment” of Forbes-Cori Disease encompasses a complete reversal or cure of the disease, or any range of improvement in conditions and/or adverse effects attributable to Forbes-Cori Disease. Merely to illustrate, “treatment” of Forbes-Cori Disease includes an improvement in any of the following effects associated with Forbes-Cori Disease or combination thereof: skeletal myopathy, cardiomyopathy, cirrhosis of the liver, hepatomegaly, hypoglycemia, short stature, dyslipidemia, failure to thrive, mental retardation, facial abnormalities, osteoporosis, muscle weakness, fatigue and muscle atrophy. Treatment may also include one or more of reduction of abnormal levels of cytoplasmic glycogen, decrease in elevated levels of one or more of alanine transaminase, aspartate transaminase, alkaline phosphatase, or creatine phosphokinase, such as decrease in such levels in serum. Improvements in any of these conditions can be readily assessed according to standard methods and techniques known in the art. Other symptoms not listed above may also be monitored in order to determine the effectiveness of treating Forbes-Cori Disease. The population of subjects treated by the method of the disease includes subjects suffering from the undesirable condition or disease, as well as subjects at risk for development of the condition or disease.

By the term “therapeutically effective dose” is meant a dose that produces the desired effect for which it is administered. The exact dose will depend on the purpose of the treatment, and will be ascertainable by one skilled in the art using known techniques (see, e.g., Lloyd (1999) The Art, Science and Technology of Pharmaceutical Compounding).

In certain embodiments, one or more chimeric polypeptides of the present disclosure can be administered, together (simultaneously) or at different times (sequentially). In addition, chimeric polypeptides of the present disclosure can be administered alone or in combination with one or more additional compounds or therapies for treating Forbes-Cori Disease or for treating glycogen storage diseases in general. For example, one or more chimeric polypeptides can be co-administered in conjunction with one or more therapeutic compounds. For example, a chimeric polypeptide comprising AGL and a chimeric polypeptide comprising GAA may both me administered to a patient. When co-administration is indicated, the combination therapy may encompass simultaneous or alternating administration. In addition, the combination may encompass acute or chronic administration. Optionally, the chimeric polypeptide of the present disclosure and additional compounds act in an additive or synergistic manner for treating Forbes-Cori Disease. Additional compounds to be used in combination therapies include, but are not limited to, small molecules, polypeptides, antibodies, antisense oligonucleotides, and siRNA molecules. Depending on the nature of the combinatory therapy, administration of the chimeric polypeptides of the disclosure may be continued while the other therapy is being administered and/or thereafter. Administration of the chimeric polypeptides may be made in a single dose, or in multiple doses. In some instances, administration of the chimeric polypeptides is commenced at least several days prior to the other therapy, while in other instances, administration is begun either immediately before or at the time of the administration of the other therapy.

In another example of combination therapy, one or more chimeric polypeptides of the disclosure can be used as part of a therapeutic regimen combined with one or more additional treatment modalities. By way of example, such other treatment modalities include, but are not limited to, dietary therapy, occupational therapy, physical therapy, ventilator supportive therapy, massage, acupuncture, acupressure, mobility aids, assistance animals, and the like. Current treatments of Forbes-Cori disease include diets high in carbohydrates and cornstarch alone or with gastric tube feedings. Patients having myopathy also are traditionally fed high-protein diets. The chimeric polypeptides of the present disclosure may be administered in conjunction with these dietary therapies. In other embodiments, the methods of the disclosure reduce the need for the patient to be on the dietary regimen.

In certain embodiments, one or more chimeric polypeptides of the present disclosure can be administered prior to or following a liver transplant

Note that although the chimeric polypeptides described herein can be used in combination with other therapies, in certain embodiments, a chimeric polypeptide is provided as the sole form of therapy. Regardless of whether administrated alone or in combination with other medications or therapeutic regiments, the dosage, frequency, route of administration, and timing of administration of the chimeric polypeptides is determined by a physician based on the condition and needs of the patient.

### VI. Gene Therapy

Conventional viral and non-viral based gene transfer methods can be used to introduce nucleic acids encoding polypeptides of AGL or mature GAA in mammalian cells or target tissues. Such methods can be used to administer nucleic acids encoding polypeptides of the disclosure (e.g., AGL or mature GAA, including variants thereof) to cells in vitro. In some embodiments, the nucleic acids encoding AGL or mature GAA are administered for in vivo or ex vivo gene therapy uses. In other embodiments, gene delivery techniques are used to study the activity of chimeric polypeptides or AGL and/or GAA polypeptide or to study Forbes-Cori disease in cell based or animal models, such as to evaluate cell trafficking, enzyme activity, and protein-protein interactions following delivery to healthy or diseased cells and tissues. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. Such methods are well known in the art.

Methods of non-viral delivery of nucleic acids encoding engineered polypeptides of the disclosure include lipofection, microinjection, biolistics, virosomes, liposomes, immunoliposomes, polycation or lipid:nucleic acid conjugates, naked DNA, artificial virions, and agent-enhanced uptake of DNA. Lipofection methods and lipofection reagents are well known in the art (e.g., Transfectam™ and Lipofectin™). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration). The preparation of lipid:nucleic acid complexes, including targeted liposomes such as immunolipid complexes, is well known to one of skill in the art.

The use of RNA or DNA viral based systems for the delivery of nucleic acids encoding AGL or mature GAA or their variants take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of polypeptides of the disclosure could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SW), human immuno deficiency virus (HIV), and combinations thereof, all of which are well known in the art.

In applications where transient expression of the polypeptides of the disclosure is preferred, adenoviral based systems are typically used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures. Construction of recombinant AAV vectors are described in a number of publications, including U.S. Pat. No. 5,173,414; Tratschin et al., Mol. Cell. Biol. 5:3251-3260 (1985); Tratschin, et al.; Mol. Cell. Biol. 4:2072-2081 (1984); Hermonat & Muzyczka, PNAS 81:6466-6470 (1984); and Samulski et al., J. Virol. 63:03822-3828 (1989).

Recombinant adeno-associated virus vectors (rAAV) are a promising alternative gene delivery systems based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system.

Replication-deficient recombinant adenoviral vectors (Ad) can be engineered such that a transgene replaces the Ad E1a, E1b, and E3 genes; subsequently the replication defector vector is propagated in human 293 cells that supply deleted gene function in trans. Ad vectors can transduce multiple types of tissues in vivo, including nondividing, differentiated cells such as those found in the liver, kidney and muscle system tissues. Conventional Ad vectors have a large carrying capacity.

Packaging cells are used to form virus particles that are capable of infecting a host cell. Such cells include 293 cells, which package adenovirus, and 42 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host, other viral sequences being replaced by an expression cassette for the protein to be expressed. The missing viral functions are supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In many gene therapy applications, it is desirable that the gene therapy vector be delivered with a high degree of specificity to a particular tissue type. A viral vector is typically modified to have specificity for a given cell type by expressing a ligand as a fusion protein with a viral coat protein on the viruses outer surface. The ligand is chosen to have affinity for a receptor known to be present on the cell type of interest. This principle can be extended to other pairs of virus expressing a ligand fusion protein and target cell expressing a receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells, such as muscle cells.

Gene therapy vectors can be delivered in vivo by administration to an individual patient, by systemic administration (e.g., intravenous, intraperitoneal, intramuscular, subdermal, or intracranial infusion) or topical application. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. For example, cells are isolated from the subject organism, transfected with a nucleic acid (gene or cDNA) encoding, e.g., AGL or mature GAA or their variants, and re-infused back into the subject organism (e.g., patient). Various cell types suitable for ex vivo transfection are well known to those of skill in the art.

In certain embodiments, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Stem cells are isolated for transduction and differentiation using known methods.

Vectors (e.g., retroviruses, adenoviruses, liposomes, etc.) containing therapeutic nucleic acids can be also administered directly to the organism for transduction of cells in vivo. Alternatively, naked DNA can be administered. Administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions of the present disclosure, as described herein.

### VII. Methods of Administration

Various delivery systems are known and can be used to administer the chimeric polypeptides of the disclosure, e.g., encapsulation in liposomes, microparticles, microcapsules, recombinant cells capable of expressing the compound, receptor-mediated endocytosis (see, e.g., Wu and Wu. 1987, J. Biol. Chem. 262:4429-4432). Methods of introduction can be enteral or parenteral, including but not limited to, intradermal, intramuscular, intraperitoneal, intravenous, subcutaneous, pulmonary, intranasal, intraocular, epidural, and oral routes. The chimeric polypeptides may be administered by any convenient mute, for example, by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the disclosure into the central nervous system by any suitable route, including epidural injection, intranasal administration or intraventricular and intrathecal injection, intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent. In certain embodiments, it may be desirable to administer the pharmaceutical compositions of the disclosure via injection or infusion into the hepatic portal vein. In certain embodiments, a hepatic vein catheter may be employed to administer the pharmaceutical compositions of the disclosure.

In certain embodiments, it may be desirable to administer the chimeric polypeptides of the disclosure locally to the area in need of treatment (e.g., muscle); this may be achieved, for example, and not by way of limitation, by local infusion during surgery, topical application, e.g., by injection, by means of a catheter, or by means of an implant, the implant being of a porous, non-porous, or gelatinous material, including membranes, such as sialastic membranes, fibers, or commercial skin substitutes.

In certain embodiments, it may be desirable to administer the chimeric polypeptides locally, for example, to the eye using ocular administration methods. In another embodiments, such local administration can be to all or a portion of the heart. For example, administration can be by intrapericardial or intramyocardial administration. Similarly, administration to cardiac tissue can be achieved using a catheter, wire, and the like intended for delivery of agents to various regions of the heart.

In other embodiments, the chimeric polypeptides of the disclosure can be delivered in a vesicle, in particular, a liposome (see Langer, 1990, Science 249:1527-1533). In yet another embodiment, the chimeric polypeptides of the disclosure can be delivered in a controlled release system. In another embodiment, a pump may be used (see Langer, 1990, supra). In another embodiment, polymeric materials can be used (see Howard et al., 1989, J. Neurosurg. 71:105). In certain specific embodiments, the chimeric polypeptides of the disclosure can be delivered intravenously.

In certain embodiments, the chimeric polypeptides are administered by intravenous infusion. In certain embodiments, the chimeric polypeptides are infused over a period of at least 10, at least 15, at least 20, or at least 30 minutes. In other embodiments, the chimeric polypeptides are infused over a period of at least 60, 90, or 120 minutes. Regardless of the infusion period, the disclosure contemplates that each infusion is part of an overall treatment plan where chimeric polypeptide is administered according to a regular schedule (e.g., weekly, monthly, etc.).

### VIII. Pharmaceutical Compositions

In certain embodiments, the subject chimeric polypeptides of the present disclosure are formulated with a pharmaceutically acceptable carrier. One or more chimeric polypeptides can be administered alone or as a component of a pharmaceutical formulation (composition). The chimeric polypeptides may be formulated for administration in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the subject chimeric polypeptides include those suitable for oral/nasal, topical, parenteral, rectal, and/or intravaginal administration. The formulations may conveniently be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient which can be combined with a carrier material to produce a single dosage form will generally be that amount of the compound which produces a therapeutic effect.

In certain embodiments, methods of preparing these formulations or compositions include combining another type of therapeutic agents and a carrier and, optionally, one or more accessory ingredients. In general, the formulations can be prepared with a liquid carrier, or a finely divided solid carrier, or both, and then, if necessary, shaping the product.

Formulations for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a subject polypeptide therapeutic agent as an active ingredient. Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

In solid dosage forms for oral administration (capsules, tablets, pills, dragees, powders, granules, and the like), one or more chimeric polypeptide therapeutic agents of the present disclosure may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like. Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

In particular, methods of the disclosure can be administered topically, either to skin or to mucosal membranes such as those on the cervix and vagina. The topical formulations may further include one or more of the wide variety of agents known to be effective as skin or stratum corneum penetration enhancers. Examples of these are 2-pyrrolidone, N-methyl-2-pyrrolidone, dimethylacetamide, dimethylformamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface active agents. Keratolytic agents such as those known in the art may also be included. Examples are salicylic acid and sulfur. Dosage forms for the topical or transdermal administration include powders, sprays, ointments, pastes, creams, lotions, gels, solutions, patches, and inhalants. The subject polypeptide therapeutic agents may be mixed under sterile conditions with a pharmaceutically acceptable carrier, and with any preservatives, buffers, or propellants which may be required. The ointments, pastes, creams and gels may contain, in addition to a subject polypeptide agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to a subject chimeric polypeptides, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

Pharmaceutical compositions suitable for parenteral administration may comprise one or more chimeric polypeptides in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

Injectable depot forms are made by forming microencapsule matrices of one or more polypeptide therapeutic agents in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

In a preferred embodiment, the chimeric polypeptides of the present disclosure are formulated in accordance with routine procedures as a pharmaceutical composition adapted for intravenous administration to human beings. Where necessary, the composition may also include a solubilizing agent and a local anesthetic such as lidocaine to ease pain at the site of the injection. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The amount of the chimeric polypeptides of the disclosure which will be effective in the treatment of a tissue-related condition or disease (e.g., Forbes-Cori Disease) can be determined by standard clinical techniques. In addition, in vitro assays may optionally be employed to help identify optimal dosage ranges. The precise dose to be employed in the formulation will also depend on the route of administration, and the seriousness of the condition, and should be decided according to the judgment of the practitioner and each subject's circumstances. However, suitable dosage ranges for intravenous administration are generally about 20-5000 micrograms of the active chimeric polypeptide per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

### IX. Animal Models

Curly-coated retriever dogs having a frame-shift mutation in their AGL gene display a disease similar to Forbes-Cori Disease in humans (Yi, et al., 2012, Disease Models and Mechanisms, 5: 804-811). These dogs possess abnormally high glycogen deposits in their liver and muscle, and, consistent with muscle and liver damage, possess high and gradually increasing levels of alaninc transaminase, aspartate transaminase, alkaline phosphatase and creatine phosphokinase in their serum. See, Yi et al. In addition these dogs displayed progressive liver fibrosis and disruption of muscle cell contractile apparatus and the fraying of myofibrils. See. Yi et al. As such, this canine model of Forbes-Cori closely resembles the human disease, with glycogen accumulation in liver and skeletal muscle that leads to progressive hepatic fibrosis and myopathy. See, Yi et al.

A mouse model of Forbes-Cori also has recently been developed. In this model, mice possess a single ENU-induced base pair mutation within the AGL gene. Similar to human patients of Forbes-Cori, these mice exhibit persistently elevated levels of alanine transaminase and aspartate transaminase, which levels are indicative of liver damage. Anstee, et al., 2011. J. Hepatology. 54(Supp 1-Abstract 887): S353. These mice also display markedly increased hepatic glycogen deposition. See, Anstee et al. As such, these mice display several key features also observed in human patients of Forbes-Cori disease.

These models provide suitable animal model systems for assessing the activity and effectiveness of the subject chimeric polypeptides. These models have correlation with symptoms of Forbes-Cori Disease, and thus provide an appropriate model for studying Forbes-Cori Disease. Activity of the polypeptide can be assessed in one or both models, and the results compared to that observed in wildtype control animals and animals not treated with the chimeric polypeptides. Assays that may be used for assessing the efficacy of any of the chimeric polypeptides disclosed herein in treating the Forbes-Cori mouse or dog include, for example: assays assessing alaninc transaminase, aspartate transaminase, alkaline phosphatase and/or creatine phosphokinase levels in the serum; assessing glycogen levels in a biopsy from the treated and untreated Forbes-Cori mice or dogs (e.g., by examining glycogen levels in a muscle or liver biopsy using, for example, periodic acid Schiff staining for determining glycogen levels); assessing tissue glycogen levels (See, e.g., Yi et al., 2012); and/or monitoring muscle function, cardiac function, liver function, and/or lifespan in the treated and untreated Forbes-Cori dogs or mice. Another example of an in vitro assay for testing activity of the chimeric polypeptides disclosed herein would be a cell or cell-free assay in which whether the ability of the chimeric polypeptides to hydrolyze 4-methylumbelliferyl-α-D-glucoside as a substrate is assessed.

Chimeric polypeptides of the disclosure have numerous uses, including in vitro and in vivo uses. In vivo uses include not only therapeutic uses but also diagnostic and research uses in, for example, any of the foregoing animal models. By way of example, chimeric polypeptides of the disclosure may be used as research reagents and delivered to animals to understand AGL and/or GAA bioactivity, localization and trafficking, protein-protein interactions, enzymatic activity, and impacts on animal physiology in healthy or diseases animals.

Chimeric polypeptides may also be used in vitro to evaluate, for example, AGL or GAA bioactivity, localization and trafficking, protein-protein interactions, and enzymatic activity in cells in culture, including healthy and AGL and/or GAA deficient cells in culture. The disclosure contemplates that chimeric polypeptides of the disclosure may be used to deliver AGL and/or GAA to cytoplasm, lysosome, and/or autophagic vesicles of cells, including cells in culture. In some embodiments, any of the chimeric polypeptides described herein may be used in cells prepared from the mutant dog or mouse, or from cells from a human afflicted with Forbes-Cori Disease, such as fibroblast cells. In addition, one skilled in the art can generate Forbes-Cori cell lines by mutating the AGL gene in a given cell line.

### X. Kits

In certain embodiments, the disclosure also provides a pharmaceutical package or kit comprising one or more containers filled with at least one chimeric polypeptide of the disclosure. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

### EXEMPLIFICATION

The disclosure now being generally described, it will be more readily understood by reference to the following examples, which are included merely for purposes of illustration of certain aspects and embodiments of the present disclosure, and are not intended to limit the disclosure. For example, the particular constructs and experimental design disclosed herein represent exemplary tools and methods for validating proper function. As such, it will be readily apparent that any of the disclosed specific constructs and experimental plan can be substituted within the scope of the present disclosure.

### Example 1: Chemical Conjugation of 3E10 and hAGL (mAb3E10*hAGL)

### Chemical Conjugation

Ten milligrams (10 mg) of 3E10 scFv comprising a light chain variable domain corresponding to SEQ ID NO: 8 (which corresponds to the light chain variable domain of the original murine 3E10 antibody deposited with the ATCC, as referenced above) interconnected by a glycine/serine linker to a heavy chain variable domain comprising the amino acid sequence of SEQ ID NO: 6 (which heavy chain variable domain has a single amino acid substitution relative to the the heavy chain variable domain of the original murine 3E10 antibody deposited with the ATCC, as referenced above) will be conjugated covalently to the 175 kDa human AGL, such as the polypeptide set forth in SEQ ID NO: 1 in the presence or absence of its N-terminal methionine, in a 1/1 molar ratio with the use of two different heterobifunctional reagents, succinimidyl 3-(2-pyridyldithio) propionate and succinimidyl trans-4-(maleimidylmethyl) cyclo-hexane-1-carboxylate. This reaction modifies the lysine residues of mAb3E10 into thiols and adds thiolreactive maleimide groups to AGL (Weisbart R H, et al., J Immunol. 2000 Jun. 1; 164(11): 6020-6). After deprotection, each modified protein will be reacted to each other to create a stable thioether bond. Chemical conjugation will be performed, and the products will be fractionated by gel filtration chromatography. The composition of the fractions will be assessed by native and SDS-PAGE in reducing and nonreducing environments. Fractions containing the greatest ratio of 3E10-AGL conjugate to free 3E10 and free AGL will be pooled and selected for use in later studies.

Other exemplary conjugates include conjugates in which the internalizing moiety is either a full length 3E10 mAb, or variant thereof, or an antigen binding fragment of the foregoing and in which the AGL, portion is an AGL isoform 1, 2 or 3 polypeptide (SEQ ID NOs: 1-3), or functional fragment of any of the foregoing. The foregoing methods can be used to make chemical conjugates that include any combination of AGL portions and internalizing moiety portions, and the foregoing are merely exemplary. Moreover, the experimental approach detailed herein can be used to test any such chimeric polypeptide

### In Vitro Assessment of Chemically Conjugated Fv3E10 and AGL

Ten to 100 uM of chemically conjugated Fv3E10-AGL, an unconjugated mixture of 3E10 and AGL, 3E10 alone, or AGL alone will be applied to semiconfluent, undifferentiated Forbes-Cori Disease or wildtype myoblasts or hepatocytes from curly-coated retrievers or humans. The specificity of 3E10-GS3-AGL for the ENT2 transporter will be validated by addition of nitrobenzylmercaptopurine riboside (NBMPR), an ENT2 specific inhibitor (Hansen et al., 2007, J. Biol. Chem., 282(29): 20790-3) to ENT2 transfected cells just prior to addition of 3E10-AGL. Eight to 24 hours later the media and cells will be collected for immunoblot and RTPCR analysis. A duplicate experiment will apply each of the above proteins onto Forbes-Cori Disease and wildtype myoblasts or hepatocytes grown on coverslips, followed by fixation and immunohistochemical detection of mAb3E10 using antibodies against mouse kappa light chain (Jackson Immunoresearch) and AGL (Pierce or Abcam).

i) Immunoblot Detection of Cell Penetrating 3E10 and AGL

Cell pellets will be resuspended in 500 ul PBS, lysed, and the supernatants will be collected for immunoblot analysis of mAb3E10 and AGL. Epitope tagging will not be employed, therefore the presence of a coincident anti-3E10 and anti-AGL immunoreactive band of ˜190 kDa (for the full length 3E10+full length AGL) in 3E10*AGL treated cells versus 3E10-alone and AGL-alone controls will constitute successful penetration of chemically conjugated 3E10*AGL. Tubulin detection will be used as a loading control.

ii) Immunofluorescence of Cell Penetrating 3E10 and AGL

Coverslips of treated cells will be washed, fixed in 100% ethanol, rehydrated, and 3E10 and AGL will be detected with anti-AGL antibodies, followed by a horseradish peroxidase conjugated secondary antibody, color development, and viewing by light microscopy.

iii) Cytopathology Analysis

Coverslips of treated cells will be washed, fixed in 100% ethanol or in 10% formalin, rehydrated, and glycogen will be detected using a periodic acid-Schiff (PAS) stain. Decreased PAS staining in the treated cells as compared to the untreated cells is indicative that the treatment is effective in reducing glycogen accumulation in the cells.

### Example 2 Genetic Construct of Fv3E10 and hAGL (Fv3E10-GS3-AGL)

Mammalian expression vectors encoding a genetic fusion of Fv3E10 and hAGL (fv3E10-GS3-hAGL, comprising the scFv of 3E10 fused to hAGL by the GS3 linker will be generated. Note that in the examples, “Fv3F10” is used to refer to an scFv of 3E10. Following transfection, the conditioned media will also be immunoblotted to detect secretion of 3E10 and hAGL into the culture media. Following concentration of the conditioned media the relative abundance of fetal and adult PCR products from Forbes-Cori Disease myoblasts (from curly-coated retrievers or humans) will be measured and compared to the appropriate controls (see Example 1) to further validate that the secreted Fv3E10-GS3-hAGL enters cells and retains the oligo-1,4-1,4-glucanotransferase activity and amylo-alpha 1,6 glucosidase activity. Note that these genetic fusions are also referred to as recombinant conjugates or recombinantly produced conjugates.

Additional recombinantly produced conjugates will similarly be made for later testing. By way of non-limiting example: (a) hAGL-GS3-3E10, (b) 3E10-GS3-hAGL, (c) hAGL-GS3-Fv3E10, (d) hAGL-3E10, (e) 3E10-hAGL, (f) hAGL-Fv3E10. Note that throughout the example, the abbreviation Fv is used to refer to a single chain Fv of 3E10. Similarly, mAb 3E10 and 3E10 are used interchangeably. These and other chimeric polypeptides can be tested using, for example, the assays detailed herein.

Create and Validate cDNA Fv3E10 Genetically Conjugated to Human AGL

i) Synthesis of the cDNA for Fv3E10

The cDNA encoding the mouse Fv3E10 variable light chain linked to the 3E10 heavy chain (SEQ ID NOs: 6 and 8) contains a mutation that enhances the cell penetrating capacity of the Fv fragment (Zack et al., 1996, J Immunol, 157(5): 2082-8). The 3E10 cDNA will be flanked by restriction sites that facilitate cloning in frame with the AGL cDNA, and synthesized and sequenced by Genscript or other qualified manufacturer of gene sequences. To maximize expression the 3E10 cDNA will be codon optimized for mammalian and pichia expression. In the event that mammals or pichia prefer a different codon for a given amino acid, the next best candidate to unify the preference will be used. The resulting cDNA will be cloned into a mammalian expression cassette and large scale preps of the plasmid pCMV-3E10-GS3-AGL will be made using the Qiagen Mega Endo-free plasmid purification kit.

ii) Transfection of Normal and Forbes-Cori Disease Cells In Vitro

Wildtype and Forbes-Cori Disease cells will be transfected with 3E10, AGL, 3E10-AGL or 3E10-GS3-AGL in a manner similar to that described above with regard to the mammalian cell transfections.

iii) Assessment of Secretion, Cell Uptake, and Glycogen Debranching Activity of 3E10-AGL

The 3E10 cDNA will possess the signal peptide of the variable kappa chain and should drive secretion of the 3E10-AGL genetic conjugate. The secretion of 3E10-AGL by transfected cells will be detected by immunoblot of conditioned media. To assess uptake of 3E10-GS3-AGL and correction of defective glycogen branching, conditioned media from the transfected cells will be applied to untransfected cells wildtype or Forbes-Cori cells. Conditioned media from pCMV (mock) transfected and pCMV-AGL transfected cells will serve as negative controls. Protein extracts from pCMV 3E10-GS3-AGL transfected cells will serve as a positive control for expression of 3E10-GS3-AGL. Twenty-four hours later total. If 3E10-GS3-AGL is secreted into the media from transfected cells, and yet does improve the defective glycogen accumulation following application to untransfected Forbes-Cori Disease myoblasts or hepatocytes, Forbes-Cori Disease myoblasts will be transfected with the ENT2 transporter cDNA (Hansen et al., 2007, J Biol Chem 282(29): 20790-3), followed two days later by addition of conditioned media. The specificity of 3E10-GS3-AGL for the ENT2 transporter will be validated by addition of nitrobenzylmercaptopurine riboside (NBMPR), an ENT2 specific inhibitor (Pennycooke et al., 2001, Biochem Biophys Res Commun. 280(3): 951-9) to ENT2 transfected cells just prior to addition of 3E10-AGL.

iv) Immunoblot Detection of Transfected 3E10-AGL and Evaluation of AGL Mediated Correction of Glycogen Branching Defects in Forbes-Cori Disease Cells

The same procedures described in Example 1 will be used.

### Production of Recombinant 3E10 Genetically Conjugated to AGL

i) Construction of Protein Expression Vectors for Pichia

Plasmid construction, transfection, colony selection and culture of Pichia will use kits and manuals per the manufacturer's instructions (Invitrogen). The cDNAs for genetically conjugated 3E10-GS3-AGL created and validated in Example 2 will be cloned into two alternative plasmids; PICZ for intracellular expression and PICZalpha for secreted expression. Protein expression form each plasmid is driven by the AOX1 promoter. Transfected pichia will be selected with Zeocin and colonies will be tested for expression of recombinant 3E10-GS3-AGL. High expressers will be selected and scaled for purification.

ii) Purification of Recombinant 3E10-GS3-AGL

cDNA fusions with mAb 3E10 Fv are ligated into the yeast expression vector pPICZA which is subsequently electroporated into the Pichia pastoris X-33 strain. Colonies are selected with Zcocin (Invitrogen, Carlsbad, Calif.) and identified with anti-his6 antibodies (Qiagen Inc, Valencia, Calif.). X-33 cells are grown in baffled shaker flasks with buffered glycerol/methanol medium, and protein synthesis is induced with 0.5% methanol according to the manufacturer's protocol (EasySelect Pichia Expression Kit, Invitrogen, Carlsbad, Calif.). The cells are lysed by two passages through a French Cell Press at 20,000 lbs/in2, and recombinant protein is purified from cell pellets solubilized in 9M guanidine HCl and 2% NP40 by immobilized metal ion affinity chromatography (IMAC) on Ni-NTAAgarose (Qiagen, Valencia, Calif.). Bound protein is eluted in 50 mM NaH2PO4 containing 300 mM NaCl, 500 mM imidazole, and 25% glycerol. Samples of eluted fractions are electrophoresed in 4-20% gradient SDSPAGE (NuSep Ltd, Frenchs Forest. Australia), and recombinant proteins is identified by Western blotting to nitrocellulose membranes developed with cargo-specific mouse antibodies followed by alkalinephosphatase-conjugated goat antibodies to mouse IgG. Alkaline phosphatase activity is measured by the chromogenic substrate, nitroblue tetrazolium chloride/5-bromo-4-chloro-3-indolylphosphate p-toluidine salt. Proteins are identified in SDS-PAGE gels with GelCode Blue Stain Reagent (Pierce Chemical Co., Rockford, Ill.). Eluted protein is concentrated, reconstituted with fetal calf serum to 5%, and exchange dialyzed 100-fold in 30,000 MWCO spin filters (Millipore Corp., Billerica, Mass.) against McCoy's medium (Mediatech, Inc., Hemdon, Va.) containing 5% glycerol.

iii) Quality Assessment and Formulation

Immunoblot against 3E10 and AGL will be used to verify the size and identity of recombinant proteins, followed by silver staining to identify the relative purity of among preparations of 3E10, AGL and 3E10-GS3-AGL. Recombinant material will be formulated in a buffer and concentration (˜0.5 mg/ml) that is consistent with the needs of subsequent in vivo administrations.

iv) In Vitro Assessment of Recombinant Material

The amount of 3E10-GS3-AGL in the conditioned media that alleviates the glycogen debranching defects in Forbes-Cori Disease cells will be determined using the methods described above. This value will be used as a standard to extrapolate the amount of pichia-derived recombinant 3E10-GS3-AGL needed to alleviate the glycogen debranching defects. The relative oligo-1,4-1,4-glucanotransferase activity and amylo-alpha 1,6 glucosidase activity of mammalian cell-derived and pichia-derived recombinant 3E10-GS3-AGL on Forbes-Cori Disease and wildtype myoblasts or hepatocytes will be assessed.

### Example 3 In Vivo Assessment of Muscle Targeted AGL in Forbes-Cori Disease Curly-Coated Retrievers

### Selection of a Forbes-Cori Disease1 Dog Model for Evaluation

The Forbes-Cori Disease Curly-Coated Retriever (“the Forbes-Cori dog”) recapitulates human Forbes-Cori Disease in many ways (Yi et al. 2012). These dogs do not make functional AGL protein (Yi et al., 2012). To control whether a superphysiological level of AGL is a beneficial treatment or detrimental, 3E10-AGL (such as Fv3E10-AGL; either as a recombinant fusion protein or a chemical conjugate, and in the presence or absence of linker) will be administered to Forbes-Cori dogs.

### Selection of Dose of AGL

There currently is no information regarding the stability, clearance rate, volume of distribution or half-life of the injected material in the Forbes-Cori dogs, and doses applied to cell lines in vitro do not faithfully extrapolate to animals. Therefore, the evaluation dose of 3E10 chemically or genetically conjugated to AGL delivered to the Forbes-Cori dogs must be determined empirically. To minimize the confounding effect of a neutralizing immune response to 3E10-GS3-AGL and to maximize the ability to demonstrate a therapeutic effect, two high doses of 5 mg/kg of 3E10-GS3-AGL delivered in one week, followed by assessment of changes in disease endpoints, will be assessed. The development of anti-3E10-AGL antibodies will also be monitored. If it is established that intravenous 3E10*AGL or 3E10-GS3-AGL results in an improvement in glycogen branching defects or aberrant glycogen storage, subsequent in vivo assessments in other models (e.g., primates) will be initiated, followed by assessment of changes in glycogen debranching defects, as determined by immunohistochemistry (e.g., PAS staining). A positive evaluation of 3E10*AGL or 3E10-GS3-AGL will justify the production of quantities of GLP-grade material needed to perform a more thorough pharmacology and toxicology assessment, and thus determine a dose and dosing range for pre-IND studies.

### Materials and Methods

i) Injection of Chemically and Genetically Conjugated 3E10-AGL

3E10*AGL or 3E10-GS3-AGL will be formulated and diluted in a buffer that is consistent with intravenous injection (e.g. sterile saline solution or a buffered solution of 50 mM Tris-HCl, pH 7.4, 0.15 M NaCl). The amount of 3E10*AGL or 3E10-GS3-AGL given to each dog will be calculated as follows: dose (mg/kg)×dog weight (kg)×stock concentration (mg/ml)=volume (ml) of stock per dog, q.s. to 100 ul with vehicle.

ii) Blood Collection

Blood will be collected by cardiac puncture at the time that animals are sacrificed for tissue dissection. Serum will be removed and frozen at −80° C. To minimize the effects of thawing and handling all analysis of 3E10*AGL or 3E10-GS3-AGL circulating in the blood will be performed on the same day.

iii) Tissue Collection and Preparation

Sampled tissues will be divided for immunoblot, glycogen analysis, formalin-fixed paraffin-embedded tissue blocks and frozen sections in OCT. Heart, liver, lung, spleen, kidneys, quadriceps, EDL, soleus, diaphragm, and biceps tissue (50-100 mg) will be subdivided and frozen in plastic tubes for further processing for immunoblot and glycogen analysis. Additional samples of heart, liver, lung, spleen, kidneys, quadriceps, EDL, soleus, diaphragm, and biceps will be subdivided, frozen in OCT tissue sectioning medium, or fixed in 3% glutaraldehyde formaldehyde fixation for 24 to 48 hours at 4° C. and embedded in Epon resin, or fixed in 10% NBF and processed into paraffin blocks.

iv) Histological Evaluation

Epon-resin embedded samples will be cut at 1 m and stained with PAS-Richardson's stain for glycogen staining. Reduced levels of glycogen accumulation in tissues (e.g., muscle or liver) of Forbes-Cori dogs treated with 3E10*AGL or 3E10-GS3-AGL as compared to control treated Forbes-Cori dogs is indicative that the 3E10*AGL or 3E10-GS3-AGL is capable of reducing glycogen levels in vivo.

The paraffin-embedded samples will be cut at 1 μm and stained with H&E or trichrome stains. Reduced fibrosis in liver samples or reduced fraying of myofibrils in muscle samples from Forbes-Cori dogs treated with 3E10*AGL or 3E10-GS3-AGL as compared to control treated Forbes-Cori dogs is indicative that the 3E10*AGL or 3E10-GS3-AGL is capable of reducing a liver and/or muscular defect in these dogs.

v) Immunofluorescence

Exogenously delivered AGL will be detected using a polyclonal or monoclonal anti-AGL antibody, such as the antibody used in Chen et al., Am J Hum Genet. 1987 December; 41(6): 1002-15 or Parker et al. (2007). AMP-activated protein kinase does not associate with glycogen alpha-particles from rat liver. Biochem. Biophys. Res. Commun. 362:811-815. Ten micrometer frozen sections will be cut and placed on Superfrost Plus microscope slides.

vi) Immunoblot

Immunoblot will be used to detect 3E10 and AGL immune reactive material in 3E10-AGL treated muscles and hepatic tissues. Protein isolation and immunoblot detection of 3E10 and AGL will be performed according to routine immunoblot methods. AGL will be detected with an antibody specific for this protein. Antibody detection of blotted proteins will use NBT/BCIP as a substrate. Controls will include vehicle and treated Forbes-Cori dogs and vehicle and treated homozygous wildtype dogs.

vii) Analysis of Circulating 3E10-AGL

An ELISA specific to human 3E10-AGL will be developed and validated using available anti-human AGL antibodies and horseradish peroxidase conjugated anti-mouse secondary antibody (Jackson Immunoresearch). Recombinant 3E10-AGL will be diluted and used to generate a standard curve. Levels of 3E10-AGL will be determined from dilutions of

serum (normalized to ng/ml of serum) or tissue extracts (normalized to ng/mg of tissue). Controls will include vehicle and treated Forbes-Cori and wildtype dogs.

viii) Monitoring of Anti-3E10-AGL Antibody Responses

Purified 3E10-AGL used to inject Forbes-Cori dogs will be plated onto high-binding 96 well ELISA plates at 1 ug/ml in coating buffer (Pierce Biotech), allowed to coat overnight, blocked for 30 minutes in 1% nonfat drymilk (Biorad) in TBS, and rinsed three times in TBS. Two-fold dilutions of sera from vehicle and 3E10-AGL injected animals will be loaded into wells, allowed to incubate for 30 minutes at 37° C., washed three times, incubated with horseradish peroxidase (HRP)-conjugated rabbit anti-dog IgA, IgG, and IgM, allowed to incubate for 30 minutes at 37° C., and washed three times. Dog anti-3E10-AGL antibodies will be detected with TMB liquid substrate and read at 405 nm in ELISA plate reader. A polyclonal rabbit anti-dog AGL antibody, followed by HRP-conjugated goat anti-rabbit will serve as the positive control antibody reaction. Any absorbance at 405 nm greater than that of vehicle treated Forbes-Cori dogs will constitute a positive anti-3E10-AGL antibody response. Controls will include vehicle and treated wildtype dogs and Forbes-Cori dogs.

ix) Assessing Serum Enzyme Levels

Blood is collected from saphenous or jugular veins for each dog every one to three weeks for the duration of the study. Samples are tested for levels of alanine transaminase, aspartate transaminase, alkaline phosphatase, and/or creatine phosphokinase. Decrease in the elevated levels of one or more of these enzymes is indicative of reduction of some of the pathological effects of cytoplasmic glycogen accumulation.

x) Tissue Glycogen Analysis

Tissue glycogen content is assayed enzymatically using the protocol described in Yi et al. (2012). Frozen liver or muscle tissues (50-100 mg) are homogenized in ice-cold de-ionized water (20 ml water/g tissue) and sonicated three times for 20 seconds with 30-second intervals between pulses using an ultrasonicator. Homogenates are clarified by centrifugation at 12,000 g for 20 minutes at 4° C. Supernatant (20 ul) is mixed with 55 ul of water, boiled for 3 minutes and cooled to room temperature. Amyloglucosidase (Sigma) solution (25 ul diluted 1:50 into 0.1M potassium acetate buffer, pH 5.5) is added and the reaction incubated at 37° C. for 90 minutes. Samples are boiled for 3 minutes to stop the reaction and centrifuged at top speed for 3 minutes in a bench-top microcentrifuge. Supernatant (30 ul) is mixed with 1 ml of Infinity Glucose reagent (Thermo Scientific) and left at room temperature for at least 10 minutes. Absorbance at 340 nm is measured using a UV-1700 spectrophotometer. A reaction without amyloglucosidase is used for background correction for each sample. A standard curve is generated using standard glucose solutions in the reaction with Infinity Glucose reagent (0-120 uM final glucose concentration in the reaction).

xi) Survival Assessment

Those treated and untreated diseased and control dogs that are not sacrificed in the experiments described above will be monitored in a survival study. Specifically, the disease state, treatment conditions and date of death of the animals will be recorded. A survival curve will be prepared based on the results of this study.

xii) Statistical Analysis

Pairwise comparisons will employ Student's t-test. Comparisons among multiple groups will employ ANOVA. In both cases a p-value <0.05 will be considered statistically significant.

The foregoing experimental scheme will similarly be used to evaluate other chimeric polypeptides. By way of non-limiting example, this scheme will be used to evaluate chemical conjugates and fusion proteins having an AGL portion (or a fragment thereof) and an internalizing moiety portion.

### Example 4: Chemical Conjugation of 3E10 and hGAA (mAh3E10*hGAA)

### Chemical Conjugation

Ten milligrams (10 mg) of 3E10 scFv comprising a light chain variable domain corresponding to SEQ ID NO: 8 interconnected by a glycine/serine linker to a heavy chain variable domain comprising the amino acid sequence of SEQ ID NO: 6 will be conjugated covalently to the 70-76 kDa human mature GAA in a 1/1 molar ratio with the use of two different heterobifunctional reagents, succinimidyl 3-(2-pyridyldithio) propionate and succinimidyl trans-4-(maleimidylmethyl) cyclo-hexane-1-carboxylate. This reaction modifies the lysine residues of mAb3E10 into thiols and adds thiolreactive maleimide groups to GAA (Weisbart R H, et al., J Immunol. 2000 Jun. 1; 164(11): 6020-6). After deprotection, each modified protein will be reacted to each other to create a stable thioether bond. Chemical conjugation will be performed, and the products will be fractionated by gel filtration chromatography. The composition of the fractions will be assessed by native and SDS-PAGE in reducing and nonreducing environments. Fractions containing the greatest ratio of 3E10-GAA conjugate to free 3E10 and free GAA will be pooled and selected for use in later studies.

The foregoing methods can be used to make chemical conjugates that include any combination of GAA portions and internalizing moiety portions, and the foregoing are merely exemplary. Moreover, the experimental approach detailed herein can be used to test any such chimeric polypeptide

### In Vitro Assessment of Chemically Conjugated 3E0 and GAA

Ten to 100 uM of chemically conjugated 3E10-GAA, an unconjugated mixture of mAb 3E10 and GAA, mAb 3E10 alone, or mature GAA alone will be applied to seniconfluent, undifferentiated Forbes-Cori Disease or wildtype myoblasts or hepatocytes from curly-coated retrievers or humans. The specificity of 3E10-GS3-GAA for the ENT2 transporter will be validated by addition of nitrobenzylmercaptopurine riboside (NBMPR), an ENT2 specific inhibitor (Hansen et al., 2007, J. Biol. Chem., 282(29): 20790-3) to ENT2 transfected cells just prior to addition of 3E10-GAA. Eight to 24 hours later the media and cells will be collected for immunoblot and RTPCR analysis. A duplicate experiment will apply each of the above proteins onto Forbes-Cori Disease and wildtype myoblasts or hepatocytes grown on coverslips, followed by fixation and immunohistochemical detection of mAb3E10 using antibodies against mouse kappa light chain (Jackson Immunorescarch) and GAA (Pierce or Abcam).

i) Immunoblot Detection of Cell Penetrating 3E10 and GAA

Cell pellets will be resuspended in 500 ul PBS, lysed, and the supernatants will be collected for immunoblot analysis of mAb3E10 and GAA. Epitope tagging will not be employed, therefore the presence of a coincident anti-3E10 and anti-GAA immunoreactive band of ˜190 kDa (for the full length 3E10+mature GAA) in 3E10*GAA treated cells versus 3E10-alone and GAA-alone controls will constitute successful penetration of chemically conjugated 3E10*GAA. Tubulin detection will be used as a loading control.

ii) Immunofluorescence of Cell Penetrating 3E10 and GAA

Coverslips of treated cells will be washed, fixed in 100% ethanol, rehydrated, and 3E10 and GAA will be detected with anti-GAA antibodies, followed by a horseradish peroxidase conjugated secondary antibody, color development, and viewing by light microscopy.

iii) Cytopathology Analysis

Coverslips of treated cells will be washed, fixed in 100% ethanol or in 10% formalin, rehydrated, and glycogen will be detected using a periodic acid-Schiff (PAS) stain. Decreased PAS staining in the treated cells as compared to the untreated cells is indicative that the treatment is effective in reducing glycogen accumulation in the cells.

### Example 5 Genetic Construct of Fv3E10 and hGAA (Fv3E10-GS3-GAA)

Mammalian expression vectors encoding a genetic fusion of Fv3E10 and hGAA (fv3E10-GS3-hGAA, comprising the scFv of mAb 3E10 fused to hGAA by the GS3 linker will be generated. Note that in the examples, “Fv3E10” is used to refer to an scFv of 3E10. Following transfection, the conditioned media will also be immunoblotted to detect secretion of 3E10 and hGAA into the culture media. Following concentration of the conditioned media the relative abundance of fetal and adult PCR products from Forbes-Cori Disease myoblasts (from curly-coated retrievers or humans) will be measured and compared to the appropriate controls (see Example 1) to further validate that the secreted Fv3E10-GS3-hGAA enters cells and retains the glucosidase activity. Note that these genetic fusions are also referred to as recombinant conjugates or recombinantly produced conjugates.

Additional recombinantly produced conjugates will similarly be made for later testing. By way of non-limiting example: (a) hGAA-GS3-3E10, (b) 3E10-GS3-hGAA, (c) hGAA-GS3-Fv3E10, (d) hGAA-3E10, (e) 3E10-hGAA, (f) hGAA-Fv3E10. Note that throughout the example, the abbreviation Fv is used to refer to a single chain Fv of 3E10. Similarly, mAb 3E10 and 3E10 are used interchangeably. These and other chimeric polypeptides can be tested using, for example, the assays detailed herein.

Create and Validate cDNA Fv3E10 Genetically Conjugated to Human GAA

i) Synthesis of the cDNA for Fv3E10

The cDNA encoding the mouse Fv3E10 variable light chain linked to the 3E10 heavy chain (SEQ ID NOs: 6 and 8) contains a mutation that enhances the cell penetrating capacity of the Fv fragment (Zack et al., 1996, J Immunol, 157(5): 2082-8). The 3E10 cDNA will be flanked by restriction sites that facilitate cloning in frame with the GAA cDNA, and synthesized and sequenced by Genscript or other qualified manufacturer of gene sequences. To maximize expression the 3E10 cDNA will be codon optimized for mammalian and pichia expression. In the event that mammals or pichia prefer a different codon for a given amino acid, the next best candidate to unify the preference will be used. The resulting cDNA will be cloned into a mammalian expression cassette and large scale preps of the plasmid pCMV-3E10-GS3-GAA will be made using the Qiagen Mega Endo-free plasmid purification kit.

ii) Transfection of Normal and Forbes-Cori Disease Cells In Vitro

Wildtype and Forbes-Cori Disease cells will be transfected with 3E10, GAA, 3E10-GAA or 3E10-GS3-GAA in a manner similar to that described above with regard to the mammalian cell transfections.

iii) Assessment of Secretion, Cell Uptake, and Glycogen Hydrolysis Activity of 3E10-GAA

The 3E10 cDNA will possess the signal peptide of the variable kappa chain and should drive secretion of the 3E10-GAA genetic conjugate. The secretion of 3E10-GAA by transfected cells will be detected by immunoblot of conditioned media. To assess uptake of 3E10-GS3-GAA and correction of defective glycogen branching, conditioned media from the transfected cells will be applied to untransfected cells wildtype or Forbes-Cori cells. Conditioned media from pCMV (mock) transfected and pCMV-GAA transfected cells will serve as negative controls. Protein extracts from pCMV 3E10-GS3-GAA transfected cells will serve as a positive control for expression of 3E10-GS3-GAA. Twenty-four hours later total. If 3E10-GS3-GAA is secreted into the media from transfected cells, and yet does improve the defective glycogen accumulation following application to untransfected Forbes-Cori Disease myoblasts or hepatocytes, Forbes-Cori Disease myoblasts will be transfected with the ENT2 transporter cDNA (Hansen et al., 2007, J Biol Chem 282(29): 20790-3), followed two days later by addition of conditioned media. The specificity of 3E10-GS3-GAA for the ENT2 transporter will be validated by addition of nitrobenzylmercaptopurine riboside (NBMPR), an ENT2 specific inhibitor (Pennycooke et al., 2001, Biochem Biophys Res Commun. 280(3): 951-9) to ENT2 transfected cells just prior to addition of 3E10-GAA.

iv) Immunoblot Detection of Transfected 3E10-GAA and Evaluation of GAA Mediated Correction of Glycogen Branching Defects in Forbes-Cori Disease Cells

The same procedures described in Example 1 will be used.

### Production of Recombinant 3E10 Genetically Conjugated to GAA

i) Construction of Protein Expression Vectors for Pichia

Plasmid construction, transfection, colony selection and culture of Pichia will use kits and manuals per the manufacturer's instructions (Invitrogen). The cDNAs for genetically conjugated 3E10-GS3-GAA created and validated in Example 2 will be cloned into two alternative plasmids; PICZ for intracellular expression and PICZalpha for secreted expression. Protein expression form each plasmid is driven by the AOX1 promoter. Transfected pichia will be selected with Zeocin and colonies will be tested for expression of recombinant 3E10-GS3-GAA. High expressers will be selected and scaled for purification.

ii) Purification of Recombinant 3E10-GS3-GAA

cDNA fusions with mAb 3E10 Fv are ligated into the yeast expression vector pPICZA which is subsequently electroporated into the Pichia pastoris X-33 strain. Colonies are selected with Zeocin (Invitrogen, Carlsbad, Calif.) and identified with anti-his6 antibodies (Qiagen Inc, Valencia, Calif.). X-33 cells are grown in baffled shaker flasks with buffered glycerol/methanol medium, and protein synthesis is induced with 0.5% methanol according to the manufacturer's protocol (EasySelect Pichia Expression Kit, Invitrogen, Carlsbad, Calif.). The cells are lysed by two passages through a French Cell Press at 20,000 lbs/in2, and recombinant protein is purified from cell pellets solubilized in 9M guanidine HCl and 2% NP40 by immobilized metal ion affinity chromatography (IMAC) on Ni-NTAAgarose (Qiagen, Valencia, Calif.). Bound protein is eluted in 50 mM NaH2PO4 containing 300 mM NaCl, 500 mM imidazole, and 25% glycerol. Samples of eluted fractions are electrophoresed in 4-20% gradient SDSPAGE (NuSep Ltd, Frenchs Forest, Australia), and recombinant proteins is identified by Western blotting to nitrocellulose membranes developed with cargo-specific mouse antibodies followed by alkalinephosphatase-conjugated goat antibodies to mouse IgG. Alkaline phosphatase activity is measured by the chromogenic substrate, nitroblue tetrazolium chloride/5-bromo-4-chloro-3-indolylphosphate p-toluidine salt. Proteins are identified in SDS-PAGE gels with GelCode Blue Stain Reagent (Pierce Chemical Co., Rockford, Ill.). Eluted protein is concentrated, reconstituted with fetal calf serum to 5%, and exchange dialyzed 100-fold in 30,000 MWCO spin filters (Millipore Corp., Billerica, Mass.) against McCoy's medium (Mediatech, Inc., Herndon, Va.) containing 5% glycerol.

iii) Quality Assessment and Formulation

Immunoblot against 3E10 and GAA will be used to verify the size and identity of recombinant proteins, followed by silver staining to identify the relative purity of among preparations of 3E10, GAA and 3E10-GS3-GAA. Recombinant material will be formulated in a buffer and concentration (˜0.5 mg/ml) that is consistent with the needs of subsequent in vivo administrations.

iv) In Vitro Assessment of Recombinant Material

The amount of 3E10-GS3-GAA in the conditioned media that alleviates the glycogen debranching defects in Forbes-Cori Disease cells will be determined using the methods described above. This value will be used as a standard to extrapolate the amount of pichia-derived recombinant 3E10-GS3-GAA needed to alleviate the glycogen debranching defects. The relative glycogen hydrolysis activity of mammalian cell-derived and pichia-derived recombinant 3E10-GS3-GAA on Forbes-Cori Disease and wildtype myoblasts or hepatocytes will be assessed.

### Example 6 In Vivo Assessment of Muscle Targeted GAA in Forbes-Cori Disease Curly-Coated Retrievers

### Selection of a Forbes-Cori Disease1 Dog Model for Evaluation

The Forbes-Cori Disease Curly-Coated Retriever recapitulates human Forbes-Cori Disease in many ways (Yi et al. 2012). These dogs do not make functional GAA protein (Yi et al., 2012). To control whether a superphysiological level of GAA is a beneficial treatment or detrimental, 3E10-GAA will be administered to Forbes-Cori Disease dogs.

### Selection of Dose of GAA

There currently is no information regarding the stability, clearance rate, volume of distribution or half-life of the injected material in the Forbes-Cori dogs, and doses applied to cell lines in vitro do not faithfully extrapolate to animals. Therefore, the evaluation dose of 3E10 chemically or genetically conjugated to GAA delivered to the Forbes-Cori dogs must be determined empirically. To minimize the confounding effect of a neutralizing immune response to 3E10-GS3-GAA and to maximize the ability to demonstrate a therapeutic effect, two high doses of 5 mg/kg of 3E10-GS3-GAA delivered in one week, followed by assessment of changes in disease endpoints, will be assessed. The development of anti-3E10-GAA antibodies will also be monitored. If it is established that intravenous 3E10*GAA or 3E10-GS3-GAA results in an improvement in glycogen branching defects or aberrant glycogen storage, subsequent in vivo assessments in other models (e.g., primates) will be initiated, followed by assessment of changes in glycogen debranching defects, as determined by immunohistochemistry (e.g., PAS staining). A positive evaluation of 3E10*GAA or 3E10-GS3-GAA will justify the production of quantities of GLP-grade material needed to perform a more thorough pharmacology and toxicology assessment, and thus determine a dose and dosing range for pre-IND studies.

### Materials and Methods

i) Injection of Chemically and Genetically Conjugated 3E10-GAA

3E10*GAA or 3E10-GS3-GAA will be formulated and diluted in a buffer that is consistent with intravenous injection (e.g. sterile saline solution or a buffered solution of 50 mM Tris-HCl, pH 7.4, 0.15 M NaCl). The amount of 3E10*GAA or 3E10-GS3-GAA given to each dog will be calculated as follows: dose (mg/kg)×dog weight (kg)×stock concentration (mg/ml)=volume (ml) of stock per dog, q.s. to 100 ul with vehicle.

ii) Blood Collection

Blood will be collected by cardiac puncture at the time that animals are sacrificed for tissue dissection. Serum will be removed and frozen at −80° C. To minimize the effects of thawing and handling all analysis of 3E10*GAA or 3E10-GS3-GAA circulating in the blood will be performed on the same day.

iii) Tissue Collection and Preparation

Sampled tissues will be divided for immunoblot, glycogen analysis, formalin-fixed paraffin-embedded tissue blocks and frozen sections in OCT. Heart, liver, lung, spleen, kidneys, quadriceps, EDL, solcus, diaphragm, and biceps tissue (50-100 mg) will be subdivided and frozen in plastic tubes for further processing for immunoblot and glycogen analysis. Additional samples of heart, liver, lung, spleen, kidneys, quadriceps, EDL, soleus, diaphragm, and biceps will be subdivided, frozen in OCT tissue sectioning medium, or fixed in 3% glutaraldehyde formaldehyde fixation for 24 to 48 hours at 4° C. and embedded in Epon resin, or fixed in 10% NBF and processed into paraffin blocks.

iv) Histological Evaluation

Epon-resin embedded samples will be cut at 1 μm and stained with PAS-Richardson's stain for glycogen staining. Reduced levels of glycogen accumulation in tissues (e.g., muscle or liver) of Forbes-Cori dogs treated with 3E10*GAA or 3E10-GS3-GAA as compared to control treated Forbes-Cori dogs is indicative that the 3E10*GAA or 3E10-GS3-GAA is capable of reducing glycogen levels in vivo.

The paraffin-embedded samples will be cut at 1 μm and stained with H&E or trichrome stains. Reduced fibrosis in liver samples or reduced fraying of myofibrils in muscle samples from Forbes-Cori dogs treated with 3E10*GAA or 3E10-GS3-GAA as compared to control treated Forbes-Cori dogs is indicative that the 3E10*GAA or 3E10-GS3-GAA is capable of reducing a liver and/or muscular defect in these dogs.

v) Immunofluorescence

Exogenously delivered GAA will be detected using a polyclonal or monoclonal anti-GAA antibody, such as the antibody used in Chen et al., Am J Hum Genet. 1987 December; 41(6): 1002-15 or Parker et al. (2007). AMP-activated protein kinase does not associate with glycogen alpha-particles from rat liver. Biochem. Biophys. Res. Commun. 362:811-815. Ten micrometer frozen sections will be cut and placed on Superfrost Plus microscope slides.

vi) Immunoblot

Immunoblot will be used to detect 3E10 and GAA immune reactive material in 3E10-GAA treated muscles and hepatic tissues. Protein isolation and immunoblot detection of 3E10 and GAA will be performed according to routine immunoblot methods. GAA will be detected with an antibody specific for this protein. Antibody detection of blotted proteins will use NBT/BCIP as a substrate. Controls will include vehicle and treated Forbes-Cori dogs and vehicle and treated homozygous wildtype dogs.

vii) Analysis of Circulating 3E10-GAA

An ELISA specific to human 3E10-GAA will be developed and validated using available anti-human GAA antibodies and horseradish peroxides conjugated anti-mouse secondary antibody (Jackson Immunoresearch). Recombinant 3E10-GAA will be diluted and used to generate a standard curve. Levels of 3E10-GAA will be determined from dilutions of

serum (normalized to ng/ml of serum) or tissue extracts (normalized to ng/mg of tissue). Controls will include vehicle and treated wildtype and Forbes-Cori dogs.

viii) Monitoring of Anti-3E10-GAA Antibody Responses

Purified 3E10-GAA used to inject Forbes-Cori dogs will be plated onto high-binding 96 well ELISA plates at 1 ug/ml in coating buffer (Pierce Biotech), allowed to coat overnight, blocked for 30 minutes in 1% nonfat drymilk (Biorad) in TBS, and rinsed three times in TBS. Two-fold dilutions of sera from vehicle and 3E10-GAA injected animals will be loaded into wells, allowed to incubate for 30 minutes at 37° C., washed three times, incubated with horseradish peroxidase (HRP)-conjugated rabbit anti-dog IgA, IgG, and IgM, allowed to incubate for 30 minutes at 37° C., and washed three times. Dog anti-3E10-GAA antibodies will be detected with TMB liquid substrate and read at 405 nm in ELISA plate reader. A polyclonal rabbit anti-dog GAA antibody, followed by HRP-conjugated goat anti-rabbit will serve as the positive control antibody reaction. Any absorbance at 405 nm greater than that of vehicle treated Forbes-Cori dogs will constitute a positive anti-3E10-GAA antibody response. Controls will include vehicle and treated wildtype dogs and Forbes-Cori dogs.

ix) Assessing Serum Enzyme Levels

Blood is collected from saphenous or jugular veins for each dog every one to three weeks for the duration of the study. Samples are tested for levels of alanine transaminase, aspartate transaminase, alkaline phosphatase, and/or creatine phosphokinase. Decrease in the elevated levels of one or more of these enzymes is indicative of reduction of some of the pathological effects of cytoplasmic glycogen accumulation.

x) Tissue Glycogen Analysis

Tissue glycogen content is assayed enzymatically using the protocol described in Yi et al. (2012). Frozen liver or muscle tissues (50-100 mg) are homogenized in ice-cold de-ionized water (20 ml water/g tissue) and sonicated three times for 20 seconds with 30-second intervals between pulses using an ultrasonicator. Homogenates are clarified by centrifugation at 12,000 g for 20 minutes at 4° C. Supernatant (20 ul) is mixed with 55 ul of water, boiled for 3 minutes and cooled to room temperature. Amyloglucosidase (Sigma) solution (25 ul diluted 1:50 into 0.1M potassium acetate buffer, pH 5.5) is added and the reaction incubated at 37° C. for 90 minutes. Samples are boiled for 3 minutes to stop the reaction and centrifuged at top speed for 3 minutes in a bench-top microcentrifuge. Supernatant (30 ul) is mixed with 1 ml of Infinity Glucose reagent (Thermo Scientific) and left at room temperature for at least 10 minutes. Absorbance at 340 nm is measured using a UV-1700 spectrophotometer. A reaction without amyloglucosidase is used for background correction for each sample. A standard curve is generated using standard glucose solutions in the reaction with Infinity Glucose reagent (0-120 uM final glucose concentration in the reaction).

xi) Survival Assessment

Those treated and untreated diseased and control dogs that are not sacrificed in the experiments described above will be monitored in a survival study. Specifically, the disease state, treatment conditions and date of death of the animals will be recorded. A survival curve will be prepared based on the results of this study.

xii) Statistical Analysis

Pairwise comparisons will employ Student's t-test. Comparisons among multiple groups will employ ANOVA. In both cases a p-value <0.05 will be considered statistically significant.

The foregoing experimental scheme will similarly be used to evaluate other chimeric polypeptides. By way of non-limiting example, this scheme will be used to evaluate chemical conjugates and fusion proteins having a GAA portion (or a fragment thereof) and an internalizing moiety portion.

Exemplary Sequences

SEQ ID NO: 1-The amino acid sequence of the human AGL protein,
isoform 1 (GenBank Accession No. NP_000019.2)
MGHSKQIRILLLNEMEKLEKTLFRLEQGYELQFRLGPTLQGKAVTVYTNYPFPGET
FNREKFRSLDWENPTEREDDSDKYCKLNLQQSGSFQYYFLQGNEKSGGGYIVVDPI
LRVGADNHVLPLDCVTLQTFLAKCLGPFDEWESRLRVAKESGYNMIHFTPLQTLG
LSRSCYSLANQLELNPDFSRPNRKYTWNDVGQLVEKLKKEWNVICITDVVYNHTA
ANSKWIQEHPECAYNLVNSPHLKPAWVLDRALWRFSCDVAEGKYKEKGIPALIEN
DHHMNSIRKIIWEDIFPKLKLWEFFQVDVNKAVEQFRRLLTQENRRVTKSDPNQHL
TIIQDPEYRRFGCTVDMNIALTTFIPHDKGPAAIEECCNWFHKRMEELNSEKHRLIN
YHQEQAVNCLLGNVFYERLAGHGPKLGPVTRKHPLVTRYFTFPFEEIDFSMEESMI
HLPNKACFLMAHNGWVMGDDPLRNFAEPGSEVYLRRELICWGDSVKLRYGNKPE
DCPYLWAHMKKYTEITATYFQGVRLDNCHSTPLHVAEYMLDAARNLQPNLYVVA
ELFTGSEDLDNVFVTRLGISSLIREAMSAYNSHEEGRLVYRYGGEPVGSFVQPCLRP
LMPAIAHALFMDITHDNECPIVHRSAYDALPSTTIVSMACCASGSTRGYDELVPHQI
SVVSEERFYTKWNPEALPSNTGEVNFQSGIIAARCAISKLHQELGAKGFIQVYVDQV
DEDIVAVTRHSPSIHQSVVAVSRTAFRNPKTSFYSKEVPQMCIPGKIEEVVLEARTIE
RNTKPYRKDENSINGTPDITVEIREHIQLNESKIVKQAGVATKGPNEYIQEIEFENLSP
GSVIIFRVSLDPHAQVAVGILRNHLTQFSPHFKSGSLAVDNADPILKIPFASLASRLTL
AELNQILYRCESEEKEDGGGCYDIPNWSALKYAGLQGLMSVLAEIRPKNDLGHPFC
NNLRSGDWMIDYVSNRLISRSGTIAEVGKWLQAMFFYLKQIPRYLIPCATDAILIGA
YTTLLDTAWKQMSSFVQNGSTFVKHLSLGSVQLCGVGKFPSLPILSPALMDVPYRL
NEITKEKEQCCVSLAAGLPHFSSGIFRCWGRDTFIALRGILLITGRYVEARNIILAFAG
TLRHGLIPNLLGEGIYARYNCRDAVWWWLQCIQDYCKMVPNGLDILKCPVSRMYP
TDDSAPLPAGTLDQPLFEVIQEAMQKHMQGIQFRERNAGPQIDRNMKDEGFNITAG
VDEETGFVYGGNRFNCGTWMDKMGESDRARNRGIPATPRDGSAVEIVGLSKSAVR
WLLELSKKNIFPYHEVTVKRHGKAIKVSYDEWNRKIQDNFEKLFHVSEDPSDLNEK
HPNLVHKRGIYKDSYGASSPWCDYQLRPNFTIAMVVAPELFTTEKAWKALEIAEK
KLLGPLGMKTLDPDDMVYCGIYDNALDNDNYNLAKGFNYHQGPEWLWPIGYFLR
AKLYFSRLMGPETTAKTIVLVKNVLSRHYVHLERSPWKGLPELTNENAQYCPFSCE
TQAWSIATILETLYDL
SEQ ID NO: 2-The amino acid sequence of the human AGL protein,
isoform 2 (GenBank Accession No. NM_000645.2)
MSLLTCAFYLGYELQFRLGPTLQGKAVTVYTNYPFPGETFNREKERSLDWENPTER
EDDSDKYCKLNLQQSGSFQYNTLQGNEKSGGGYIVVDPILRVGADNHVLPLDCVT
LQTFLAKCLGPFDEWESRLRVAKESGYNMIHFTPLQTLGLSRSCYSLANQLELNPD
FSRPNRKYTWNDVCQLAVEKLKKEWNVICITDVVYNHTAANSKWIQEHPECAYNL
VNSPHLKPAWVLDRALWRFSCDVAEGKYKEKGIPALIENDHHMNSIRKIIWEDIEP
KLKLWEFFQVDVNKAVEQFRRLLTQENRRVTKSDPNQHLTIIQDPEYRRFGCTVD
MNIALTTFIPHDKGPAAIEECCNWFHKRMEELNSEKHRLINYHQEQAVNCLLGNVF
YERLAGHGPKLGPVTRKHPLVTRYFTFPFEEIDFSMEESMIHLPNKACLMAHNGW
VMGDDPLRNFAEPGSEVYLRRELICWGDSVKLRYGNKPEDCPYLWAHMKKYTEIT
ATYFQGVRLDNCHSTPLHVAEYMLDAARNLQPNLYVVAELFTGSEDLDNVFVTRL
GISSLIREAMSAYNSHEEGRLVYRYGGEPVGSFVQPCLRPLMPAIAHALFMDITHDN
ECPIVHRSAYDALPSTTIVSMACCASGSTRGYDELVPHQISVVSEERFYTKWNPEAL
PSNTGEVNFQSGIIARCAISKLHQELGAKGFIQVYVDQVDEDIVAVTRHSPSIHQS
VVAVSRTAFRNPKTSFYSKEVPQMCIPGKIEEVYLEARTIERNTKPYRKDENSINGT
PDITVEIREHIQLNESKIVKQAGVATKGPNEYIQEIEFENLSPGSVIIFRVSLDPHAQV
AVGILRNHLTQFSRHFKSGSLAVDNADPILKIPFASLASRLTLAELNQILYRCESEEK
EDGGGCYDIPNWSALKYAGLQGLMSYLAEIRPKNDLGHPFCNNLRSGDWMIDYVS
NRLISRSGTIAEVGKWLQAMFFYLKQIPRYLIPCYFDAILIGAYTTLLDTAWKQMSS
FVQNGSTFVKHLSLGSVQLCGVGKFPSLPTLSPALMDVPYRLNEITKEKEQCCVSLA
AGLPHFSSGIFRCWGRDTFIALRGILLITGRYVEARNIILAFAGTLRHGLIPNLLGEGI
YARYNCRDAVWWWLQCIQDYCKMVPNGLDILKCPVSRMYPTDDSAPLPAGTLDQ
PLFEVIEQEAMQKHMQGIQFRERNAGQIDRNMKDEGFNITAGVDEETGFVYGGNR
FNCGTWMDKMGESDRARNRGIPATPRDGSAVEIVGLSKSAVRWLLELSKKNIFTY
HEVTNKRHGKAIKVSYDEWNRKIQDNFEKLFHVSEDPSDLNEKHPNLVHKRGINK
DSYGASSPWCDYQLRPNFTIAMVVAPELFTTEKAWKALEIAEKKLLGPLGMKTLD
PDDMVYCGIYDNALDNDNYNLAKGFNYHQGPEWLWPIGYFLRAKLYFSRLMGPE
TTAKTIVLVKNVLSRHYVHLERSPWKGLPELTNENAQYCPFSCETQAWSIATILETL
YDL
SEQ ID NO: 3-The amino acid sequence of the human AGL protein,
isoform 3 (GenBank Accession No. NM_000646.2)
MAPILSINLFIGYELQFRLGPTLQGKAVTVYTNYPFPGETFNREKFRSLDWENPTER
EDDSDKYCKLNLQQSGSFQYYFLQGNEKSGGGYIVVDPILRVGADNHVLPLDCVT
LQTFLAKCLGPFDEWESRLRVAKESGYNMIHFTPLQTLGLSRSCYSLANQLELNPD
FSRPNRKYTWNDVGQLVEKLKKEWNVICITDVVYNHTAANSKWIQEHPECAYNL
VNSPHLKPAWVLDRALWRFSCDVAEGKYKEKGIPALIENDHHMNSIRKIIWEDIFP
KLKLWEFFQVDVNKAVEQFRRLLTQENRRVTKSDPNQHLTIIQDPEYRRFGCTVD
MNIALTTFIPHDKGPAAIEECCNWFHKRMEELNSEKHRLINYMEQAVNCLLGNVF
YERLAGHGPKLGPVTRKHPLVTRYFTFPFEEIDFSMEESMIHLPNKACFLMAHNGW
VMGDDPLRNFAEPGSEVYLRRELICWGDSVKLRYGNKPEDGPYLWAHMKKYTEIT
ATYFQGVRLDNCHSTPHVAEYMLDAARNLQPNLYVVAELFTGSEDLDNVFTVTRL
GISSLIREAMSAYNSHEEGRLVYRYGGEPVGSENQPCLRPLMPAIAHALFMDITHDN
ECPIVHRSAYDALPSTTIVSMACCASGSTRGYDELVPHQISVVSEERFYTKWNPEAL
PSNTGEVNFQSGIIAARCAISKLHQELGAKGFIQVYVDQVDEDIVAVTRHSPSIHQS
VVAVSRTAFRNPKTSFYSKEVPQMCIPGKTEEVVLEARTIERNTKPYRKDENSINGT
PDITVEIREHIQLNESKIVKQAGVATKGPNEYIQEIEFENLSPGSVIIFRVSLDPHAQV
AVGILRNHLTQFSPHFKSGSLAVDNADPILKIPFASLASRLTLAELNQILYRCESEEK
EDGGGCYDIPNWSALKYAGLQGLMSVLAEIRPKNDLGHPFCNNLRSGDWMIDYNYVS
NRLISRSGTIAEVGKWLQAMFFYLKQIPRYLIPCYFDAILIGAYTTLLDTAWKQMSS
FVQNGSTFVKHLSLGSVQLCGVGKFPSLPILSPALMDVPYRLNEITKEKEQCCVSLA
AGLPHFSSGIFRCWGRDFIALRGILLITGRYVEARNIILAFAGTLRHGLIPNLLGEGI
YARYNCRDAVWWWLQCIQDYCKMVPNGLDILKCPVSRMYPTDDSAPLPAGTLDQ
PLFEVIQEAMQKHMQGIQFRERNAGPQIDRNMKDEGFNITAGVDEETGFVYGGNR
FNCGTWMDKMGESDRARNRGIPATPRDGSAVEIVGLSKSAVRWLLELSKKNIFPY
HEVTVKRHGKAIKVSYDEWNRKIQDNFEKLFHVSEDPSDLNEKHPNLVHKRGIYK
DSYGASSPWCDYQLRPNFTIAMVVAPELFTTEKAWKALEIAEKKLLGPLGMKTLD
PDDMVYCGIYDNALDNDNYNLAKGFNYHQGPEWLWPIGYFLRAKLYFSRLMGPE
TTAKTIVLVKNVLSRHYVHLERSPWKGLPELTNENAQYCPFSCETQAWSIATILETL
YDL
SEQ ID NO: 4: The amino acid sequence of the human acid alpha-
glucosidase-isoform 1 (GAA) protein (GenBank Accession No. AAA52506.1)
MGVRHPPCSHRLLAVCALVSLATAALLGHILLHDFLLVPRELSGSSPVLEETHPAH
QQGASRPGPRDAQAHPGRPRAVPTQCDVPPNSRFDCAPDKAITQEQCEARGCCYIP
AKQGLQGAQMGQPWCFFPPSYPSYKLENLSSSEMGYTATLTRTTPTFFPKDILTLRL
DVMMETENRLHFTIKDPANRRYEVPLETPHVHSRAPSPLYSVEFSEEPFGVIVRRQL
DGRVLLNTTVAPLFFADQFLQLSTSLPSQYITGLAEHLSPLMLSTSWTRITLWNRDL
APTPGANLYGSHPFYLALEDGGSAHGVFLLNSNAMDVVLQPSPALSWRSTGGILD
VYIFLGPEPKSVVQQYLDVVGYPFMPPYWGLGFHLCRWGYSSTAITRQVVENMTR
AHFPLDVQWNDLDYMDSRRDFTFNKDGFRDFPAMVQELHQGGRRYMMIVDPAIS
SSGPAGSYRPYDEGLRRGVFITNETGQPLIGKVWPGSTAFPDFTNPTALAWWEDM
VAEFHDQVPFDGMWIDMNEPSNFIRGSEDGCPNNELENPPYVPGVVGGTLQAATIC
ASSHQFLSTHYNLHNLYGLTEAIASHRALVKARGTRPFVISRSTFAGHGRYAGHWT
GDVWSSWEQLASSVPEILQFNLLGVPLVGADVCGFLGNTSEELCVRWTQLGAFYP
FMRNHNSLLSLPQEPYSFSEPAQQAMRKALTLRYALLPHLYTLFHQAHVAGETVA
RPLFLEFTKDSSTWTVDHQLLWGEALLITPVLQAGKAEVTGYFPLGTWYDLQTVPI
EALGSLPPPPAAPREPAIHSEGQWVTLPAPLDTINVHLRAGYIIPLQGPGLTTTESRQ
QPMALAVALTKGGEARGELFWDDGESLEVLERGAYTQVIFLARNNTIVNELVRVT
SEGAGLQLQKVTVLGVATAPQQVLSNGVPVSNFTYSPDTKVLDICVSLLMGEQFL
VSWC
SEQ ID NO: 5-The amino acid sequence of the human acid alpha-
glucosidase-isoform 2 (GAA) protein (GenBank Accession No. EAW89583.1)
MGVRHPPCSHRLLAVCALVSLATAALLGHILLHDFLLVPRELSGSSPVLEETHPAH
QQGASRPGPRDAQAHPGRPRAVPTQCDVPPNSRFDCAPDKAITQEQCEARGCCYIP
AKQGLQGAQMGQPWCFFPSYPSYKLENLSSSEMGYTATLTRTTPTFFPKDILTLRL
DVMMETENRLHFTIKDPANRRYEVPLETPHVHSRAPSPLYSVEFSEEPFGVIVRRQL
DGRVLLNTTVAPLFFADQFLQLSTSLPSQYITGLAEHLSPLMLSTSWTRITLWNRDL
APTPGANLYGSHPFYLALEDGGSAHGVFLLNSNAMDVVLQPSPALSWRSTGGILD
VYIFLGPEPKSVVQQYLDVVGYPFMPPYWGLGFHLCRWGYSSTAITRQVVENMTR
AHFYLDVQWNDLDYMDSRRDFTFNKDGFIWFPAMVQELHQGGRRYMMIVDPAIS
SSGPAGSYRPYDEGLRRGVHTNETGQPLIGKVWPGSTAFPDFTNPTALAWWEDM
VAEFHDQVPFDGMWIDMNEPSNFIRGSEDGCPNNELENPPYVPGVVGGTLQAATIC
ASSHQFLSTHYNLHNLYGLTEAIASHRALVKARGTRPFVISRSTFAGHGRYAGHWT
GDVWSSWEQLASSVPEILQFNLLGVPLVGADVCGFLGNTSEELCVRWTQLGAFYP
FMRNHNSLLSLPQEPYSFSEPAQQAMRKALTLRYALLPHLYTLFHQAHVAGETVA
RPLFLEFPKDSSTWTVDHQLLWGEALLITPVLQAGKAEVTGYFPLGTWYDLQTVPI
EALGSLPPPPAAPREPAIHSEGQWVTLPAPLDTINVHLRAGYIIPLQGPGLTTTESRQ
QPMALAVALTKGGEARGEILFWDDGESLEVLERGAYTQVIFLARNNTIVNELVRVT
SEGAGLQLQKVTVLGVATAPQQVLSNGVPVSNFTYSPDTKARGPRVLDICVSLLM
GEQFLVSWC
SEQ ID NO: 6 = 3E10 Variable Heavy Chain
EVQLVESGGGLVKPGGSRKLSCAASGFTFSNYGMHWVRQAPEKGLEWVAYISSGS
STIYYADTVKGRFTISRDNAKNTLFLQMTSLRSEDTAMYYCARRGLLLDYWGQGT
TLTVSS
SEQ ID NO: 7 = Linker
GGGGSGGGGSGGGGS
SEQ ID NO: 8 = 3E10 Variable Light Chain
DIVLTQSPASLAVSLGQRATISCRASKSVSTSSYSYMHWYQQKPGQPPKLLIKYASY
LESGVPARFSGSGSGTDFHLNIHPVEEEDAATYYCQHSREFPWTFGGGTKLELK
SEQ ID NO: 9-variable heavy chain CDR1 of exemplary 3E10 molecule
NYGMH
SEQ ID NO: 10-variable heavy chain CDR2 of exemplary 3E10 molecule
YISSGSSTIYYADTVKG
SEQ ID NO: 11-variable heavy chain CDR3 of exemplary 3E10 molecule
RGLLLDY
SEQ ID NO: 12-variable light chain CDR1 of exemplary 3E10 molecule
RASKSVSTSSYSYMH
SEQ ID NO: 13-variable light chain CDR2 of exemplary 3E10 molecule
YASYLES
SEQ ID NO: 14-variable light chain CDR3 of exemplary 3E10 molecule
QHSREFPWT
SEQ ID NO: 15 = exemplary mature GAA amino acid sequence
(one embodiment of mature GAA; residues 123-782)
GQPWCFFPPSYPSYKLENLSSSEMGYTATLTRTTPTFFRKDILTLRLDVMMETENRL
HFTIKDPANRRYEVPLETPHVHSRAPSPLYSVEFSEEPFGVIVRRQLDGRVLLNTTV
APLFFADQFLQLSTSLPSQYITGLAEHLSPLMLSTSWTRRITLWNRDLAPTPGANLYG
SHPFYLALEDGGSAHGVFLLNSNAMDVVLQPSPALSWRSTGGILDVYIFLGPEPKS
VVQQYLDVVGYPFMPPYWGLGFHLCRWGYSSTAITRQVVENMTRAHFPLDVQW
NDLDYMDSRRDFTFNKDGFRDFPAMVQELHQGGRRYMMIVDPAISSSGPAGSYRP
YDEGLRRGVFITNETGQPLIGKVWPGSTAFPDFTNPTALAWWEDMVAEFHDQVPF
DGMWIDMNEPSNFIRGSEDGCPNNELENPPYVPGVVGGTLQAATICASSHQFLSTH
YNLHNLYGLTEAIASHRALVKARGTRPFVISRSTFAGHGRYAGHWTGDVWSSWEQ
LASSVPEILQFNLLGVPLVGADVCGFLGNTSEELCVRWTQLGAFYPFMRNHNSLLS
LPQEPYSFSEPAQQAMRKALTLRYALLPHLYTLFHQAHVAGETVARPLFLEFPKDS
STWTVDHQLLWGEALLITPVLQAGKAEVTGYFPLGTWYDLQTVPVEA
SEQ ID NO: 16 = exemplary mature GAA amino acid sequence
(one embodiment of mature GAA; residues 288-782)
GANLYGSHPFYLALEDGGSAHGVFLLNSNAMDVVLQPSPALSWRSTGGILDVYIFL
GPEPKSVVQQYLDVVGYPFMPPYWGLGEHLCRWGYSSTAITRQVVENMTRAHFPL
DVQWNDLDYMDSRRDFTFNKDGFRDFPAMVQELHQGGRRYMMIVDPAISSSGPA
GSYRPYDEGLRRGVFITNETGQPLIGKVWPGSTAFPDFTNPTALAWWEDMVAEFH
DQVPFDGMWIDMNERSNFIRGSEDGCPNNELENPPYVPGVVGGTLQAATICASSHQ
FLSTHYNLHNLYGLTEAIASHRALVKARGTRPFVISRSTFAGHGRYAGHWTGDVW
SSWEQLASSVPEILQFNLLGVPLVGADVCGFLGNTSEELCVRWTQLGAFYPFMRNH
NSLLSLPQEPYSFSEPAQQAMRKALTLRYALLPHLNTLFHQAHVAGETVARPLFLE
FPKDSSTWTVDHQLLWGEALLITPVLQAGKAEVTGYFPLGTWYDLQTVPVEA
SEQ ID NO: 17 = Human AGL isoform 1-transcript variant 1
(GenBank Accession Number-NM_000642)
CCCGGAAGTGGGCCAGAGGTACGGTCCGCTCCCACCTGGGGCGAGTGCGCGCA
CGGCCAGGTTGGGTACCGGGTGCGCCCAGGAACCCGCGCGAGGCGAAGTCGCT
GAGACTCTGCCTGCTTCTCACCCAGCTGCCTCGGCGCTGCCCCGGTCGCTCGCC
GCCCCTCCCTTTGCCCTTCACGGCGCCCGGCCCTCCTTGGGCTGCGGCTTCTGTG
CGAGGCTGGGCAGCCAGCCCTTCCCCTTCTGTTTCTCCCCGTCCCCTCCCCCCGA
CCGTAGCACCAGAGTCGCGGGTCCTGCAGTGCCCCAGAAGCCGCACGTATAAC
TCCCTCGGCGGGTAACTCATTCGACTGTGGAGTTCTTTTAATTCTTATGAAAGAT
TTCAAATCCTCTAGAAGCCAAAATGGGACACAGTAAACAGATTCGAATTTTACT
TCTGAACGAAATGGAGAAACTGGAAAAGACCCTCTTCAGACTTGAACAAGGGT
ATGAGCTACAGTTCCGATTAGGCCCAACTTTACAGGGAAAAGCAGTTACCGTGT
ATACAAATTACCCATTTCCTGGAGAAACATTTAATAGAGAAAAATTCCGTTCTC
TGGATTGGGAAAATCCAACAGAAAGAGAAGATGATTCTGATAAATACTGTAAA
CTTAATCTGCAACAATCTGGTTCATTTCAGTATTATTTCCTTCAAGGAAATGAG
AAAAGTGGTGGAGGTTACATAGTTGTGGACCCCATTTTACGTGTTGGTGCTGAT
AATCATGTGCTACCCTTGGACTGTGTTACTCTTCAGACATTTTTAGCTAAGTGTT
TGGGACCTTTTGATGAATGGGAAAGCAGACTTAGGGTTGCAAAAGAATCAGGC
TACAACATGATTCATTTTACCCCATTGCAGACTCTTGGACTATCTAGGTCATGCT
ACTCCCTTGCCAATCAGTTAGAATTAAATCCTGACTTTTCAAGACCTAATAGAA
AGTATACCTGGAATGATGTTGGACAGCTAGTGGAAAAATTAAAAAAGGAATGG
AATGTTATTTGTATTACTGATGTTGTCTACAATCATACTGCTGCTAATAGTAAAT
GGATCCAGGAACATCCAGAATGTGCCTATAATCTTGTGAATTCTCCACACTTAA
AACCTGCCTGGGTCTTAGACAGAGCACTTTGGCGTTTGTCCTGTGATGTTGCAG
AAGGGAAATACAAAGAAAAGGGAATACCTGCTTTGATTGAAAATGATCACCAT
ATGAATTCCATCCGAAAAATAATTTGGGAGGATATTTTTCCAAAGCTTAAACTC
TGGGAATTTTTCCAAGTAGATGTCAACAAAGCGGTTGAGCAATTTAGAAGACTT
CTTACACAAGAAAATAGGCGAGTAACCAAGTCTGATCCAAACCAACACCTTAC
GATTATTCAAGATCCTGAATACAGACGGTTTGGCTGTACTGTAGATATGAACAT
TGCACTAACGACTTTCATACCACATGACAAGGGGCCAGCAGCAATTGAAGAAT
GCTGTAATTGGTTTCATAAAAGAATGGAGGAATTAAATTCAGAGAAGCATCGA
CTCATTAACTATCATCAGGAACAGGCAGTTAATTGCCTTTTGGGAAATGTGTTT
TATGAACGACTGGCTGGCCATGGTCCAAAACTAGGACCTGTCACTAGAAAGCA
TCCTTTAGTTACCAGGTATTTTACTTTCCCATTTGAAGAGATAGACTTCTCCATG
GAAGAATCTATGATTCATCTGCCAAATAAAGCTTGTTTTCTGATGGCACACAAT
GGATGGGTAATGGGAGATGATCCTCTTCGAAACTTTGCTGAACCGGGTTCAGA
AGTTTACCTAAGGAGAGAACTTATTTGCTGGGGAGACAGTGTTAAATTACGCTA
TGGGAATAAACCAGAGGACTGTCCTTATCTCTGGGCACACATGAAAAAATACA
CTGAAATAACTGCAACTTATTTCCAGGGAGTACGTCTTGATAACTGCCACTCAA
CACCTCTTCACGTAGCTGAGTACATGTTGGATGCTGCTAGGAATTTGCAACCCA
ATTTATATGTAGTAGCTGAACTGTTCACAGGAAGTGAAGATCTGGACAATGTCT
TTGTTACTAGACTGGGCATTAGTTCCTTAATAAGAGAGGCAATGAGTGCATATA
ATAGTCATGAAGAGGGCAGATTAGTTTACCGATATGGAGGAGAACCTGTTGGA
TCCTTTGTTCAGCCCTGTTTGAGGCCTTTAATGCCAGCTATTGCACATGCCCTGT
TTATGGATATTACGCATGATAATGAGTGTCCTATTGTGCATAGATCAGCGTATG
ATGCTCTTCCAAGTACTACAATTGTTTCTATGGCATGTTGTGCTAGTGGAAGTA
CAAGACGCTATGATGAATTAGTGCCTCATCAGATTTCAGTGGTTTCTGAAGAAC
GGTTTTACACTAAGTGGAATCCTGAAGCATTGCCTTCAAACACAGGTGAAGTTA
ATTTCCAAAGCGGCATTATTGCAGCCAGGTGTGCTATCAGTAAACTTCATCAGG
AGCTTGGAGCCAAGGGTTTTATTCAGGTGTATGTGGATCAAGTTGATGAAGACA
TAGTGGCAGTAACAAGACACTCACCTAGCATCCATCAGTCTGTTGTGGCTGTAT
CTAGAACTGCTTTCAGGAATCCCAAGACTTCATTTTACAGCAAGGAAGTGCCTC
AAATGTGCATCCCTGGCAAAATTGAAGAAGTAGTTCTTGAAGCTAGAACTATTG
AGAGAAACACGAAACCTTATAGGAAGGATGAGAATTCAATCAATGGAACACCA
GATATCACAGTAGAAATTAGAGAACATATTCAGCTTAAGAAAGTAAAATTGT
TAAACAAGCTGGAGTTGCCACAAAAGGGCCCAATGAATATATTCAAGAAATAG
AATTTGAAAACTTGTCTCCAGGAAGTGTTATTATATTCAGAGTTAGTCTTGATC
CACATGCACAAGTCGCTGTTGGAATTCTTCGAAATCATCTGACACAATTCAGTC
CTCACTTTAAATCTGGCAGCCTAGCTGTTGACAATGCAGATCCTATATTAAAAA
TTCCTTTTGCTTCTCTTGCCTCCAGATTAACTTTGGCTGAGCTAAATCAGATCCT
TTACCGATGTGAATCAGAAGAAAAGGAAGATGGTGGAGGGTGCTATGACATAC
CAAACTGGTCAGCCCTTAAATATGCAGGTCTTCAAGGTTTAATGTCTGTATTGG
CAGAAATAAGACCAAAGAATGACTTGGGGCATCCTTTTTGTAATAATTTGAGAT
CTGGAGATTGGATGATTGACTATGTCAGTAACCGGCTTATTTCACGATCAGGAA
CTATTGCTGAAGTTGGTAAATGGTTGCAGGCTATGTTCTTCTACCTGAAGCAGA
TCCCACGTTACCTTATCCCATGTTACTTTGATGCTATATTAATTGGTGCATATAC
CACTCTTCTGGATACAGCATGGAAGCAGATGTCAAGCTTTGTTCAGAATGGTTC
AACCTTTGTGAAACACCTTTCATTGGGTTCAGTTCAACTGTGTGGAGTAGGAAA
ATTCCCTTCCCTGCCAATTCTTTCACCTGCCCTAATGGATGTACCTTATAGGTTA
AATGAGATCACAAAAGAAAAGGAGCAATGTTGTGTTTCTCTAGCTGCAGGCTT
ACCTCATTTTTCTTCTGGTATTTTCCGCTGCTGGGGAAGGGATACTTTTATTGCA
CTTAGAGGTATACTGCTGATTACTGGACGCTATGTAGAAGCCAGGAATATTATT
TTAGCATTTGCGGGTACCCTGAGGCATGGTCTCATTCCTAATCTACTGGGTGAA
GGAATTTATGCCAGATACAATTGTCGGGATGCTGTGTGGTGGTGGCTGCAGTGT
ATCCAGGATTACTGTAAAATGGTTCCAAATGGTCTAGACATTCTCAAGTGCCCA
GTTTCCAGAATGTATCCTACAGATGATTCTGCTCCTTTGCCTGCTGGCACACTGG
ATCAGCCATTGTTTGAAGTCATACAGGAAGCAATGCAAAAACACATGCAGGGC
ATACAGTTCCGAGAAAGGAATGCTGGTCCCCAGATAGATCGAAACATGAAGGA
CGAAGGTTTTAATATAACTGCAGGAGTTGATGAAGAAACAGGATTTGTTTATGG
AGGAAATCGTTTCAATTGTGGCACATGGATGGATAAAATGGGAGAAAGTGACA
GAGCTAGAAACAGAGGAATCCCAGCCACACCAAGAGATGGGTCTGCTGTGGAA
ATTGTGGGCCTGAGTAAATCTGCTGTTCGCTGGTTGCTGGAATTATCCAAAAAA
AATATTTTCCCTTATCATGAAGTCACAGTAAAAAGACATGGAAAGGCTATAAA
GGTCTCATATGATGAGTGGAACAGAAAAATACAAGACAACTTTGAAAAGCTAT
TTCATGTTTCCGAAGACCCTTCAGATTTAAATGAAAAGCATCCAAATCTGGTTC
ACAAACGTGGCATATACAAAGATAGTTATGGAGCTTCAAGTCCTTGGTGTGACT
ATCAGCTCAGGCCTAATTTTACCATAGCAATGGTTGTGGCCCCTGAGCTCTTTA
CTACAGAAAAAGCATGGAAAGCTTTGGAGATTGCAGAAAAAAAATTGCTTGGT
CCCCTTGGCATGAAAACTTTAGATCCAGATGATATGGTTTACTGTGGAATTTAT
GACAATGCATTAGACAATGACAACTACAATCTTGCTAAAGGTTTCAATTATCAC
CAAGGACCTGAGTGGCTGTGGCCTATTGGGTATTTTCTTCGTGCAAAATTATAT
TTTTCCAGATTGATGGGCCCGGAGACTACTGCAAAGACTATAGTTTTGGTTAAA
AATGTTCTTTCCCGACATTATGTTGATCATGAGAGATCCCCTTGGAAAGGACTTC
CAGAACTGACCAATGAGAATGCCCAGTACTGTCCTTTCAGCTGTGAAACACAA
GCCTGGTCAATTGCTACTATTCTTGAGACACTTTATGATTTATAGTTTATTACAG
ATATTAAGTATGCAATTACTTGTATTATAGGATGCAAGGTCATCATATGTAAAT
GCCTTATATGCACAGGCTCAAGTTGTTTTAAAAATCTCATTTATTATAATATTGA
TGCTCAATTAGGTAAGATTGTAAAAGCATTGATFTTTTTTAATGTACAGAGGTA
GATTTCAATTTGAATCAGAAAGAAATATCATTACCAATGAAATGTGTTTGAGTT
CAGTAAGAATTATTCAAATGCCTAGAAATCCATAGTTTGGAAAAGAAAAATCA
TGTCATCTTCTATTTGTACAGAAATGAAAATAAAATATGAAAATAATGAAAGA
AATGAAAAGATAGCTTTTAATTGTGGTATATATAATCTTCAGTAACAATACATA
CTGAATACGCTGTGGTTCATTAATATTAACACCACGTACTATAGTATTCTTAAT
ACAGTGCTCACTGCATTTAATAAATATTTAATAAATGATGAATGATAGAAGTTT
CCATCTACAATATATGTTCCTAAATGGAGCACAGATGTTCAAACTATGCTTTCA
TTTTTTCACTGATATATTAATTTTTGTGTAATGAATGCCAACAGTATATTTTATA
TGATTTACTTATGTGAGGAAACATGCAAAGCATTAGGAAATTTATTTCCTAAAA
ACAGTTTTGTAAAATTAGTATTGAGTTCTATTGAGTATTATAAGATAGCTTACA
TTTTCAAAATGGAAATTGTCGGTCATATTTCTAGAACTTTAAAGAAAAAAGAAT
GTTATATTAGTTTTCTAAAACTCAACTATCTTTAGTCATGTTCAAAAATCTATTG
CTAGATCATAGTAGATACTGGTTTTCTATTAACTCAAAACCTACATTGACAAGT
TTAACATTGAGAAGAATCTTAACAAAAATATGGATATGAATTCAGTAGATATCT
TAAATTCAATAAAATCACTGGAAGTTTTTCATGATAACTTATTTTAAGATGCCTT
AAAAATCTTAAAGTCACAAAAGGAAAAAGGTTTTTAACATTTACATGAGTTAA
CATTTTTTCATAGAACTTATTTCCTAGATAGAATTTTTTACTGTTTTTTACTGTTT
TCTTAAGAAAACAGTTAAATCATTATGCATTCAGTTGGAAGAAAGTAGTGGCA
AGAATTCTTTCATTGCTATATAATATTCAGTGGCTCATTTATACCTAATAAAATA
ATGGTATTTTAAAATAATGCTACTTTCAAAGTAGCATTTTTTTAGTTAGTTTACA
GGTTACATACCCAAAACCTTAACTATGACTAAGAAATTAAAGAAGAAAACCAG
CAAACTAAAACTTCTGGGCAGCAAAAATATATAAATGCTTCAGATGTCAAATA
CCCATGCTTGAAAGCTCGTGTAATTTACTTTAAGATTATCTGCCTGCTCTTCTTC
AAAGCTGACCTTGCTTTAGAAATAGTTTTAACTAGCTTAGTTTTCTGGTTTCCAA
AACTAAAATAGATTAAATCCTACAAATTTAAGGACAGTTGTGACAGTAATCTG
ACCACTATCTATAAATACATTGGACATTGGTTTCCAAATCTCCCTTTCTTCTTCA
GTTCCTTCCTTGTTCAATATATACCCTTCTCTAAACTGTGCGGGTAAAAGGAAT
GACTGTCCTTGAGAGAACCATTAGTTTATCAAAGGTTTATGTAGTTTTGTTGCTG
TACCCTAACTTTGATATTCAGGGAGGTAGGAAAGGTAACAGAAAACCAGCATA
TTTAATCAAAGCAAGAAGTAATCGCTGACAGTTAAATGTGACCAAAAAAATTA
AAAGTTCACAATTTTTTTAATGTAGCCATTTGGGGTTATCTCTAGTAAGGCAGA
TACCCACGTTGGTAAATTTTTAGGATATTGTGTTGCACTAGAAAACTAAGTGGT
TCATATTTCTAATGAGGAAGATTAATGAAAGAACATTGTTATATTCTGCGTGGT
ATATTTTAAAGTTTAAGAAGGCATGTTAAACATTATTTCCTCTATGGTAGTTAA
AATACAGAATTAGATTTTTAACAGGTGTCATTTGACTAAACGTTTCGGTAGAAT
GCTTCATACTTGAGTGATGCTGGATAAGGTATTGTATTTCAACAATGGACTATG
CCTTGGTTTTTCACTAATCAAAATCAAAATTACTCTTTAACATGATAAATGAATT
TACCAGTTTAGTATGCTGTGGTATTTTAATAAGTTTTCAAAGATAATTGGGAAA
ACATGAGACTGGTCATATTGATGAATATTGTAACATGTGAATTGTGATCCATTT
CTGATATGTCTTGAACTACTGTGTCTAGTGGGCAAATGTCATTGTTACCTCTGTG
TGTTAAGAAAATAAAAATATTTTCTAAAGGTCTGT
SEQ ID NO: 18 = Human AGL isoform 1-transcript variant 2
(GenBank Accession Number-NM_900644)
CTGTCTACGGCAGCTATTCCAGAGGCAACAACTGCTTCCCTCTGTTCTCATCTCC
CCATTGGTGGCTGGCGACCCGAATTTGGGAATGGGGAGATTGCCCACCTGTTAT
CTTTGAGCAGACTAATCTCTTAAGCCAAAATGGGACACAGTAAACAGATTCGA
ATTTTACTTCTGAACGAAATGGAGAAACTGGAAAAGACCCTCTTCAGACTTGAA
CAAGGGTATGAGCTACAGTTCCGATTAGGCCCAACTTTACAGGGAAAAGCAGT
TACCGTGTATACAAATTACCCATTTCCTGGAGAAACATTTAATAGAGAAAAATT
CCGTTCTCTGGATTGGGAAAATCCAACAGAAAGAGAAGATGATTCTGATAAAT
ACTGTAAACTTAATCTGCAACAATCTGGTTCATTTCAGTATTATTTCCTTCAAGG
AAATGAGAAAAGTGGTGGAGGTTACATAGTTGTGGACCCCATTTTACGTGTTGG
TGCTGATAATCATGTGCTACCCTTGGACTGTGTTACTCTTCAGACATTTTTAGCT
AAGTGTTTGGGACCTTTTGATGAATGGGAAAGCAGACTTAGGGTTGCAAAAGA
ATCAGGCTACAACATGATTCATTTTACCCCATTGCAGACTCTTGGACTATCTAG
GTCATGCTACTCCCTTGCCAATCAGTTAGAATTAAATCCTGACTTTTCAAGACCT
AATAGAAAGTATACCTGGAATGATGTTGGACAGCTAGTGGAAAAATTAAAAAA
GGAATGGAATGTTATTTGTATTACTGATGTTGTCTACAATCATACTGCTGCTAAT
AGTAAATGGATCCAGGAACATCCAGAATGTGCCTATAATCTTGTGAATTCTCCA
CACTTAAAACCTGCCTGGGTCTTAGACAGAGCACTTTGGCGTTTCTCCTGTGAT
GTTGCAGAAGGGAAATACAAAGAAAAGGGAATACCTGCTTTGATTGAAAATGA
TCACCATATGAATTCCATCCGAAAAATAATTTGGGAGGATATTTTTCCAAAGCT
TAAACTCTGGGAATTTTTCCAAGTAGATGTCAACAAAGCGGTTGAGCAATTTAG
AAGACTTCTTACACAAGAAAATAGGCGAGTAACCAAGTCTGATCCAAACCAAC
ACCTTACGATTATTCAAGATCCTGAATACAGACGGTTTGGCTGTACTGTAGATA
TGAACATTGCACTAACGACTTTCATACCACATGACAAGGGGCCAGCAGCAATT
GAAGAATGCTGTAATTGGTTTCATAAAAGAATGGAGGAATTAAATTCAGAGAA
GCATCGACTCATTAACTATCATCAGGAACAGGCAGTTAATTGCCTTTTGGGAAA
TGTGTTTTATGAACGACTGGCTGGCCATGGTCCAAAACTAGGACCTGTCACTAG
AAAGCATCCTTTAGTTACCAGGTATTTTACTTTCCCATTTGAAGAGATAGACTTC
TCCATGGAAGAATCTATGATTCATCTGCCAAATAAAGCTTGTTTTCTGATGGCA
CACAATGGATGGGTAATGGGAGATGATCCTCTTCGAAACTTTGCTGAACCGGGT
TCAGAAGTTTACCTAAGGAGAGAACTTATTTGCTGGGGAGACAGTGTTAAATTA
CGCTATGGGAATAAACCAGAGGACTGTCCTTATCTCTGGGCACACATGAAAAA
ATACACTGAAATAACTGCAACTTATTTCCAGGGAGTACGTCTTGATAACTGCCA
CTCAACACCTCTTCACGTAGCTGAGTACATGTTGGATGCTGCTAGGAATTTGCA
ACCCAATTTATATGTAGTAGCTGAACTGTTCACAGGAAGTGAAGATCTGGACA
ATGTCTTMTTACTAGACTGGGCATTAGTTCCTTAATAAGAGAGGCAATGAGTG
CATATAATAGTCATGAAGAGGGCAGATTAGTTTACCGATATGGAGGAGAACCT
GTTGGATCCTTTGTTCAGCCCTGTTTGAGGCCTTTAATGCCAGCTATTGCACATG
CCCTGTTTATGGATATTACGCATGATAATGAGTGTCCTATTGTGCATAGATCAG
CGTATGATGCTCTTCCAAGTACTACAATTGTTTCTATGGCATGTTGTGCTAGTGG
AAGTACAAGAGGCTATGATGAATTAGTGCCTCATCAGATTTCAGTGGTTTCTGA
AGAACGGTTTTACACTAAGTGGAATCCTGAAGCATTGCCTTCAAACACAGGTG
AAGTTAATTTCCAAAGCGGCATTATTGCAGCCAGGTGTGCTATCAGTAAACTTC
ATCAGGAGCTTGGAGCCAAGGGTTTTATTCAGGTGTATGTGGATCAAGTTGATG
AAGACATAGTGGCAGTAACAAGACACTCACCTAGCATCCATCAGTCTGTTGTG
GCTGTATCTAGAACTGCTTTCAGGAATCCCAAGACTTCATTTTACAGCAAGGAA
GTGCCTCAAATGTGCATCCCTGGCAAAATTGAAGAAGTAGTTCTTGAAGCTAGA
ACTATTGAGAGAAACACGAAACCTTATAGGAAGGATGAGAATTCAATCAATGG
AACACCAGATATCACAGTAGAAATTAGAGAACATATTCAGCTTAATGAAAGTA
AAATTGTTAAACAAGCTGGAGTTGCCACAAAAGGGCCCAATGAATATATTCAA
GAAATAGAATTTGAAAACTTGTCTCCAGGAAGTGTTATTATATTCAGAGTTAGT
CTTGATCCACATGCACAAGTCGCTGTTGGAATTCTTCGAAATCATCTGACACAA
TTCAGTCCTCACTTTAAATCTGGCAGCCTAGCTGTTGACAATGCAGATCCTATA
TTAAAAATTCCTTTTGCTTCTCTTGCCTCCAGATTAACTTTGGCTGAGCTAAATC
AGATCCTTTACCGATGTGAATCAGAAGAAAAGGAAGATGGTGGAGGGTGCTAT
GACATACCAAACTGGTCAGCCCTTAAATATGCAGGTCTTCAAGGTTTAATGTCT
GTATTGGCAGAAATAAGACCAAAGAATGACTTGGGGCATCCTTTTTGTAATAAT
TTGAGATCTGGAGATTGGATGATTGACTATGTCAGTAACCGGCTTATTTCACGA
TCAGGAACTATTGCTGAAGTTGGTAAATGGTTGCAGGCTATGTTCTTCTACCTG
AAGCAGATCCCACGTTACCTTATCCCATGTTACTTTGATGCTATATTAATTGGTG
CATATACCACTCTTCTGGATACAGCATGGAAGCAGATGTCAAGCTTTGTTGAGA
ATGGTTCAACCTTTGTGAAACACCTTTCATTGGGTTCAGTTCAACTGTGTGGAG
TAGGAAAATTCCCTTCCCTGCCAATTCTTTCACCTGCCCTAATGGATGTACCTTA
TAGGTTAAATGAGATCACAAAAGAAAAGGAGCAATGTTGTGTTTCTCTAGCTG
CAGGCTTACCTCATTTTTCTTCTGGTATTTTCCGCTGCTGGGGAAGGGATACTTT
TATTGCACTTAGAGGTATACTGCTGATTACTGGACGCTATGTAGAAGCCAGGAA
TATTATTTTAGCATTTGCGGGTACCCTGAGGCATGGTCTCATTCCTAATCTACTG
GGTGAAGGAATTTATGCCAGATACAATTGTCGGGATGCTGTGTGGTGGTGGCTG
CAGTGTATCCAGGATTACTGTAAAATGGTTCCAAATGGTCTAGACATTCTCAAG
TGCCCAGTTTCCAGAATGTATCCTACAGATGATTCTGCTCCTTTGCCTGCTGGCA
CACTGGATCAGCCATTGTTTGAAGTCATACAGGAAGCAATGCAAAAACACATG
CAGGGCATACAGTTCCGAGAAAGGAATGCTGGTCCCCAGATAGATCGAAACAT
GAAGGACGAAGGTTTTAATATAACTGCAGGAGTTGATGAAGAAACAGGATTTG
TTTATGGAGGAAATCGTTTCAATTGTGGCACATGGATGGATAAAATGGGAGAA
AGTGACAGAGCTAGAAACAGAGGANTCCCAGCCACACCAAGAGATGGGTCTGC
TGTGGAAATTGTGGGCCTGAGTAAATCTGCTGTTCGCTGGTTGCTGGAATTATC
CAAAAAAAATATTTTCCCTTATCATGAAGTCACAGTAAAAAGACATGGAAAGG
CTATAAAGGTCTCATATGATGAGTGGAACAGAAAAATACAAGACAACTTTGAA
AAGCTATTTCATGTTTCCGAAGACCCTTCAGATTTAAATGAAAAGCATCCAAAT
CTGGTTCACAAACGTGGCATATACAAAGATAGYfATGGAGCTTCAAGTCCTTGG
TGTGACTATCAGCTCAGGCCTAATTTTACCATAGCAATGGTTGTGGCCCCTGAG
CTCTTTACTACAGAAAAAGCATGGAAAGCTTTGGAGATTGCAGAAAAAAAATT
GCTTGGTCCCCTTGGCATGAAAACTTTAGATCCAGATGATATGGTTTACTGTGG
AATTTATGACAATGCATTAGACAATGACAACTACAATCTTGCTAAAGGTTTCAA
TTATCACCAAGGACCTGAGTGGCTGTGGCCTATTGGGTATTTTCTTCGTGCAAA
ATTATATTTTTCCAGATTGATGGGCCCGGAGACTACTGCAAAGACTATAGTTTT
GGTTAAAAATGTTCTTTCCCGACATTATGTTCATCTTGAGAGATCCCCTTGGAA
AGGACTTCCAGAACTGACCAATGAGAATGCCCAGTACTGTCCTTTCAGCTGTGA
AACACAAGCCTGGTCAATTGCTACTATTCTTGAGACACTTTATGATTTATAGTTT
ATTACAGATATTAAGTATGCAATTACTTGTATTATAGGATGCAAGGTCATCATA
TGTAAATGCCTTATATGCACAGGCTCAAGTTGTTTTAAAAATCTCATTTATTATA
ATATTGATGCTCAATTAGGTAAGATTGTAAAAGCATTGATTTTTTTTAATGTAC
AGAGGTAGATTTCAATTTGAATCAGAAAGAAATATCATTACCAATGAAATGTG
TTTGAGTTCAGTAAGAATTATTCAAATGCCTAGAAATCCATAGTTTGGAAAAGA
AAAATCATGTCATCTTCTATTTGTACAGAAATGAAAATAAAATATGAAAATAAT
GAAAGAAATGAAAAGATAGCTTTTAATTGTGGTATATATAATCTTCAGTAACAA
TACATACTGAATACGCTGTGGTTCATTAATATTAACACCACGTACTATAGTATT
CTTAATACAGTGCTCACTGCATTTAATAAATATTTAATAAATGATGAATGATAG
AAGTTTCCATCTACAATATATGTTCCTAAATGGAGCACAGATGTTCAAACTATG
CTTTCATTTTTTCACTGATATATTAATTTTTGTGTAATGAATGCCAACAGTATAT
TTTATATGATTTACTTATGTGAGGAAACATGCAAAGCATTAGGAAATTTATTTC
CTAAAAACAGTTTTGTAAAATTAGTATTGAGTTcTATTGAGTATTATAAGATAG
CTTACATTTTCAAAATGGAAATTGTCGGTCATATTTCTAGAACTTTAAAGAAAA
AAGAATGTTATATTAGTTTTCTAAAACTCAACTATCTTTAGTCATGTTCAAAAAT
CTATTGCTAGATCATAGTAGATACTGGTTTTCTATTAACTCAAAACCTACATTG
ACAAGTTTAACATTGAGAAGAATCTTAACAAAAATAGGATATGAATTCAGTA
GATATCTTAAATTCAATAAAATCACTGGAAGTTTTTCATGATAACTTATTTTAA
GATGCCTTAAAAATCTTAAAGTCACAAAAGGAAAAAGGTTTTTAACATTTACAT
GAGTTAACATTTTTTCATAGAACTTATTTCCTAGATAGAATTTTTTACTGTTTTTT
ACTGTTTTCTTAAGAAAACAGTTAAATCATTATGCATTCAGTTGGAAGAAAGTA
GTGGCAAGAATTCTTTCATTGCTATATAATATTCAGTGGCTCATTTATACCTAAT
AAAATAATGGTATTTTAAAATAATGCTACTTTCAAAGTAGCATTTTTTTAGTTA
GTTTACAGGTTACATACCCAAAACCTTAACTATGACTAAGAAATTAAAGAAGA
AAACCAGCAAACTAAAACTTCTGGGCAGCAAAAATATATATGCTTCAGATG
TCAAATACCCATGCTTGAAAGCTCGTGTAATTTACTTTAAGATTATCTGCCTGCT
CTTCTTCAAAGCTGACCTTGCTTTAGAAATAGTTTTAACTAGCTTAGTTTTCTGG
TTTCCAAAACTAAAATAGATTAAATCCTACAAATTTAAGGACAGTTGTGACAGT
AATCTGACCACTATCTATAAATACATTGGACATTGGTTTCCAAATCTCCCTTTCT
TCTTCAGTTCCTTCCTTGTTCAATATATACCCTTCTCTAAACTGTGCGGGTAAAA
GGAATGACTGTCCTTGAGAGAACCATTAGTTTATCAAAGGTTTATGTAGTTTTG
TTGCTGTACCCTAACTTTGATATTCAGGGAGGTAGGAAAGGTCAGAAAACC
AGCATATTTAATCAAAGCAAGAAGTAATCGCTGACAGTTAAATGTGACCAAAA
AAATTAAAAGTTCACAATTTTTTTAATGTAGCCATTTGGGGTTATCTCTAGTAA
GGCAGATACCCACGTTGGTAAATTTTTAGGATATTGTGTTGCACTAGAAAACTA
AGTGGTTCATATTTCTAATGAGGAAGATTAATGAAAGAACATTGTTATATTCTG
CGTGGTATATTTTAAAGTTTAAGAAGGCATGTTAAACATTATTTCCTCTATGGT
AGTTAAAATACAGAATTAGATTTTTAACAGGTGTCATTTGACTAAACGTTTCGG
TAGAATGCTTCATACTTGAGTGATGCTGGATAAGGTATTGTATTTCAACAATGG
ACTATGCCTTGGTTTTTCACTAATCAAAATCAAAATTACTCTTTAACATGATAA
ATGAATTTACCAGTTTAGTATGCTGTGGTATTTTAATAAGTTTTCAAAGATAATT
GGGAAAACATGAGACTGGTCATATTGATGAATATTGTAACATGTGAATTGTGAT
CCATTTCTGATATGTCTTGAACTACTGTGTCTAGTGGGCAAATGTCATTGTTACC
TCTGTGTGTTAAGAAAATAAAAATATTTTCTAAAGGTCTGT
SEQ ID NO: 19 = Human AGL isoform 1-transcript variant 3
(GenBank Accession Number-NM_000643)
CTGTCTACGGCAGCTATTCCAGAGGCAACAACTGCTTCCCTCTGTTCTCATCTCC
CCATTGGTGGCTGGCGACCCGAATTTGGGAATGGGGAGATTGCCCACCTGTTAT
CTTTGAGCAGACTAATCTCTTGGGTAACTCATTCGACTGTGGAGTTCTTTTAATT
CTTATGAAAGATTTCAAATCCTCTAGAAGCCAAAATGGGACACAGTAAACAGA
TTCGAATTTTACTTCTGAACGAAATGGAGAAACTGGAAAAGACCCTCTTCAGAC
TTGAACAAGGGTATGAGCTACAGTTCCGATTAGGCCCAACTTTACAGGGAAAA
GCAGTTACCGTGTATACAAATTACCCATTTCCTGGAGAAACATTTAATAGAGAA
AAATTCCGTTCTCTGGATTGGGAAAATCCAACAGAAAGAGAAGATGATTCTGA
TAAATACTGTAAACTTAATCTGCAACAATCTGGTTCATTTCAGTATTATTTCCTT
CAAGGAAATGAGAAAAGTGGTGGAGGTTACATAGTTGTGGACCCCATTTTACG
TGTTGGTGCTGATAATCATGTGCTACCCTTGGACTGTGTTACTCTTCAGACATTT
TTAGCTAAGTGTTTGGGACCTTTTGATGAATGGGAAAGCAGACTTAGGGTTGCA
AAAGAATCAGGCTACAACATGATTCATTTTACCCCATTGCAGACTCTTGGACTA
TCTAGGTCATGCTACTCCCTTGCCAATCAGTTAGAATTAAATCCTGACTTTTCAA
GACCTAATAGAAAGTATACCTGGAATGATGTTGGACAGCTAGTGGAAAAATTA
AAAAAGGAATGGAATGTTATTTGTATTACTGATGTTGTCTACAATCATACTGCT
GCTAATAGTAAATGGATCCAGGAACATCCAGAATGTGCCTATAATCTTGTGAAT
TCTCCACACTTAAAACCTGCCTGGGTCTTAGACAGAGCACTTTGGCGTTTCTCCT
GTGATGTTGCAGAAGGGAAATACAAAGAAAAGGGAATACCTGCTTTGATTGAA
AATGATCACCATATGAATTCCATCCGAAAAATAATTTGGGAGGATATTTTTCCA
AAGCTTAAACTCTGGGAATTTTTCCAAGTAGATGTCAACAAAGCGGTTGAGCA
ATTTAGAAGACTTCTTACACAAGAAAATAGGCGAGTAACCAAGTCTGATCCAA
ACCAACACCTTACGATTATTCAAGATCCTGAATACAGACGGTTTGGCTGTACTG
TAGATATGAACATTGCACTAACGACTTTCATACCAcATGACAAGGGGCCAGCA
GCAATTGAAGAATGCTGTAATTGGTTTCATAAAAGAATGGAGGAATTAAATTC
AGAGAAGCATCGACTCATTAACTATCATCAGGAACAGGCAGTTAATTGCCTTTT
GGGAAATGTGTTTTATGAACGACTGGCTGGCCATGGTCCAAAACTAGGACCTGT
CACTAGAAAGCATCCTTTAGTTACCAGGTATTTTACTTTCCCATTTGAAGAGAT
AGACTTCTCCATGGAAGAATCTATGATTCATCTGCCAAATAAAGCTTGTTTTCT
GATGGCACACAATGGATGGGTAATGGGAGATGATCCTCTTCGAAACTTTGCTG
AACCGGGTTCAGAAGTTTACCTAAGGAGAGAACTTATTTGCTGGGGAGACAGT
GTTAAATTACGCTATGGGAATAAACCAGAGGACTGTCCTTATCTCTGGGCACAC
ATGAAAAAATACACTGAAATAACTGCAACTTATTTCCAGGGAGTACGTCTTGAT
AACTGCCACTCAACACCTCTTCACGTAGCTGAGTACATGTTGGATGCTGCTAGG
AATTTGCAACCCAATTTATATGTAGTAGCTGAACTGTTCACAGGAAGTGAAGAT
CTGGACAATGTCTTTGTTACTAGACTGGGCATTAGTTCCTTAATAAGAGAGGCA
ATGAGTGCATATAATAGTCATGAAGAGGGCAGATTAGTTTACCGATATGGAGG
AGAACCTGTTGGATCCTTTGTTCAGCCCTGTTTGAGGCCTTTAATGCCAGCTATT
GCACATGCCCTGTTTATGGATATTACGCATGATAATGAGTGTCCTATTGTGCAT
AGATCAGCGTATGATGCTCTrCCAAGTACTACAATTGTTTCTATGGCATGTTGT
GCTAGTGGAAGTACAAGAGGCTATGATGAATTAGTGCCTCATCAGATTTCAGTG
GTTTCTGAAGAACGGTTTTACACTAAGTGGAATCCTGAAGCATTGCCTTCAAAC
ACAGGTGAAGTTAATTTCCAAAGCGGCATTATTGCAGCCAGGTGTGCTATCAGT
AAACTTCATCAGGAGCTTGGAGCCAAGGGTTTTATTCAGGTGTATGTGGATCAA
GTTGATGAAGACATAGTGGCAGTAACAAGACACTCACCTAGCATCCATCAGTC
TGTTGTGGCTGTATCTAGAACTGCTTTCAGGAATCCCAAGACTTCATTTTACAG
CAAGGAAGTGCCTCAAATGTGCATCCCTGGCAAAATTGAAGAAGTAGTTCTTG
AAGCTAGAACTATTGAGAGAAACACGAAACCTTATAGGAAGGATGAGAATTCA
ATCAATGGAACACCAGATATCACAGTAGAAATTAGAGAACATATTCAGCTTAA
TGAAAGTAAAATTGTTAAACAAGCTGGAGTTGCCACAAAAGGGCCCAATGAAT
ATATTCAAGAAATAGAATTTGAAAACTTGTCTCCAGGAAGTGTTATTATATTCA
GAGTTAGTCTTGATCCACATGCACAAGTCGCTGTTGGAATTCTTCGAAATCATC
TGACACAATTCAGTCCTCACTTTAAATCTGGCAGCCTAGCTGTTGACAATGCAG
ATCCTATATTAAAAATTCCTTTTGCTTCTCTTGCCTCCAGATTAACTTTGGCTGA
GCTAAATCAGATCCTTTACCGATGTGAATCAGAAGAAAAGGAAGATGGTGGAG
GGTGCTATGACATACCAAACTGGTCAGCCCTTAAATATGCAGGTCTTCAAGGTT
TAATGTCTGTATTGGCAGAAATAAGACCAAAGAATGACTTGGGGCATCCTTTTT
GTAATAATTTGAGATCTGGAGATTGGATGATTGACTATGTCAGTAACCGGCTTA
TTTCACGATCAGGAACTATTGCTGAAGTTGGTAAATGGTTGCAGGCTATGTTCT
TCTACCTGAAGCAGATCCCACGTTACCTTATCCCATGTTACTTTGATGCTATATT
AATTGGTGCATATACCACTCTTCTGGATACAGCATGGAAGCAGATGTCAAGCTT
TCTTTCAGAATGGTTCAACCTTTGTGAAACACCTTTCATTGGGTTCAGTTCAACTG
TGTGGAGTAGGAAAATTCCCTTCCCTGCCAATTCTTTCACCTGCCCTAATGGAT
GTACCTTATAGGTTAAATGAGATCACAAAAGAGGAGCAATGTTGTGTTTCT
CTAGCTGCAGGCTTACCTCATTTTTCTTCTGGTATTTTCCGCTGCTGGGGAAGGG
ATACTTTTATTGCACTTAGAGGTATACTGCTGATTACTGGACGCTATGTAGAAG
CCAGGAATATTATTTTAGCATTTGCGGGTACCCTGAGGCATGGTCTCATTCCTA
ATCTACTGGGTGAAGGAATTTATGCCAGATACAATTGTCGGGATGCTGTGTGGT
GGTGGCTGCAGTGTATCCAGGATTACTGTAAAATGGTTCCAAATGGTCTAGACA
TTCTCAAGTGCCCAGTTTCCAGAATGTATCCTACAGATGATTCTGCTCCTTTGCC
TGCTGGCACACTGGATCAGCCATTGTTTGAAGTCATACAGGAAGCAATGCAAA
AACACATGCAGGGCATACAGTTCCGAGAAAGGAATGCTGGTCCCCAGATAGAT
CGAAACATGAAGGACGAAGGTTTTAATATAACTGCAGGAGTTGATGAAGAAAC
AGGATTTGTTTATGGAGGAAATCGTTTCAATTGTGGCACATGGATGGATAAAAT
GGGAGAAAGTGACAGAGCTAGAAACAGAGGAATCCCAGCCACACCAAGAGAT
GGGTCTGCTGTGGAAATTGTGGGCCTGAGTAAATCTGCTGTTCGCTGGTTGCTG
GAATTATCCAAAAAAAATATTTTCCCTTATCATGAAGTCACAGTAAAAAGACAT
GGAAAGGCTATAAAGGTCTCATATGATGAGTGGAACAGAAAAATACAAGACA
ACTTTGAAAAGCTATTTCATGTTCCGAAGACCCTTCAGATTTAAATGAAAAGC
ATCCAAATCTGGTTCACAAACGTGGCATATACAAAGATAGTTATGGAGCTTCAA
GTCCTTGGTGTGACTATCAGCTCAGGCCTAATTTTACCATAGCAATGGTTGTGG
CCCCTGAGCTCTTTACTACAGAAAAAGCATGGAAAGCTTTGGAGATTGCAGAA
AAAAAATTGCTTGGTCCCCTTGGCATGAAAACTTTAGATCCAGATGATATGGTT
TACTGTGGAATTTATGACAATGCATTAGACAATGACAACTACAATCTTGCTAAA
GGTTTCAATTATCACCAAGGACCTGAGTGGCTGTGGCCTATTGGGTATTTTCTTC
GTGCAAAATTATATTTTTCCAGATTGATGGGCCCGGAGACTACTGCAAAGACTA
TAGTTTTGGTTAAAAATGTTCTTTCCCGACATTATGTTCATCTTGAGAGATCCCC
TTGGAAAGGACTTCCAGAACTGACCAATGAGAATGCCCAGTACTGTCCTTTCAG
CTGTGAAACACAAGCCTGGTCAATTGCTACTATTCTTGAGACACTTTATGATTT
ATAGTTTATTACAGATATTAAGTATGCAATTACTTGTATTATAGGATGCAAGGT
CATCATATGTAAATGCCTTATATGCACAGGCTCAAGTTGTTTTAAAAATCTCAT
TTATTATAATATTGATGCTCAATTAGGTAAGATTGTAAAAGCATTGATTTTTTTT
AATGTACAGAGGTAGATTTCAATTTGAATCAGAAAGAAATATCATTACCAATG
AAATGTGTTTGAGTTCAGTAAGAATTATTCAAATGCCTAGAAATCCATAGTTTG
GAAAAGAAAAATCATGTCATCTTCTATTTGTACAGAAATGAAAATAAAATATG
AAAATAATGAAAGAAATGAAAAGATAGCTTTTAATTGTGGTATATATAATCTTC
AGTAACAATACATACTGAATACGCTGTGGTTCATTAATATTAACACCACGTACT
ATAGTATTCTTAATACAGTGCTCACTGCATTTAATAAATATTTAATAAATGATG
AATGATAGAAGTTTCCATCTACAATATATGTTCCTAAATGGAGCACAGATGTTC
AAACTATGCTTTCATTTTTTCACTGATATATTAATTTTTGTGTAATGAATGCCAA
CAGTATATTTTATATGATTTACTTATGTGAGGAAACATGCAAAGCATTAGGAAA
TTTATTTCCTAAAAACAGTTTTGTAAAATTAGTATTGAGTTCTATTGAGTATTAT
AAGATAGCTTACATTTTCAAAATGGAAATTGTCGGTCATATTTCTAGAACTTTA
AAGAAAAAAGAATGTTATATTAGTTTTCTAAAACTCAACTATCTTTAGTCATGT
TCAAAAATCTATTGCTAGATCATAGTAGATACTGGTTTTCTATTAACTCAAAAC
CTACATTGACAAGTTTAACATTGAGAAGAATCYLAACAAAAATATGGATATGA
ATTCAGTAGATATCTTAAATTCAATAAAATCACTGGAAGTTTTTCATGATAACT
TATTTTAAGATGCCTTAAAAATCTTAAAGTCACAAAAGGAAAAAGGTTTTTAAC
ATTTACATGAGTTAACATTTTTTCATAGAACTTATTTCCTAGATAGAATTTTTTA
CTGTTTTTTACTGTTTTCTTAAGAAAACAGTTAAATCATTATGCATTCAGTTGGA
AGAAAGTAGTGGCAAGAATTCTTTCATTGCTATATAATATTCAGTGGCTCATTT
ATACCTAATAAAATAATGGTATTTTAAAATAATGCTACTTTCAAAGTAGCATTT
TTTTAGTTAGTTTACAGGTTACATACCCAAAACCTTAACTATGACTAAGAAATT
AAAGAAGAAAACCAGCAAACTAAAACTTCTGGGCAGCAAAAATATATAAATGC
TTCAGATGTCAAATACCCATGCTTGAAAGCTCGTGTAATTTACTTTAAGATTAT
CTGCCTGCTCTTCTTCAAAGCTGACCTTGCTTTAGAAATAGTTTTAACTAGCTTA
GTTTTCTGGTTTCCAAAACTAAAATAGATTAAATCCTACAAATTTAAGGACAGT
TGTGACACTTAATCTGACCACTATCTATAAATACATTGGACATTGGTTTCCAAAT
CTCCCTTTCTTCTTCAGTTCCTTCCTTGTTCAATATATACCCTTCTCTAAACTGTG
CGGGTAAAAGGAATGACTGTCCTTGAGAGAACCATTAGTTTATCAAAGGTTTAT
GTAGTTTTGTTGCTGTACCCTAACTTTGATATTCAGGGAGGTAGGAAAGGTAAC
AGAAAACCAGCATATTTAATCAAAGCAAGAAGTAATCGCTGACAGTTAAATGT
GACCAAAAATTAAAAGTTCACAATTTTTTTAATGTAGCCATTTGGGGTTATC
TCTAGTAAGGCAGATACCCACGTTGGTAAATTTTTAGGATATTGTGTTGCACTA
GAAAACTAAGTGGTTCATATTTCTAATGAGGAAGATTAATGAAAGAACATTGTT
ATATTCTGCGTGGTATATTTTAAAGTTTAAGAAGGCATGTTAAACATTATTTCCT
CTATGGTAGTTAAAATACAGAATTAGATTTTTAACAGGTGTCATTTGACTAAAC
GTTTCGGTAGAATGCTTCATACTTGAGTGATGCTGGATAAGGTATTGTATTTCA
ACAATGGACTATGCCTTGGTTTTTCACTAATCAAAATCAAAATTACTCYTTAAC
ATGATAAATGAATTTACCAGTTTAGTATGCTGTGGTATTTTAATAAGTTTTCAA
AGATAATTGGGAAAACATGAGACTGGTCATATTGATGAATATTGTAACATGTG
AATTGTGATCCATTTCTGATATGTCTTGAACTACTGTGTCTAGTGGGCAAATGTC
ATTGTTACCTCTGTGTGTTAAGAAAATAATATTTTCTAAAGGTCTGT
SEQ ID NO: 20 = Human AGL isoform 1-transcript variant 4
(GenBank Accession Number-NM_000028)
CTGTCTACGGCAGCTATTCCAGAGGCAACAACTGCTTCCCTCTGTTCTCATCTCC
CCATTGGTGGCTGGCGACCCGAATTTGGGAATGGGGAGATTGCCCACCTGTTAT
CTTTGAGCAGACTAATCTCTTGTAAGCAGAAGTGCCATTCGGAGTCTCCAGAGC
CCTGTGGCTTGGGGCTGGGAATGTCCCCCTGACTTCAGGCTTTCCTAAGTGTAT
TGCTTTTCTCTGAGAATGGTCTAGGTTTTTAATTTTTTAATTGTAAGAATCTGTA
ATACAGCATTTTTATTTCGGTCTTATTCGTTGTGCTCAAAGGCAGGAAACAACT
ATTAATTTGCCTTCTCGAATCTTAATAGTTATAAGATTCATTCTCTTTCATTGCT
CTGCTAGGCATAAAACACACTTCGAACATGGGTAACTCATTCGACTGTGGAGTT
CTTTTAATTCTTATGAAAGATTTCAAATCCTCTAGAAGCCAAAATGGGACACAG
TAAACAGATTCGAATTTTACTTCTGAACGAAATGGAGAAACTGGAAAAGACCC
TCTTCAGACTTGAACAAGGGTATGAGCTACAGTTCCGATTAGGCCCAACTTTAC
AGGGAAAAGCAGTTACCGTGTATACAAATTACCCATTTCCTGGAGAAACATTTA
ATAGAGAAAAATTCCGTTCTCTGGATTGGGAAAATCCAACAGAAAGAGAAGAT
GATTCTGATAAATACTGTAAACTTAATCTGCAACAATCTGGTTCATTTCAGTATT
ATTTCCTTCAAGGAAATGAGAAAAGTGGTGGAGGTTACATAGTTGTGGACCCC
ATTTTACGTGTTGGTGCTGATAATCATGTGCTACCCTTGGACTGTGTTACTCTTC
AGACATTTTTAGCTAAGTGTTTGGGACCTTTTGATGAATGGGAAAGCAGACTTA
GGGTTGCAAAAGAATCAGGCTACAACATGATTCATTTTACCCCATTGCAGACTC
TTGGACTATCTAGGTCATGCTACTCCCTTGCCAATCAGTTAGAATTAAATCCTG
ACTTTTCAAGACCTAATAGAAAGTATACCTGGAATGATGTTGGACAGCTAGTGG
AAAAATTAAAAAAGGAATGGAATGTTATTTGTATTACTGATGTTGTCTACAATC
ATACTGCTGCTAATAGTAAATGGATCCAGGAACATCCAGAATGTGCCTATAATC
TTGTGAATTCTCCACACTTAAAACCTGCCTGGGTCTTAGACAGAGCACTTTGGC
GTTTCTCCTGTGATCTTTGCAGAAGGGAAATACAAAGAAAAGGGAATACCTGCT
TTGATTGAAAATGATCACCATATGAATTCCATCCGAAAAATAATTTGGGAGGAT
ATTTTTCCAAAGCTTAAACTCTGGGAATTTTTCCAAGTAGATGTCAACAAAGCG
GTTGAGCAATTTAGAAGACTTCTTACACAAGAAAATAGGCGAGTAACCAAGTC
TGATCCAAACCAACACCTTACGATTATTCAAGATC7CTGAATACAGACGGTTTGG
CTGTACTGTAGATATGAACATTGCACTAACGACTTTCATACCACATGACAAGGG
GCCAGCAGCAATTGAAGAATGCTGTAATTGGTTTCATAAAAGAATGGAGGAAT
TAAATTCAGAGAAGCATCGACTCATTAACTATCATCAGGAACAGGCAGTTAATT
GCCTTTTGGGAAATGTGTTTTATGAACGACTGGCTGGCCATGGTCCAAAACTAG
GACCTGTCACTAGAAAGCATCCTTTAGTTACCAGGTATTTTACTTTCCCATTTGA
AGAGATAGACTTCTCCATGGAAGAATCTATGATTCATCTGCCAAATAAAGCTTG
TTTTCTGATGGCACACAATGGATGGGTAATGGGAGATGATCCTCTTCGAAACTT
TGCTGAACCGGGTTCAGAAGTTTACCTAAGGAGAGAACTTATTTGCTGGGGAG
ACAGTGTTAAATTACGCTATGGGAATAAACCAGAGGACTGTCCTTATCTCTGGG
CACACATGAAAAAATACACTGAAATCTGCAACTTATTTCCAGGGAGTACGT
CTTGATAACTGCCACTCAACACCTCTTCACGTAGCTGAGTACATGTTGGATGCT
GCTAGGAATTTGCAACCCAATTTATATGTAGTAGCTGAACTGTTCACAGGAAGT
GAAGATCTGGACAATGTCTTTGTTACTAGACTGGGCATTAGTTCCTTAATAAGA
GAGGCAATGAGTGCATATAATAGTCATGAAGAGGGCAGATTAGTTTACCGATA
TGGAGGAGAACCTGTTGGATCCTTTGTTCAGCCCTGTTTGAGGCCTTTAATGCC
AGCTATTGCACATGCCCTGTTTATGGATATTACGCATGATAATGAGTGTCCTAT
TGTGCATAGATCAGCGTATGATGCTCTTCCAAGTACTACAATTGTTTCTATGGC
ATGTTGTGCTAGTGGAAGTACAAGAGGCTATGATGAATTAGTGCCTCATCAGAT
TTCAGTGGTTTCTGAAGAACGGTTTTACACTAAGTGGAATCCTGAAGCATTGCC
TTCAAACACAGGTGAAGTTAATTTCCAAAGCGGCATTATTGCAGCCAGGTGTGC
TATCAGTAAACTTCATCAGGAGCTTGGAGCCAAGGGTTTTATTCAGGTGTATGT
GGATCAAGTTGATGAAGACATAGTGGCAGTAACAAGACACTCACCTAGCATCC
ATCAGTCTGTTGTGGCTGTATCTAGAACTGCTTTCAGGAATCCCAAGACTTCAT
TTTACAGCAAGGAAGTGCCTCAAATGTGCATCCCTGGCAAAATTGAAGAAGTA
GTTCTTGAAGCTAGAACTATTGAGAGAAACACGAAACCTTATAGGAAGGATGA
GAATTCAATCAATGGAACACCAGATATCACAGTAGAAATTAGAGAACATATTC
AGCTTAATGAAAGTAAAATTGTTAAACAAGCTGGAGTTGCCACAAAAGGGCCC
AATGAATATATTCAAGANATAGAATTTGAAAACTTGTCTCCAGGAAGTGTTATT
ATATTCAGAGTTAGTCTTGATCCACATGCACAAGTCGCTGTTGGAATTCTTCGA
AATCATCTGACACAATTCAGTCCTCACTTTAAATCTGGCAGCCTAGCTGTTGAC
AATGCAGATCCTATATTAAAAATTCCTTTTGCTTCTCTTGCCTCCAGATTAACTT
TGGCTGAGCTAAATCAGATCCTTTACCGATGTGAATCAGAAGAAAAGGAAGAT
GGTGGAGGGTGCTATGACATACCAAACTGGTCAGCCCTTAAATATGCAGGTCTT
CAAGGTTTAATGTCTGTATTGGCAGAAATAAGACCAAAGAATGACTTGGGGCA
TCCTTTTTGTAATAATTTGAGATCTGGAGATTGGATGATTGACTATGTCAGTAA
CCGGCTTATTTCACGATCAGGAACTATTGCTGAAGTTGGTAAATGGTTGCAGGC
TATGTTCTTCTACCTGAAGCAGATCCCACGTTACCTTATCCCATGTTACTTTGAT
GCTATATTAATTGGTGCATATACCACTCTTCMGATACAGCATGGAAGCAGATG
TCAAGCTTTGTTCAGAATGGTTCAACCTTTGTGAAACACCTTTCATTGGGTTCAG
TTCAACTGTGTGGAGTAGGAAAATTCCCTTCCCTGCCAATTCTTTCACCTGCCCT
AATGGATGTACCTTATAGGTTAAATGAGATCACAAAAGAAAAGGAGCAATGTT
GTGTTTCTCTAGCTGCAGGCTTACCTCATTTTTCTTCTGGTATTTTCCGCTGCTGG
GGAAGGGATACTTTTATTGCACTTAGAGGTATACTGCTGATTACTGGACGCTAT
GTAGAAGCCAGGAATATTATTTTAGCATTTGCGGGTACCCTGAGGCATGGTCTC
ATTCCTAATCTACTGGGTGAAGGAATTTATGCCAGATACAATTGTCGGGATGCT
GTGTGGTGGTGGCTGCAGTGTATCCAGGATTACTGTAAAATGGTTCCAAATGGT
CTAGACATTCTCAAGTGCCCAGTTTCCAGAATGTATCCTACAGATGATTCTGCT
CCTTTGCCTGCTGGCACACTGGATCAGCCATTGTTTGAAGTCATACAGGAAGCA
ATGCAAAAACACATGCAGGGCATACAGTTCCGAGAAAGGAATGCTGGTCCCCA
GATAGATCGAAACATGAAGGACGAAGGTTTTAATATAACTGCAGGAGTTGATG
AAGAAACAGGATTTGTTTATGGAGGAAATCGTTTCAATTGTGGCACATGGATG
GATAAAATGGGAGAAAGTGACAGAGCTAGAAACAGAGGAATCCCAGCCACAC
CAAGAGATGGGTCTGCTGTGGAAATTGTGGGCCTGAGTAAATCTGCTGTTCGCT
GGTTGCTGGAATTATCCAAAAAAAATATTTTCCCTTATCATGAAGTCACAGTAA
AAAGAGATGGAAAGGCTATAAAGGTCTCATATGATGAGTGGAACAGAAAAATA
CAAGACAACTTTGAAAAGCTATTTCATGTTTCCGAAGACCCTTCAGATTTAAAT
GAAAAGCATCCAAATCTGGTTCACAAACGTGGCATATACAAAGATAGTTATGG
AGCTTCAAGTCCTTGGTGTGACTATCAGCTCAGGCCTAATTTTACCATAGCAAT
GGTTGTGGCCCCTGAGCTCTTTACTACAGAAAAAGCATGGAAAGCTTTGGAGAT
TGCAGAAAAAAAATTGCTTGGTCCCCTTGGCATGAAAACTTTAGATCCAGATGA
TATGGTTTACTGTGGAATTTATGACAATGCATTAGACAATGACAACTACAATCT
TGCTAAAGGTTTCAATTATCACCAAGGACCTGAGTGGCTGTGGCCTATTGGGTA
TTFTCTTCGTGCAAAATTATATTTTTCCAGATTGATGGGCCCGGAGACTACTGCA
AAGACTATAGTTTTGGTTAAAAATGTTCTTTCCCGACATTATGTTCATCTTGAGA
GATCCCCTTGGAAAGGACTTCCAGAACTGACCAATGAGAATGCCCAGTACTGT
CCTTTCAGCTGTGAAACACAAGCCTGGTCAATTGCTACTATTCTTGAGACACTT
TATGATTTATAGTTTATTACAGATATTAAGTATGCAATTACTTGTATTATAGGAT
GCAAGGTCATCATATGTAAATGCCTTATATGCACAGGCTCAAGTTGTTTTAAAA
ATCTCATTTATTATANTATTGATGCTCAATTAGGTAAGATTGTAAAAGCATTGA
TTTTTTTTAATGTACAGAGGTAGATTTCAATTTGAATCAGAAAGAAATATCATT
ACCAATGAAATGTGTTTGAGTTCAGTAAGAATTATTCAAATGCCTAGAAATCCA
TAGTTTGGAAAAGAAAAATCATGTCATCTTCTATTTGTACAGAAATGAAAATAA
AATATGAAAATAATGAAAGAAATGAAAAGATAGCTTTTAATTGTGGTATATAT
AATCTTCAGTAACAATACATACTGAATACGCTGTGGTTCATTAATATTAACACC
ACGTACTATAGTATTCTTAATACAGTGCTCACTGCATTTAATAAATATTTAATA
AATGATGAATGATAGAAGTTTCCATCTACAATATATGTTCCTAAATGGAGCACA
GATGTTCAAACTATGCTTTCATTTTTTCACTGATATATTAATTTTTGTGTAATGA
ATGCCAACAGTATATTTTATATGATTTACTTATGTGAGGAAACATGCAAAGCAT
TAGGAAATTTATTTCCTAAAAACAGTTTTGTAAAATTAGTATTGAGTTCTATTG
AGTATTATAAGATAGCTTACATTTTCAAAATGGAAATTGTCGGTCATATTTCTA
GAACTTTAAAGAAAAAAGAATGTTATATTAGTTTTCTAAAACTCAACTATCTTT
AGTCATGTTCAAAAATCTATTGCTAGATCATAGTAGATACTGGTTTTCTATTAA
CTCAAAACCTACATTGACAAGTTTAACATTGAGAAGAATCTTAACAAAAATAT
GGATATGAATTCAGTAGATATCTTAAATTCAATAAAATCACTGGAAGTTTTTCA
TGATAACTTATTTTAAGATGCCTTAAAAATCTTAAAGTCACAAAAGGAAAAAG
GTTTTTAACATTTACATGAGTTAACATTTTTTCATAGAACTTATTTCCTAGATAG
AATTTTTTACTGTTTTTTACTGTTTTCTTAAGAAAACAGTTAAATCATTATGCAT
TCAGTTGGAAGAAAGTAGTGGCAAGAATTCTTTCATTGCTATATAATATTCAGT
GGCTCATTTATACCTAATAAAATAATGGTATTTTAAAATAATGCTACTTTCAAA
GTAGCATTTTTTTAGTTAGTTTACAGGTTACATACCCAAAACCTTAACTATGACT
AAGAAATTAAAGAAGAAAACCAGCAAACTAAAACTTCTGGGCAGCAAAAATA
TATAAATGCTTCAGATGTCAAATACCCATGCTTGAAAGCTCGTGTAATTTACTT
TAAGATTATCTGCCTGCTCTTCTTCAAAGCTGACCTTGCTTTAGAAATAGTTTTA
ACTAGCTTAGTTTTCTGGTTTCCAAAACTAAAATAGATTAAATCCTACAAATTT
AAGGACAGTTGTGACAGTAATCTGACCACTATCTATAAATACATTGGACATTGG
TTTCCAAATCTCCCTTTCTTCTTCAGTTCCTTCCTTGTTCAATATATACCCTTCTC
TAAACTGTGCGGGTAAAAGGAATGACTGTCCTTGAGAGAACCATTAGTTTATCA
AAGGTTTATGTAGTTTTGTTGCTGTACCCTAACTTTGATATTCAGGGAGGTAGG
AAAGGTAACAGAAAACCAGCATATTTAATCAAAGCAAGAAGTAATCGCTGACA
GTTAAATGTGACCAAAAAAATTAAAAGTTCACAATTTTTTTAATGTAGCCATTT
GGGGTTATCTCTAGTAAGGCAGATACCCACGTTGGTAAATTTTTAGGATATTGT
GTTGCACTAGAAAACFAAGTGGTTCATATTTCTAATGAGGAAGATTAATGAAA
GAACATTGTTATATTCTGCGTGGTATATTTTAAAGTTTAAGAAGGCATGTTAAA
CATTATTTCCTCTATGGTAGTTAAAATACAGAATTAGATTTYTAACAGGTGTCAT
TTGACTAAACCATTCCTGTAGAATGCTTCATACTTGAGTGATGCTGGATAAGGTA
TTGTATTTCAACAATGGACTATGCCTTGGTTTTTCACTAATCAAAATCAAAATTA
CTCTTTAACATGATAAATGAATTTACCAGTTTAGTATGCTGTGGTATTTTAATAA
GTTTTCAAAGATAATTGGGAAAAACATGAGACTGGTCATATTGATGAATATTGTA
ACATGTGAATTGTGATCCATTTCTGATATGTCTTGAACTACTGTGTCTAGTGGGC
AAATGTCATTGTTACCTCTGTGTGTTAAGAAAATAAAAATATTTTCTAAAGGTC
TGT
SEQ ID NO: 21 = Human AGL isoform 2-transcript variant 5
(GenBank Accession Number-NM_000645)
TGTATAAGAATTTGCACATCCCAAGTTCTCTATGTGAATAGGAATGCGTTTCCAG
GGGAAGGAGAAAGAGACATTACAGAGCAGACAGCTCTATGATGTTTACTATAC
TTGCTAAAATGTGAAATTCAGCTAAATTGGAATACAAAGTAGTGCCAAAACAG
CATTAGGTTTGCGGAGTTATTTTAAACATAATTGAAAAATCAAGGTTTTTTAAT
ACTTTAAATAAAACATCTGTTTTTCAATGTGGTAATTTAAGTCCTACGATGAGTT
TATTAACATGTGCTTTTTATTTAGGGTATGAGCTACAGTTCCGATTAGGCCCAA
CTTTACAGGGAAAAGCAGTTACCGTGTATACAAATTACCCATTTCCTGGAGAAA
CATTTAATAGAGAAAAATTCCGTTCTCTGGATTGGGAAAATCCAACAGAAAGA
GAAGATGATTCTGATAAATACTGTAAACTTAATCTGCAACAATCTGGTTCATTT
CAGTATTATTTCCTTCAAGGAAATGAGAAAAGTGGTGGAGGTTACATAGTTGTG
GACCCCATTTTACGTGTTGGTGCTGATAATCATGTGCTACCCTTGGACTGTGTTA
CTCTTCAGACATTTTTAGCTAAGTGTTTGGGACCTTTTGATGAATGGGAAAGCA
GACTTAGGGTTGCAAAAGAATCAGGCTACAACATGATTCATTTTACCCCATTGC
AGACTCTTGGACTATCTAGGTCATGCTACTCCCTTGCCAATCAGTTAGAATTAA
ATCCTGACTTTTCAAGACCTAATAGAAAGTATACCTGGAATGATGTTGGACAGC
TAGTGGAAAAATTAAAAAAGGAATGGAATCTTTATTTGTATTACTGATGTTGTCT
ACAATCATACTGCTGCTAATAGTAAATGGATCCAGGAACATCCAGAATGTGCCT
ATAATCTTGTGAATTCTCCACACTTAAAACCTGCCTGGGTCTTAGACAGAGCAC
TTTGGCGTTTCTCCTGTGATGTTGCAGAAGGGAAATACAAAGAAAAGGGAATA
CCTGCTTTGATTGAAAATGATCACCATATGAATTCCATCCGAAAAATAATTTGG
GAGGATATTTTTCCAAAGCTTAAACTCTGGGAATTTTTCCAAGTAGATGTCAAC
AAAGCGGTTGAGCAATTTAGAAGACTTCTTACACAAGAAAATAGGCGAGTAAC
CAAGTCTGATCCAAACCAACACCTTACGATTATTGAGATCCTGAATACAGACG
GTTTGGCTGTACTGTAGATATGAACATTGCACTAACGACTTTCATACCACATGA
CAAGGGGCCAGCAGCAATTGAAGAATGCTGTAATTGGTTTCATAAAAGAATGG
AGGAATTAAATTCAGAGAAGCATCGACTCATTAACTATCATCAGGAACAGGCA
GTTAATTGCCTTTTGGGAAATGTGTTTTATGAACGACTGGCTGGCCATGGTCCA
AAACTAGGACCTGTCACTAGAAAGCATCCTTTAGTTACCAGGTATTTTACTTTC
CCATTTGAAGAGATAGACTTCTCCATGGAAGAATCTATGATTCATCTGCCAAAT
AAAGCTTGTTTTCTGATGGCACACAATGGATGGGTAATGGGAGATGATCCTCTT
CGAAACTTTGCTGAACCGGGTTCAGAAGTTTACCTAAGGAGAGAACTTATTTGC
TGGGGAGACAGTGTTAAATTACGCTATGGGAATAAACCAGAGGACTGTCCTTA
TCTCTGGGCACACATGAAAAAATACACTGAAATAACTGCAACTTATTTCCAGGG
AGTACGTCTTGATAACTGCCACTCAACACCTCTTCACGTAGCTGAGTACATGTT
GGATGCTGCTAGGAATTTGCAACCCAATTTATATGTAGTAGCTGAACTGTTCAC
AGGAAGTGAAGATCTGGACAATGTCTTTGTTACTAGACTGGGCATTAGTTCCTT
AATAAGAGAGGCAATGAGTGCATATAATAGTCATGAAGAGGGCAGATTAGTTT
ACCGATATGGAGGAGAACCTGTTGGATCCTTTGTTCAGCCCTGTTTGAGGCCTT
TAATGCCAGCTATTGCACATGCCCTGTTTATGGATATTACGCATGATAATGAGT
GTCCTATTGTGCATAGATCAGCGTATGATGCTCTTCCAAGTACTACAATTGTTTC
TATGGCATGTTGTGCTAGTGGAAGTACAAGAGGCTATGATGAATTAGTGCCTCA
TCAGATTTCAGTGGTTTCTGAAGAACGGTTTTACACTAAGTGGAATCCTGAAGC
ATTGCCTTCAAACACAGGTGAAGYTAATTTCCAAAGCGGCATTATTGCAGCCAG
GTGTGCTATCAGTAAACTTCATCAGGAGCTTGGAGCCAAGGGTTTTATTCAGGT
GTATGTGGATCAAGTTGATGAAGACATAGTGGCAGTAACAAGACACTCACCTA
GCATCCATCAGTCTGTTGTGGCTGTATCTAGAACTGCTTTCAGGAATCCCAAGA
CTTCATTTTACAGCAAGGAAGTGCCTCAAATGTGCATCCCTGGCAAAATTGAAG
AAGTAGTTCTTGAAGCTAGAACTATTGAGAGAAACACGAAACCTTATAGGAAG
GATGAGAATTCAATCAATGGAACACCAGATATCACAGTAGAAATTAGAGAACA
TATTCAGCTTAATGAAAGTAAAATTGTTAAACAAGCTGGAGYTGCCACAAAAG
GGCCCAATGAATATATTCAAGAAATAGAATTTGAAAACTTGTCTCCAGGAAGT
GTTATTATATTCAGAGTTAGTCTTGATCCACATGCACAAGTCGCTGTTGGAATT
CTTCGAAATCATCTGACACAATTCAGTCCTCACTTTAAATCTGGCAGCCTAGCT
GTTGACAATGCAGATCCTATATTAAAAATTCCTTTTGCTTCTCTTGCCTCCAGAT
TAACTTTGGCTGAGCTAAATCAGATCCTTTACCGATGTGAATCAGAAGAAAAG
GAAGATGGTGGAGGGTGCTATGACATACCAAACTGGTCAGCCCTTAAATATGC
AGGTCTTCAAGGTTTAATGTCTGTATTGGCAGAAATAAGACCAAAGAATGACTT
GGGGCATCCTTTTTGTAATAATTTGAGATCTGGAGATTGGATGATTGACTATGT
CAGTAACCGGCTTATTTCACGATCAGGAACTATTGCTGAAGTTGGTAAATGGTT
GCAGGCTATGTTCTTCTACCTGAAGCAGATCCCACGTTACCTTATCCCATGTTAC
TTTGATGCTATATTAATTGGTGCATATACCACTCTTCTGGATACAGCATGGAAG
CAGATGTCAAGCTTTGTTCAGAATGGTTCAACCTTTGTGAAACACCTTTCATTG
GGTTCAGTTCAACTGTGTGGAGTAGGAAAATTCCCTTCCCTGCCAATTCTTTCA
CCTGCCCTAATGGATGTACCTTATAGGTTAAATGAGATCACAAAAGAAAAGGA
GCAATGTTGTGTTTCTCTAGCTGCAGGCTTACCTCATTTTTCTTCTGGTATTTTCC
GCTGCTGGGGAAGGGATACTTTTATTGCACTTAGAGGTATACTGCTGATTACTG
GACGCTATGTAGAAGCCAGGAATATTATTTTAGCATTTGCGGGTACCCTGAGGC
ATGGTCTCATTCCTAATCTACTGGGTGAAGGAATTTATGCCAGATACAATTGTC
GGGATGCTGTGTGGTGGTGGCTGCAGTGTATCCAGGATTACTGTAAAATGGTTC
CAAATGGTCTAGACATTCTCAAGTGCCCAGTTTCCAGAATGTATCCTACAGATG
ATTCTGCTCCTTTGCCTGCTGGCACACTGGATCAGCCATTGTTTGAAGTCATACA
GGAAGCAATGCAAAAACACATGCAGGGCATACAGTTCCGAGAAAGGAATGCT
GGTCCCCAGATAGATCGAAACATGAAGGACGAAGGTTTTAATATAACTGCAGG
AGTTGATGAAGAAACAGGATTTGTTTATGGAGGAAATCGTTTCAATTGTGGCAC
ATGGATGGATAAAATGGGAGAAAGTGACAGAGCTAGAAACAGAGGAATCCCA
GCCACACCAAGAGATGGGTCTGCTGTGGAAATTGTGGGCCTGAGTAAATCTGC
TGTTCGCTGGTTGCTGGAATTATCCAAAAAAAATATTTTCCCTTATCATGAAGT
CACAGTAAAAAGACATGGAAAGGCTATAAAGGTCTCATATGATGAGTGGAACA
GAAAAATACAAGACAACTTTGAAAAGCTATTTCATGTTTCCGAAGACCCTTCAG
ATTTAAATGAAAAGCATCCAAATCTGGTTCACAAACGTGGCATATACAGAT
AGTTATGGAGCTTCAAGTCCTTGGTGTGACTATCAGCTCAGGCCTAATTTTACC
ATAGCAATGGTTGTGGCCCCTGAGCTCTTTACTACAGAAAAAGCATGGAAAGC
TTTGGAGATTGCAGAAAAAAAATTGCTTGGTCCCCTTGGCATGAAAACTTTAGA
TCCAGATGATATGGTTTACTGTGGAATTTATGACAATGCATTAGACAATGACAA
CTACAATCTTGCTAAAGGTTTCAATTATCACCAAGGACCTGAGTGGCTGTGGCC
TATTGGGTATTTTCTTCGTGCAAAATTATATTTTTCCAGATTGATGGGCCCGGAG
ACTACTGCAAAGACTATAGTTTTGGTTAAAAATGTTCTTTCCCGACATTATGTTC
ATCTTGAGAGATCCCCTTGGAAAGGACTTCCAGAACTGACCAATGAGAATGCC
CAGTACTGTCCTTTCAGCTGTGAAACACAAGCCTGGTCAATTGCTACTATTCTT
GAGACACTTTATGATTTATAGTTTATTACAGATATTAAGTATGCAATTACTTGTA
TTATAGGATGCAAGGTCATCATATGTAAATGCCTTATATGCACAGGCTCAAGTT
GTTTTAAAAATCTCATTTATTATAATATTGATGCTCAATTAGGTAAGATTGTAA
AAGCATTGATTTTTTTTAATGTACAGAGGTAGATTTCAATTTGAATCAGAAAGA
AATATCATTACCAATGAAATGTGTTTGAGTTCAGTAAGAATTATTCAAATGCCT
AGAAATCCATAGTTTGGAAAAGAAAAATCATGTCATCTTCTATTTGTACAGAAA
TGAAAATAAAATATGAAAATAATGAAAGAAATGAAAAGATAGCTTTTAATTGT
GGTATATATAATCTTCAGTAACAATACATACTGAATACGCTGTGGTTCATTAAT
ATTAACACCACGTACTATAGTATTCTTAATACAGTGCTCACTGCATTTAATAAA
TATTTAATAAATGATGAATGATAGAAGTTTCCATCTACAATATATGTTCCTAAA
TGGAGCACAGATGTTCAAACTATGCTTTCATTTTTTCACTGATATATTAATTTTT
GTGTAATGAATGCCAACAGTATATTTTATATGATTTACTTATGTGAGGAAACAT
GCAAAGCATTAGGAAATTTATTTCCTAAAAACAGTTTTGTAAAATTAGTATTGA
GTTCTATTGAGTATTATGATAGCTTACATTTTCAAAATGGAAATTGTCGGTC
ATATTTCTAGAACTTTAAAGAAAAAAGAATGTTATATTAGTTTTCTAAAACTCA
ACTATCTTTAGTCATGTTCAAAAATCTATTGCTAGATCATAGTAGATACTGGTTT
TCTATTAACTCAAAACCTACATTGACAAGTTTAACATTGAGAAGAATCTTAACA
AAAATATGGATATGAATTCAGTAGATATCTTAAATTCAATAAAATCACTGGAA
GTTTTTCATGATAACTTATTTTAAGATGCCTTAAAAATCTTAAAGTCACAAAAG
GAAAAAGGTTTTTAACATTTACATGAGTTAACATTTTTTCATAGAACTTATTTCC
TAGATAGAATTTTTTACTGTTTTTTACTGTTTTCTTAAGAAAACAGTTAAATCAT
TATGCATTCAGTTGGAAGAAAGTAGTGGCAAGAATTCTTTCATTGCTATATAAT
ATTCAGTGGCTCATTTATACCTAATAAAATAATGGTATTTTAAAATAATGCTAC
TTTCAAAGTAGCATTTTTTTAGTTAGTTTACAGGTTACATACCCAAAACCTTAAC
TATGACTAAGAAATTAAAGAAGAAAACCAGCAAACTAAAACTTCTGGGCAGCA
AAAATATATAAATGCTTCAGATGTCAAATACCCATGCTTGAAAGCTCGTGTAAT
TTACTTTAAGATTATCTGCCTGCTCTTCTTCAAAGCTGACCTTGCTTTAGAAATA
GTTTTAACTAGCTTAGTTTTCTGGTTTCCAAAACTAAAATAGATTAAATCCTACA
AATTTAAGGACAGTTGTGACAGTAATCTGACCACTATCTATAAATACATTGGAC
ATTGGTTTCCAAATCTCCCTTTCTTCTTCAGTTCCTTCCTTGTTCAATATATACCC
TTCTCTAAACTGTGCGGGTAAAAGGAATGACTGTCCTTGAGAGAACCATTAGTT
TATCAAAGGTTTATGTAGTTTTGTTGCTGTACCCTAACTTTGATATTCAGGGAGG
TAGGAAAGGTAACAGAAAACCAGCATATTTAATCAAAGCAAGAAGTAATCGCT
GACAGTTAAATGTGACCAAAAAAATTAAAAGTTCACAATTTTTTTAATGTAGCC
ATTTGGGGTTATCTCTAGTAAGGCAGATACCCACGTTGGTAAATTTTTAGGATA
TTGTGTTGCACTAGAAAACTAAGTGGTTCATATTTCTAATGAGGAAGATTAATG
AAAGAACATTGTTATATTCTGCGTGGTATATTTTAAAGTTTAAGAAGGCATGTT
AAACATTATTTCCTCTATGGTAGTTAAAATACAGAATTAGATTTTTAACAGGTG
TCATTTGACTAAACGTTTCGGTAGAATGCTTCATACTTGAGTGATGCTGGATAA
GGTATTGTATTTCAACAATGGACTATGCCTTGGTTTTTCACTAATCAAAATCAA
AATTACTCTTTAACATGATAAATGAATTTACCAGTTTAGTATGCTGTGGTATTTT
AATAAGTTTTCAAAGATAATTGGGAAAACATGAGACTGGTCATATTGATGAAT
ATTGTAACATGTGAATTGTGATCCATTTCTGATATGTCTTGAACTACTGTGTCTA
GTGGGCAAATGTCATTGTTACCTCTGTGTGTTAAGAAAATAAAAATATTTTCTA
AAGGTCTGT
SEQ ID NO: 22 = Human AGL isoform 3-transcript variant 6
(GenBank Accession Number-NM_000646)
GGGTAACTCATTCGACTGTGGAGTTCTTTTAATTCTTATGAAAGATTTCAAATCC
TCTAGAAGCCAAAATGGGACACAGTAAACAGATTCGAATTTTACTTCTGAACG
AAATGGAGAAACTGGAAAAGACCCTCTTCAGACTTGAACAAGAAACTGGGTCT
CACTATGTTGCCCAGGTTGATATTGAACTCCTGGACTCAAGCAACCCTCCCTCT
TTGGCCTCTGAAAGTACTGGGATTACAAGCATAAGCCACCGGGCATGGCCCCA
ATTCTGAGCATTAATTTATTTATTGGGTATGAGCTACAGTTCCGATTAGGCCCA
ACTTTACAGGGAAAAGCAGTTACCGTGTATACAAATTACCCATTTCCTGGAGAA
ACATTTAATAGAGAAAAATTCCGTTCTCTGGATTGGGAAAATCCAACAGAAAG
AGAAGATGATTCTGATAAATACTGTAAACTTAATCTGCAACAATCTGGTTCATT
TCAGTATTATTTCCTTCAAGGAAATGAGAAAAGTGGTGGAGGTTACATAGTTGT
GGACCCCATTTTACGTGTTGGTGCTGATAATCATGTGCTACCCTTGGACTGTGTT
ACTCTTCAGACATTTTTAGCTAAGTGTTTGGGACCTTTTGATGAATGGGAAAGC
AGACTTAGGGTTGCAAAAGAATCAGGCTACAACATGATTCATTTTACCCCATTG
CAGACTCTTGGACTATCTAGGTCATGCTACTCCCTTGCCAATCAGTTAGAATTA
AATCCTGACTTTTCAAGACCTAATAGAAAGTATACCTGGAATGATGTTGGACAG
CTAGTGGAAAAATTAAAAAAGGAATGGAATGTTATTTGTATTACTGATGTTGTC
TACAATCATACTGCTGCTAATAGTAAATGGATCCAGGAACATCCAGAATGTGCC
TATAATCTTGTGAATTCTCCACACTTAAAACCTGCCTGGGTCTTAGACAGAGCA
CTTTGGCGTTTCTCCTGTGATGTTGCAGAAGGGAAATACAAAGAAAAGGGAAT
ACCTGCTTTGATTGAAAATGATCACCATATGAATTCCATCCGAAAAATAATTTG
GGAGGATATTTTTCCAAAGCTTAAACTCTGGGAATTTTTCCAAGTAGATGTCAA
CAAAGCGGTTGAGCAATTTAGAAGACTTCTTACACAAGAAAATAGGCGAGTAA
CCAAGTCTGATCCAAACCAACACCTTACGATTATTCAAGATCCTGANTACAGAC
GGTTTGGCTGTACTGTAGATATGAACATTGCACTAACGACTTTCATACCACATG
ACAAGGGGCCAGCAGCAATTGAAGAATGCTGTAATTGGTTTCATAAAAGAATG
GAGGAATTAAATTCAGAGAAGCATCGACTCATTAACTATCATCAGGAACAGGC
AGTTAATTGCCTTTTGGGAAATGTGTTTTATGAACGACTGGCTGGCCATGGTCC
AAAACTAGGACCTGTCACTAGAAAGCATCCTTTAGTTACCAGGTATTTTACTTT
CCCATTTGAAGAGATAGACTTCTCCATGGAAGAATCTATGATTCATCTGCCAAA
TAAAGCTTGTTTTCTGATGGCACACAATGGATGGGTAATGGGAGATGATCCTCT
TCGAAACTTTGCTGAACCGGGTTCAGAAGTTTACCTAAGGAGAGAACTTATTTG
CTGGGGAGACAGTGTTAAATTACGCTATGGGAATAAACCAGAGGACTGTCCTT
ATCTCTGGGCACACATGAAAAAATACACTGAAATAACTGCAACTTATTTCCAGG
GAGTACGTCTTGATAACTGCCACTCAACACCTCTTCACGTAGCTGAGTACATGT
TGGATGCTGCTAGGAATTTGCAACCCAATTTATATGTAGTAGCTGAACTGTTCA
CAGGAAGTGAAGATCTGGACAATGTCTTTGTTACTAGACTGGGCATTAGTTCCT
TAATAAGAGAGGCAATGAGTGCATATAATAGTCATGAAGAGGGCAGATTAGTT
TACCGATATGGAGGAGAACCTGTTGGATCCTTTGTTCAGCCCTGTTTGAGGCCT
TTAATGCCAGCTATTGCACATGCCCTGTTTATGGATATTACGCATGATAATGAG
TGTCCTATTGTGCATAGATCAGCGTATGATGCTCTTCCAAGTACTACAATTGTTT
CTATGGCATGTTGTGCTAGTGGAAGTACAAGAGGCTATGATGAAYTAGTGCCTC
ATCAGATTTCAGTGGTTTCTGAAGAACGGTTTTACACTAAGTGGAATCCTGAAG
CATTGCCTTCAAACACAGGTGAAGTTAATTTCCAAAGCGGCATTATTGCAGCCA
GGTGTGCTATCAGTAAACTTCATCAGGAGCTTGGAGCCAAGGGTTTTATTCAGG
TGTATGTGGATCAAGTTGATGAAGACATAGTGGCAGTAACAAGACACTCACCT
AGCATCCATCAGTCTGTTGTGGCTGTATCTAGAACTGCTTTCAGGAATCCCAAG
ACTTCATTTTACAGCAAGGAAGTGCCTCAAATGTGCATCCCTGGCAAAATTGAA
GAAGTAGTTCTTGAAGCTAGAACTATTGAGAGAAACACGAAACCTTATAGGAA
GGATGAGAATTCAATCAATGGAACACCAGATATCACAGTAGAAATTAGAGAAC
ATATTCAGCTTAATGAAAGTAAAATTGTTAAACAAGCTGGAGTTGCCACAAAA
GGGCCCAATGAATATATTCAAGATAGAATTTGAAAACTTGTCTCCAGGAAG
TGTTATTATATTCAGAGTTAGTCTTGATCCACATGCACAAGTCGCTGTTGGAATT
CTTCGAAATCATCTGACACAATTCAGTCCTCACTTTAAATCTGGCAGCCTAGCT
GTTGACAATGCAGATCCTATATTAAAAATTCCTTTTGCTTCTCTTGCCTCCAGAT
TAACTTTGGCTGAGCTAAATCAGATCCTTTACCGATGTGAATCAGAAGAAAAG
GAAGATGGTGGAGGGTGCTATGACATACCAAACTGGTCAGCCCTTAAATATGC
AGGTCTTCAAGGTTTAATGTCTGTATTGGCAGAAATAAGACCAAAGAATGACTT
GGGGCATCCTTTTTGTAATAATTTGAGATCTGGAGATTGGATGATTGACTATGT
CAGTAACCGGCTTATTTCACGATCAGGAACTATTGCTGAAGTTGGTAAATGGTT
GCAGGCTATGTTCTTCTACCTGAAGCAGATCCCACGTTACCTTATCCCATGTTAC
TTTGATGCTATATTAATTGGTGCATATACCACTCTTCTGGATACAGCATGGAAG
CAGATGTCAAGCTTTGTTCAGAATGGTTCAACCTTTGTGAAACACCTTTCATTG
GGTTCAGTTCAACTGTGTGGAGTAGGAAAATTCCCTTCCCTGCCAATTCTTTCA
CCTGCCCTAATGGATGTACCTTATAGGTTAAATGAGATCACAAAAGAAAAGGA
GCAATGTTGTGTTTCTCTAGCTGCAGGCTTACCTCATTTTTCTTCTGGTATTTTCC
GCTGCTGGGGAAGGGATACTTTTATTGCACTTAGAGGTATACTGCTGATTACTG
GACGCTATGTAGAAGCCAGGAATATTATTTTAGCATTTGCGGGTACCCTGAGGC
ATGGTCTCATTCCTAATCTACTGGGTGAAGGAATTTATGCCAGATACAATTGTC
GGGATGCTGTGTGGTGGTGGCTGCAGTGTATCCAGGATTACTGTAAAATGGTTC
CAAATGGTCTAGACATTCTCAAGTGCCCAGTTTCCAGAATGTATCCTACAGATG
ATTCTGCTCCTTTGCCTGCTGGCACACTGGATCAGCCATTGYTTGAAGTCATACA
GGAAGCAATGCAAAAACACATGCAGGGCATACAGTTCCGAGAAAGGAATGCT
GGTCCCCAGATAGATCGAAACATGAAGGACGAAGGTTTTAATATAACTGCAGG
AGTTGATGAAGAAACAGGATTTCTTTTATGGAGGAAATCGTTTCAATTGTGGCAC
ATGGATGGATAAAATGGGAGAAAGTGACAGAGCTAGAAACAGAGGAATCCCA
GCCACACCAAGAGATGGGTCTGCTGTGGAAATTGTGGGCCTGAGTAAATCTGC
TGTTCGCTGGTTGCTGGAATTATCCAAAAAAAATATTTTCCCTTATCATGAAGT
CACAGTAAAAAGACATGGAAAGGCTATAAAGGTCTCATATGATGAGTGGAACA
GAAAAATACAAGACAACTTTGAAAAGCTATTTCATGTTTCCGAAGACCCTTCAG
ATTTAAATGAAAAGCATCCAAATCTGGTTCACAAACGTGGCATATACAAAGAT
AGTTATGGAGCTTCAAGTCCTTGGTGTGACTATCAGCTCAGGCCTAATTTTACC
ATAGCAATGGTTGTGGCCCCTGAGCTCTTTACTACAGAAAAAGCATGGAAAGC
TTTGGAGATTGCAGAAAAAATTGCTTGGTCCCCTTGGCATGAAAACTTTAGA
TCCAGATGATATGGTTTACTGTGGAATTTATGACAATGCATTAGACAATGACAA
CTACAATCTTGCTAAAGGTTTCAATTATCACCAAGGACCTGAGTGGCTGTGGCC
TATTGGGTATTTTCTTCGTGCAAAATTATATTTTTCCAGATTGATGGGCCCGGAG
ACTACTGCAAAGACTATAGTTTTGGTTAAAAATGTTCTTTCCCGACATTATGTTC
ATCTTGAGAGATCCCCTTGGAAAGGACTTCCAGAACTGACCAATGAGAATGCC
CAGTACTGTCCTTTCAGCTGTGAAACACAAGCCTGGTCAATTGCTACTATTCTT
GAGACACTTTATGATTTATAGTTTATTACAGATATTAAGTATGCAATTACTTGTA
TTATAGGATGCAAGGTCATCATATGTAAATGCCTTATATGCACAGGCTCAAGTT
GTTTTAAAAATCTCATTTATTATAATATTGATGCTCAATTAGGTAAGATTGTAA
AAGCATTGATTTTTTTTAATGTACAGAGGTAGATTTCAATTTGAATCAGAAAGA
AATATCATTACCAATGAAATGTGTTTGAGTTCAGTAAGAATTATTCAAATGCCT
AGAAATCCATAGTTTGGAAAAGAAAAATCATGTCATCTTCTATTTGTACAGAAA
TGAAAATAAAATATGAAAATAATGAAAGAAATGAAAAGATAGCTTTTAATTGT
GGTATATATAATCTTCAGTAACAATACATACTGAATACGCTGTGGTTCATTAAT
ATTAACACCACGTACTATAGTATTCTTAATACAGTGCTCACTGCATTTAATAAA
TATTTAATAAATGATGAATGATAGAAGTTTCCATCTACAATATATGTTCCTAAA
TGGAGCACAGATGTTCAAACTATGCTTTCATTTTTTCACTGATATATTAATTTTT
GTGTAATGAATGCCAACAGTATATTTTATATGATTTACTTATGTGAGGAAACAT
GCAAAGCATTAGGAAATTTATTTCCTAAAAACAGTTTTGTAAAATTAGTATTGA
GTTCTATTGAGTATTATAAGATAGCTTACATTTTCAAAATGGAAATTGTCGGTC
ATATTTCTAGAACTTTAAAGAAAAAAGAATGTTATATTAGTTTTCTAAAACTCA
ACTATCTTTAGTCATGTTCAAAAATCTATTGCTAGATCATAGTAGATACTGGTTT
TCTATTAACTCAAAACCTACATTGACAAGTTTAACATTGAGAAGAATCTTAACA
AAAATATGGATATGAATTCAGTAGATATCTTAAATTCAATAAAATCACTGGAA
GTTTTTCATGATAACTTATTTTAAGATGCCTTAAAAATCTTAAACTCACAAAAG
GAAAAAGGTTTTTAACATTTACATGAGTTAACATTTTTTCATAGAACTTATTTCC
TAGATAGAATTTTTTACTGTTTTTTACTGTTTTCTTAAGAAAACAGTTAAATCAT
TATGCATTCAGTTGGAAGAAAGTAGTGGCAAGAATTCTTTCATTGCTATATAAT
ATTCAGTGGCTCATTTATACCTAATAAAATAATGGTATTTTAAAATAATGCTAC
TTTCAAAGTAGCATTTTTTTAGTTAGTTTACAGGTTACATACCCAAAACCTTAAC
TATGACTAAGAAATTAAAGAAGAAAACCAGCAAACTAAAACTTCTGGGCAGCA
AAAATATATAAATGCTTCAGATGTCAAATACCCATGCTTTGAAAGCTCGTGTAAT
TTACTTTAAGATTATCTGCCTGCTCTTCTTCAAAGCTGACCTTGCTTTAGAAATA
GTTTTAACTAGCTTAGTTTTCTGGTTTCCAAAACTAAAATAGATTAAATCCTACA
AATTTAAGGACAGTTGTGACAGTAATCTGACCACTATCTATAAATACATTGGAC
ATTGGTTTCCAAATCTCCCTTTCTTCTTCAGTTCCTTCCTTGTTCAATATATACCC
TTCTCTAAACTGTGCGGGTAAAAGGAATGACTGTCCTTGAGAGAACCATTAGTT
TATCAAAGGTTTATGTAGTTTTGTTGCTGTACCCTAACTTTGATATTCAGGGAGG
TAGGAAAGGTAACAGAAAACCAGCATATTTAATCAAAGCAAGAAGTAATCGCT
GACAGTTAAATGTGACCAAAAAAATTAAAAGTTCACAATTTTTTTAATGTAGCC
ATTTGGGGTTATCTCTAGTAAGGCAGATACCCACGTTGGTAAATTTTTAGGATA
TTGTGTTGCACTAGAAAACTAAGTGGTTCATATTTCTAATGAGGAAGATTAATG
AAAGAACATTGTTATATTCTGCGTGGTATATTTTAAAGTTTAAGAAGGCATGTT
AAACATTATTTCCTCTATGGTAGTTAAAATACAGAATTAGATTTTTAACAGGTG
TCATTTGACTAAACGTTTCGGTAGAATGCTTCATACTTGAGTGATGCTGGATAA
GGTATTGTATTTCAACAATGGACTATGCCTTGGTTTTTCACTAATCAAAATCAA
AATTACTCTTTAACATGATAAATaAATTTACCAGTTTAGTATGCTGTGGTATTTT
AATAAGTTTTCAAAGATAATTGGGAAAACATGAGACTGGTCATATTGATGAAT
ATTGTAACATGTGAATTGTGATCCATTTCTGATATGTCTTGAACTACTGTGTCTA
GTGGGCAAATGTCATTGTTACCTCTGTGTGTTAAGAAAATAAAAATATTTTCTA
AAGGTCTGT
SEQ ID NO: 23 = His Tag
HHHHHH
SEQ ID NO: 24 = c-myc, tag
EQKLISEEDL
SEQ ID NO: 25
AGIH
SEQ ID NO: 26
SAGIH
SEQ ID NO: 27-heavy chain variable (VH) domain CDR1 of exemplary 3E10 V_{H}
(as that VH is defined with reference to SEQ ID NO: 6), in accordance
with the IMGT system
GFTFSNYG
SEQ ID NO: 28-heavy chain variable (VH) domain CDR2 of exemplary 3E10 V_{H}
(as that VH is defined with reference to SEQ ID NO: 6), in accordance
with the IMGT system
ISSGSSTI
SEQ ID NO: 29-heavy chain variable (VH) domain CDR3 of exemplary 3E10 V_{H}
(as that VH is defined with reference to SEQ ID NO: 6), in accordance
with the IMGT system
ARRGLLLDY
SEQ ID NO: 30-light chain variable (VI) domain CDR1 of exemplary 3E10 V_{L}
(as that VL is defined with reference to SEQ ID NO: 8), in accordance
with the IMGT system
KSVSTSSYSY
SEQ ID NO: 31-light chain variable (VL) domain CDR2 of exemplary 3E10 V_{L}
(as that VL is defined with reference to SEQ ID NO: 8), in accordance
with the IMGT system
YAS
SEQ ID NO: 32-light chain variable (VL) domain CDR3 of exemplary 3E10 V_{L}
(as that VL is defined with reference to SEQ ID NO: 8), in accordance
with the IMGT system
QIISREFPWT
SEQ ID NO: 33-(G₄S)n, wherein n is an integer from 1-10
(GGGGS)ₙ
SEQ ID NO: 34-ASSLNIA horning peptide
ASSLNIA
SEQ ID NO: 35-Arg7 peptide
RRRRRRR
SEQ ID NO: 36-KFERQ
KTERQ

### INCORPORATION BY REFERENCE

All publications and patents mentioned herein are hereby incorporated by reference in their entirety as if each individual publication or patent was specifically and individually indicated to be incorporated by reference.

While specific embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

1.-171. (canceled)

172. A method of treating Forbes-Cori disease in a subject in need thereof, comprising contacting the cell with a chimeric polypeptide comprising: (i) a mature acid alpha-glucosidase (GAA) polypeptide and (ii) an internalizing moiety that promotes delivery into cells;
wherein the chimeric polypeptide has acid alpha-glucosidase activity, and wherein the chimeric polypeptide does not comprise a GAA precursor polypeptide of approximately 110 kilodaltons.

173. The method of claim 172, wherein the mature GAA polypeptide has a molecular weight of approximately 70-76 kilodaltons.

174. The method of claim 172, wherein the mature GAA polypeptide consists of an amino acid sequence selected from residues 122-782 of SEQ ID NO: 4 or residues 204-782 of SEQ ID NO: 5.

175. The method of claim 172, wherein the internalizing moiety promotes delivery of the chimeric polypeptide into cells.

176. (canceled)

177. (canceled)

178. The method of claim 172, wherein said chimeric polypeptide reduces cytoplasmic glycogen accumulation.

179. The method of claim 172, wherein the mature GAA polypeptide is glycosylated.

180. The method of claim 172, wherein the mature GAA polypeptide is not glycosylated.

181. (canceled)

182. The method of claim 172, wherein the internalizing moiety comprises an antibody or antigen binding fragment.

183. The method of claim 182, wherein said antibody is a monoclonal antibody or fragment thereof, or
wherein said antibody is monoclonal antibody 3E10, or an antigen binding fragment thereof.

184. (canceled)

185. The method of claim 172, wherein the internalizing moiety transits cellular membranes via an equilibrative nucleoside transporter, or
wherein the internalizing moiety transits cellular membranes via an equilibrative nucleoside transporter 2 (ENT2) transporter.

186.-189. (canceled)

190. The method of claim 182, wherein the antibody or antigen binding fragment comprises a heavy chain variable domain comprising an amino acid sequence at least 95% identical to SEQ ID NO: 6, or a humanized variant thereof, or
wherein the antibody or antigen binding fragment comprises a light chain variable domain comprising an amino acid sequence at least 95% identical to SEQ ID NO: 8, or a humanized variant thereof, or
wherein the antibody or antigen binding fragment comprises a heavy chain variable domain comprising the amino acid sequence of SEQ ID NO: 6 and a light chain variable domain comprising the amino acid sequence of SEQ ID NO: 8, or a humanized variant thereof.

191. (canceled)

192. (canceled)

193. The method of claim 182, wherein the antibody or antigen binding fragment comprises:
VH CDR1 having the amino acid sequence of SEQ ID NO 9;
VH CDR2 having the amino acid sequence of SEQ ID NO 10;
VH CDR3 having the amino acid sequence of SEQ ID NO 11;
VL CDR1 having the amino acid sequence of SEQ ID NO: 12;
VL CDR2 having the amino acid sequence of SEQ ID NO: 13; and
VL CDR3 having the amino acid sequence of SEQ ID NO: 14.

194.-199. (canceled)

200. A method of decreasing glycogen accumulation in cytoplasm of cells of a Forbes-Cori patient, comprising contacting muscle cells with a chimeric polypeptide, which chimeric polypeptide comprises (i) a mature acid alpha-glucosidase (GAA) polypeptide and (ii) an internalizing moiety that promotes transport into cytoplasm of cells;
wherein the chimeric polypeptide has acid alpha-glucosidase activity, and wherein the chimeric polypeptide does not comprise a GAA precursor polypeptide of approximately 110 kilodaltons.

201. A method of increasing GAA activity in the cytoplasm of a cell, comprising delivering a chimeric polypeptide, wherein said chimeric polypeptide comprises: (i) a mature acid alpha-glucosidase (GAA) polypeptide and (ii) an internalizing moiety that promotes transport into cytoplasm of cells;
wherein the chimeric polypeptide has acid alpha-glucosidase activity, and wherein the chimeric polypeptide does not comprise a GAA precursor polypeptide of approximately 110 kilodaltons.

202. The method of claim 201, wherein said cell is in a subject, wherein said subject has Forbes-Cori disease.

203. The method of claim 200, wherein said method is in vitro.

204. The method of claim 200, wherein the mature GAA polypeptide has a molecular weight of approximately 70-76 kilodaltons.

205. (canceled)

206. (canceled)

207. The method of claim 200, wherein the mature GAA polypeptide consists of an amino acid sequence selected from: residues 122-782 of SEQ ID NO: 4 or 5, residues 123-782 of SEQ ID NO: 4 or 5, or residues 204-782 of SEQ ID NO: 4 or 5.

208.-212. (canceled)

213. The method of claim 200, wherein the mature GAA polypeptide has a glycosylation pattern that differs from that of naturally occurring human GAA.

214. The method of claim 200, wherein the internalizing moiety promotes delivery of the chimeric polypeptide into cytoplasm of cells.

215.-235. (canceled)
